(19) United States
(12) Patent Application Publication
(10) Pub. No.: US 2025/0090495 A1
(43) Pub. Date: Mar. 20, 2025

(54) CDC7 Inhibitor Combinational Therapy

(71) Applicant: Lin Bioscience, Inc., Taipei (TW)

(72) Inventors: Tom LIN, Sydney (AU); Irene WANG, Taipei (TW); Mark G. Frattini, Rigewood, NJ (US); Hakim DJABALLAH, Scarsdale, NY (US); Thomas J. Kelly, New York, NY (US)

(21) Appl. No.: 18/969,747

(22) Filed: Dec. 5, 2024

Related U.S. Application Data
(63) Continuation-in-part of application No. 18/824,504, filed on Sep. 4, 2024.
Application No. 18/824,504 is a continuation of application No. 18/138,351, filed on Apr. 24, 2023, now Pat. No. 12,109,189.
Application No. 18/138,351 is a continuation of application No. 17/330,957, filed on May 26, 2021, now Pat. No. 11,666,550.
Application No. 17/330,957 is a continuation of application No. 16/189,780, filed on Nov. 13, 2018, now Pat. No. 11,045,446.
Application No. 16/189,780 is a continuation of application No. 15/728,609, filed on Oct. 10, 2017, now Pat. No. 10,123,992.
Application No. 15/728,609 is a continuation of application No. 15/349,905, filed on Nov. 11, 2016, now Pat. No. 9,782,386.
Application No. 15/349,905 is a continuation of application No. 14/936,472, filed on Nov. 9, 2015, now Pat. No. 9,492,427.
Application No. 14/936,472 is a continuation of application No. 13/583,170, filed on Oct. 18, 2012, now Pat. No. 9,180,105.
(60) Provisional application No. 61/311,741, filed on Mar. 8, 2010.

(51) Int. Cl. A61K 31/365; A61K 9/00; A61K 31/122; A61K 31/352; A61N 5/10; C07D 493/08; C07D 493/18; C09B 13/02; C09B 61/00; C12Q 1/6886

(52) U.S. Cl. CPC A61K 31/365; A61K 9/0019; A61K 31/122; A61K 31/352; A61N 5/10; C07D 493/08; C07D 493/18; C09B 13/02; C09B 61/00; C12Q 1/6886; C12Q 2600/106; C12Q 2600/156

(57) ABSTRACT

The present invention provides a method of treatment and composition for CDC7 inhibitor combinational therapy for treatment of proliferative diseases. In an embodiment, the composition of the present invention comprises a therapeutically effective amount of a combination of a CDC7 inhibitor of the present invention or a pharmaceutically acceptable salt thereof and one or more antineoplastic agents. In an embodiment, the CFC7 inhibitor comprises granaticin B. In an embodiment, the antneoplastic agent comprises azacitidine and venetoclax. The present invention further provides a method of treatment comprising the step of administration to a subject suffering from proliferative disease of any embodiment of the composition of the present invention.

### CROSS REFERENCE TO RELATED APPLICATION

The present application is a continuation in part of and claims priority under 35 U.S.C. § 120 to U.S. application Ser. No. 18/824,504 filed Sep. 4, 2024, which is a continuation of and claims priority under 35 U.S.C. § 120 to U.S. Ser. No. 18/138,351, filed Apr. 24, 2023, which is a continuation of and claims priority under 35 U.S.C. § 120 to 17/330,957 filed May 26, 2021, which is a continuation of and claims priority under 35 U.S.C. § 120 to U.S. Ser. No. 16/189,780, filed Nov. 13, 2018, which is a continuation of and claims priority under 35 U.S.C. § 120 to U.S. application, U.S. Ser. No. 15/728,609, filed Oct. 10, 2017, now U.S. Pat. No. 10,123,992, which is a continuation of and claims priority under 35 U.S.C. § 120 to U.S. application, U.S. Ser. No. 15/349,905, filed Nov. 11, 2016, now U.S. Pat. No. 9,782,386, which is a continuation of and claims priority under 35 U.S.C. § 120 to U.S. application, U.S. Ser. No. 14/936,472, filed Nov. 9, 2015, now U.S. Pat. No. 9,492,427, which is a continuation of and claims priority under 35 U.S.C. § 120 to U.S. application, U.S. Ser. No. 13/583,170, filed Oct. 18, 2012, now U.S. Pat. No. 9,180,105, which is a national stage filing under 35 U.S.C. § 371 of international PCT application, PCT/US2011/027619, filed Mar. 8, 2011, which claims priority under 35 U.S.C. § 119(e) to U.S. provisional patent application, U.S. Ser. No. 61/311,741, filed Mar. 8, 2010, each of which is incorporated herein by reference. The present application also claims priority under 35 U.S.C. § 119(e) to U.S. provisional patent application U.S. Ser. No. 63/606,581 filed Dec. 5, 2023 which is incorporated herein by reference.

### FIELD OF THE INVENTION

The present invention provides a method of treatment and composition for CDC7 inhibitor combinational therapy for treatment of proliferative diseases.

### BACKGROUND OF THE INVENTION

Understanding how the genomes of eukaryotes are duplicated during each cell cycle is a fundamental problem of modern biology and is a critical aspect of the more general problem of understanding the mechanisms that control cellular proliferation. The transition from G1 into S phase is a major decision point for the cell and is subject to elaborate controls whose mechanisms are not yet understood at the molecular level. (Bell, S. P. and A. Dutta (2002) Annu Rev Biochem 71: 333-74; Dutta, A. and S. P. Bell (1997) Annu Rev Cell Dev Biol 13: 293-332; Jallepalli, P. V. and T. J. Kelly (1997) Curr Opin Cell Biol 9 (3): 358-63; Kelly, T. J. and G. W. Brown (2000) AnnuRev Biochem 69: 829-80; Stillman, B. (1996) Science 274 (5293): 1659-64) The stability of the genome depends upon the precise operation of the DNA “replication switch,” as well as upon the proper coupling of DNA replication to other events in the cell. It has become quite clear that perturbation of any of these mechanisms can contribute to cancer. (Sherr, C. J. (1996). Science 274 (5293): 1672-7)

During the couple of decades, work in a number of laboratories has led to a dramatic advance in our understanding of cellular DNA replication (Bell, S. P. and A. Dutta, et al.; Kelly, T. J. and G. W. Brown, et al.). The analysis of simple model systems, particularly Saccharomyces cerevisiae, Schizosaccharomyces pombe, and Xenopus laevis, has resulted in the identification of proteins that act at origins of DNA replication to initiate DNA synthesis. A significant breakthrough was the discovery by Stillman and Bell of the six-subunit origin recognition complex (ORC), which binds to specific origins of DNA replication in S. cerevisiae and recruits additional initiation factors to form the pre-replication complex (pre-RC). The ORC has been conserved throughout eukaryotic evolution. (Chuang, R. Y., L. Chretien, et al. (2002) J Biol Chem 277 (19): 16920-7; Gossen, M., D. T. Pak, et al. (1995) Science 270 (5242): 1674-7; Moon, K. Y., D. Kong, et al. (1999) Proc Natl Acad Sci USA 96 (22): 12367-12372; Rowles, A., J. P. Chong, et al. (1996) Cell 87 (2): 287-96; Vashee, S., P. Simancek, et al. (2001) J Biol Chem 276 (28): 26666-73) We now know that a common set of initiation proteins assemble at replication origins in all eukaryotes and that the activities of these proteins are regulated by specific protein kinases. However, despite this progress, our understanding of the biochemical mechanisms of initiation of eukaryotic DNA replication remains quite superficial.

Genetic studies in yeasts and biochemical studies in Xenopus have demonstrated that the initiation of eukaryotic DNA replication takes place in two stages. (Bell, S. P. and A. Dutta, et al.; Kelly, T. J. and G. W. Brown, et al.) In the first stage, which lasts from late M through the G1 phase of the cell cycle, pre-RCs are assembled at origins of DNA replication. At the beginning of S phase, pre-RCs are activated by the action of two heterodimeric protein kinases, Cdc7-Dbf4 and S phase cyclin-dependent kinase (S-CDK). This event marks the transition to the second stage of initiation, during which the origin is unwound and additional proteins are recruited to form active replication forks. The presence of cyclin dependent kinase activity (and perhaps other inhibitory factors) prevents further assembly of pre-RCs during the second stage of the initiation reaction. This mechanism constitutes a “replication switch” that ensures that origins of DNA replication fire only once each cell cycle, thus preserving genomic integrity.

As noted above, the activation of the pre-RC requires the activities of Cdc7-Dbf4 and S-CDK. Both kinases are activated at the G1/S boundary when their respective regulatory subunits accumulate to sufficient levels, and both appear to associate with the pre-RC. (Brown, G. W., P. V. Jallepalli, et al. (1997) Proc. Natl. Acad. Scl, USA 94: 6142-6147; Dowell, S. J., P. Romanowski, et al. (1994) Science 265 (5176): 1243-6; Jallepalli, P. V. and T. J. Kelly; Jares, P. and J. J. Blow (2000) Genes Dev 14 (12): 528-40; Johnston, L. H., H. Masai, et al. (1999) Trends Cell Biol 9 (7): 249-52; Leatherwood, J., A. Lopez-Girona, et al. (1996) Nature 379 (6563): 360-3; Walter, J. C. (2000)/. Biol. Chem. 275 (50): 39773-8) Although the regulation of S-CDK activity has been shown to be quite complex with multiple cyclin subunits pairing with multiple Cdk subunits, Cdc7 activity is strictly regulated by the expression of the Dbf4 subunit, which is very tightly cell cycle regulated with peak expression occurring at the G1/S boundary. (Bell, S. P. and A. Dutta, et al.) The activity of Cdc7 has been shown to be required for entry into S phase of the cell cycle. Studies in yeast have shown that cells depleted of this kinase activity progress from G1 to M phase without an intervening S phase, resulting in cell death (Bell, S. P. and A. Dutta, et al.; Kelly, T. J. and G. W. Brown, et al.), and conditional knockout mouse Embryonic stem(ES) cells for Dbf4 have recently been shown to undergo S phase arrest with resultant apoptosis when gene expression is silenced. (Yamashita, N., Kim, J-M, et al. (2005) Genes to Cells 10: 551-563) Genetic evidence has shown that the six subunit Minichromosome Maintenance complex (MCM2-7), the presumed helicase activity required for origin unwinding and the initiation of DNA replication (Bell, S. P. and A. Dutta, et al.; Kelly, T. J. and G. W. Brown, et al.), is a target of regulation by the Cdc7-Dbf4 kinase, and the Mcm2 protein is an excellent substrate for the Cdc7:Dbf4 kinase in vitro. (Sclafani, R. A. (2000)/Cell Sci 113 (Pt 12): 2111-7) The MCM proteins and Cdc7 have been shown to be overexpressed in the majority of cancers including both solid tumors and hematologic malignancies. (Hess, G. F., Drong, R. F., et al. (1998) Gene 211 (1): 133-40; Velculescu, V. E., Madden, S. L., et al. (1999) Nature Genetics 23:387-88) Importantly, it has recently been shown that overexpression of Cdc7 in cutaneous melanoma samples was associated with poor risk disease and chemotherapy resistance. (Nambiar, S., Mirmohammadsadegh, A., et al. (2007) Carcinogenesis 12: 2501-2510) In addition, Cdc7 overexpression has also been shown in aggressive undifferentiated papillary thyroid carcinoma and in aggressive head and neck cancers that are positive for human papillomavirus (Fluge, O., Bruland, O., Akslen, L. A., et al. (2006) Thyroid 16 (2): 161-175; Slebos, R J. C, Yi, Y., Ely, K., et al. (2006) Clin Cancer Res 12 (3): 701-709). In fact, sensitive assay systems are being developed in Europe and the United States to detect the presence of MCM proteins in the urine of patients with genitourinary malignancies as well as breast cancer patients, and this seems to correlate with a more aggressive malignancy. Cdc7 activity is also conserved from yeast to man making it an attractive candidate for a therapeutic target. The logical interpretation of this data is that Cdc7:Dbf4 is a bona fide therapeutic target. Therefore, there is a need for an effective therapeutic regimen for treatment of neoplastic diseases. In particular, there is a need for effective combinations of CDC7 inhibitors and ant-neoplastic compounds for treatment of cancer.

### SUMMARY OF THE INVENTION

A method of treating a proliferative disorder of a subject comprising the administration of a pharmaceutical composition comprising a therapeutically effective amount of a combination of (a) a compound of Formula (A) or (B) or a pharmaceutically acceptable salt thereof and (b) one or more antineoplastic agents to the subject wherein:

A compound of Formula (A) or (B):

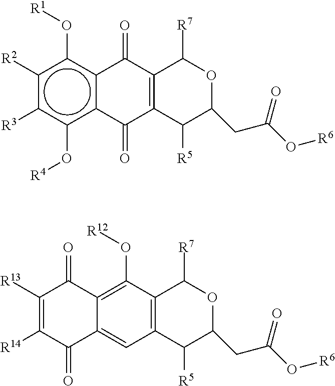

each instance of R1 and R4 is independently selected from the group consisting of hydrogen, carbonyl, silyl, optionally substituted alkyl, optionally substituted alkenyl, optionally substituted alkynyl, optionally substituted carbocyclyl, optionally substituted heterocyclyl, optionally substituted aryl, optionally substituted heteroaryl;
each instance of R2 and R3 is independently selected from the group consisting of hydrogen, halogen, —OH, substituted hydroxyl, —SH, substituted thiol, —NH2, substituted amino, —CN, —NO2, carbonyl, silyl, sulfinyl, sulfonyl, optionally substituted alkyl, optionally substituted alkenyl, optionally substituted alkynyl, optionally substituted carbocyclyl, optionally substituted heterocyclyl, optionally substituted aryl, and optionally substituted heteroaryl; or R2 and R3 are joined to form an optionally substituted carbocyclyl, optionally substituted heterocyclyl, optionally substituted aryl, or optionally substituted heteroaryl group;
R5 is hydrogen and R6 is selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl; or R5 and R6 are joined to form a direct bond;
R7 is hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl;
R12 is hydrogen, carbonyl, silyl, optionally substituted alkyl, optionally substituted alkenyl, optionally substituted alkynyl, optionally substituted carbocyclyl, optionally substituted heterocyclyl, optionally substituted aryl, optionally substituted heteroaryl; and each instance of R13 and R14 is independently selected from the group consisting of hydrogen, halogen, —OH, substituted hydroxyl, —SH, substituted thiol, —NH2, substituted amino, —CN, —NO2, carbonyl, silyl, sulfinyl, sulfonyl, optionally substituted alkyl, optionally substituted alkenyl, optionally substituted alkynyl, optionally substituted carbocyclyl, optionally substituted heterocyclyl, optionally substituted aryl, and optionally substituted heteroaryl; or R13 and R14 are joined to form an optionally substituted carbocyclyl, optionally substituted heterocyclyl, optionally substituted aryl, or optionally substituted heteroaryl group.
30. A pharmaceutical composition comprising a combination of (a) a compound of Formula (A) or (B) and (b) one or more antineoplastic agents, wherein the compound of Formula (A) or (B) and the one or more antineoplastic agents are present in each case in free form, in the form of a pharmaceutically acceptable salt, or hydrate thereof.

### BRIEF DESCRIPTION OF THE DRAWINGS

FIG. 1. Role of Cdc7 in the G1→S Phase Transition: Schematic diagram of the initiation of DNA replication in mammalian cells. The process can be divided into several distinct phases: assembly, activation, and elongation. During assembly, the origin of DNA replication (ori) is bound by ORC1-6, the origin recognition complex that serves as a platform for pre-replication complex (pre-RC) assembly during the G1 phase of the cell cycle. This includes binding of the accessory proteins Cdc6, Cdt1, and the MCM2-7 protein complex that serves as the replicative helicase. At the G1/S phase transition, the pre-RCs are activated by both cyclin dependent kinase (CDK) and Cdc7 kinase resulting in the establishment of the replication fork. Fork establishment includes recruitment of single strand DNA binding protein (RPA) and the DNA polymerases required for leading and lagging strand synthesis. Regulation of Cdc6 and Cdt1 via phosphorylation and protein complex assembly with geminin, respectively, inhibits further assembly of functional pre-RCs ensuring genomic integrity.

FIG. 2. Elutriation profile of PhALL3.1: Centrifugal elutriation was used to synchronize the recently established Philadelphia chromosome positive acute lymphoblastic leukemia cell line (PhALL3.1). An asynchronous culture (4 liters) was pelleted, resuspended in a small volume (20 ml), and loaded into the elutriation rotor as outlined by the manufacturer (Beckman). After equilibration and washing, cell samples were eluted from the rotor based on size with the smaller cells exiting the rotor first. Ten fractions (250 ml) were taken across the gradient and standard FACS analysis performed on 10 ml of each fraction looking at DNA content. G1, S, and G2 phase cell populations are indicated in the asynchronous profile. This method allows for isolation of phase specific cell populations.

FIG. 3. Granaticin A Inhibits G1→S Transition: Granaticin A induces cell cycle arrest in G1 phase cells. Ph-ALL3.1 cells were subjected to standard centrifugal elutriation conditions and the G1 phase fraction, as determined by FACS analysis (Time Zero), was released into granaticin A (1 mM) or vehicle control (DMSO) for the indicated times. Cell samples post-release into granaticin A or vehicle control (DMSO) were pulsed for 2 hours with ³H-thymidine and samples were collected, washed, and acid precipitable counts were measured by scintillation counting. X-axis is time post-release into compound or control (in hours) and y-axis is counts per minute (CPM). The graph demonstrates that while the control cells progress through a normal S phase with a peak in incorporation as expected from the cell cycle profile observed, granaticin A treated samples stop incorporating ³H-thymidine with cell cycle profiles showing G1 phase arrest. This implies that there is a complete block to enter S phase (and activate origin firing) in the G1 population, corresponding to inhibition of Cdc7 kinase activity.

FIG. 4. Granaticin A induces Caspase 3-mediated apoptosis: 10 hours following release of G1 synchronized PhALL3.1 cells into either control (DMSO) or granaticin A, cell samples were taken and caspase 3 activity was measured using a standard fluorometric assay as described in Gao et al., “Dimeric Smac/Diablo Peptide Directly Relieves Caspase-3 Inhibition by XIAP” Journal Biological Chemistry (2007) 282:30718-30727.

FIG. 5. Granaticin A Induces Arrest and Apoptosis in S Phase cells: Granaticin A induces cell cycle arrest and apoptosis in S phase cells. Ph-ALL3.1 cells were subjected to standard centrifugal elutriation conditions and the S phase fraction, as determined by FACS analysis, was released into granaticin A (1 μM) or vehicle control (DMSO) for the indicated times and FACS analysis was performed. Displayed are FACS profiles with DNA content on the x-axis and cell number on the y-axis demonstrating cell cycle progression in the control samples and arrest in the granaticin A treated samples with subsequent apoptosis as the sub-G1 fraction increases with time. Apoptosis was confirmed in additional independent experiments. Asynchronous and time zero correspond to an asynchronous cell sample and the elutriated S phase fraction prior to release, respectively.

FIG. 6. Thymidine incorporation in S-phase: ³H-Thymidine incorporation during the release experiment outlined in the slide above. Cell samples post-release into granaticin A or vehicle control (DMSO) were pulsed for 2 hours with ³H-thymidine, and samples were collected, washed, and acid precipitable counts were measured by scintillation counting. X-axis is time post-release into compound or control (in hours) and y-axis is counts per minute (CPM). The graph demonstrates that while the control cells progress through a normal S phase with a peak in incorporation as expected from the cell cycle profile observed, granaticin A treated samples display markedly reduced incorporation early and eventually stop incorporating ³H-thymidine with cell cycle profiles showing S phase arrest. This implies that not only is late origin firing inhibited, but that replication fork progression is also inhibited by granaticin A.

FIG. 7. Granaticin A Inhibits Mcm2 phosphorylation: Following synchronization in S phase and release, PhALL3.1 cells from the experiments of FIG. 5 and FIG. 6 were collected at the indicated times post release into either control (DMSO) or granaticin A and extracts were subjected to Western blotting to detect total Mcm2 protein. This blot shows inhibition of Mcm2 phosphorylation as early as 2 hours post release into granaticin A and confirms target inhibition by granaticin A within the cell.

FIG. 8. Mcm2 phosphorylation by Cdc7: Phosphorylated forms of Mcm2 migrate faster in SDS-PAGE gels. Using highly purified human recombinant Cdc7 and Mcm2, standard in vitro kinase reactions were performed in the presence of radioactive ATP and products were separated on SDS-PAGE gels, stained with silver, and autoradiography was performed to detect phosphorylated forms of Mcm2. Control is a negative control for kinase activity, DMSO is a positive control for kinase activity, granaticin A is a small molecule inhibitor of Cdc7 kinase activity.

FIG. 9. Granaticin A Induces Cell Cycle Arrest in Hela S3 Cells: HeLa S3 cells (cervical carcinoma) were synchronized at the G1/S transition using standard double thymidine block and release. Synchronized cells (time zero) were released into media containing granaticin A for the indicated times and standard FACS analysis performed looking at DNA content. This study shows that in HeLa cells S phase progression is also blocked by exposure to granaticin A.

FIG. 10. Granaticin B Induces Cell Cycle Arrest of Hela S3 Cells: An identical experiment to

FIG. 11. Cell cycle arrest in Hela S3 cells: Morphologic appearance of control (DMSO), granaticin A, and granaticin B treated HeLa S3 cells following 15 hours release from double thymidine block. While control cells look normal and healthy, granaticin A treated cells appear arrested with flattened morphology and peri-nuclear vacuolization consistent with induction of apoptosis. This phenotype is more pronounced in the granaticin B treated cells as one now sees nuclear blebbing and cell death. The corresponding FACS profiles are shown.

FIGS. 12-14. Inhibition of Granaticin A Induced Caspase-3 Activity in HeLa cells. Growth curves in the presence of the indicated micromolar concentrations of granaticin A were performed and caspase-3 activity was measured following addition of a substrate that when cleaved by caspase-3 results in light emission which is quantitated by a fluorimeter (y-axis) as a function of time (x-axis). FIG. 12: HeLa cells alone; FIG. 13: Bcl-XL overexpressing HeLa cells; and FIG. 14: Bcl-XL overexpressing HeLa cells in the presence of 10 micromolar Ac-DEVD-CHO (a caspase-3 peptide aldehyde inhibitor) demonstrating activation of caspase-3 and apoptosis in granaticin A treated HeLa cells that is rescued by both overexpression of Bcl-XL and chemical inhibition of caspase-3 activity.

FIG. 15. Granaticin A Does Not Activate the S Phase Checkpoint in Hela Cells: Exposure of HeLa cells to granaticin A does not activate the S phase checkpoint and results in increased double strand DNA breaks. HeLa cells were incubated in the presence of granaticin A or carrier control (DMSO) for the indicated times and whole cell extracts were subjected to SDS-PAGE and western blot analysis. Asyn=asynchronously growing HeLa cells. HU=HeLa cells exposed to hydroxyurea for the indicated time. HU was used as a control for DNA damaging agent that is known to inhibit the DNA damage checkpoint response. Again, we observed that Mcm2 mobility is shifted in the cells treated with granaticin A. MCM2 mobility is not affected by HU. Activation of the DNA damage/replication checkpoint correlates with ATM/ATR dependent phosphorylation of Chk1 at Ser345. ChK1 Ser345 levels are below detection limits, suggesting that with Cdc7 inhibition, a DNA replication checkpoint is either not efficiently activated or maintained and that inhibitory signals preventing cells from progressing in the cell cycle might not be generated.

FIGS. 16A-16B. Granaticin A and Granaticin B inhibit multiple tumor types: The indicated cell lines and primary patient samples were tested against the compounds listed in standard cytotoxicity assays (12 point dose response using Alamar blue reduction as an indicator of cell viability). Assays were done in triplicate and IC50 determinations were run in 12-point serial dilutions from 10 mM to 5 nM. IC50 values are displayed with darker shades indicating low nanomolar values and lighter shades high micromolar values, respectively. FIGS. 16A and 16B summarize the IC50 determinations for granaticin A and granaticin B, as well as Derivatives 1 and 2 (FIG. 16A) against multiple non-small cell lung cancer cell lines, ovarian cancer cell lines, mesothelioma, sarcoma, and melanoma cell lines including cells isolated from primary patient ocular melanoma samples (124859-A and-SB). These include ras and EGFR gatekeeper mutation containing cell lines in addition to p53 mutant cell lines and other known chemotherapy resistant cell lines. Specifically, this included multi-drug resistance phenotype (MDR-overexpressing) cell lines from multiple hematologic and solid tumor cell lines. Table 2 provided herein summarizes the data provided in FIG. 16B (see Examples). Table 1 highlights a series of primary patient leukemia samples-both acute and chronic and treatment naïve and refractory tested against granaticins A and B and Derivatives 1 and 2 (see Examples). FIGS. 16A and 16B, and Tables 1-2, together, show that granaticin A, Derivatives 1 and 2, and especially granaticin B, are pan-active against multiple tumor cell lines and in primary patient samples from patients with chronic and acute leukemias both treatment naïve and chemotherapy refractory.

FIG. 17. t(9:22)(q34;q11) Acute Lymphocytic Leukemia cell line: Based on the results of the cytotoxicity assays, granaticin A and granaticin B were chosen to pursue studies in mice using the Ph-ALL3.1 mouse model. The PhALL3.1 cell line was retrovirally transduced with a GFP-firefly luciferase expressing construct using standard techniques. The infected cells were sorted by FACS for CD19 and GFP co-expression. Panel A is the FACS profile of the cells before transduction and Panel B is the FACS profile of the cells after transduction demonstrating CD19-GFP double positive cells which were then used to inject immunocompromised SCID-Beige mice. Ph+ ALL cells were isolated from the pleural fluid of a patient treated on the Leukemia Service, and used to create a cell line that grows readily in culture. The cytogenetics were confirmed by karyotype, FISH, RT-PCR, and Western blot analysis for p190 bcr-abl. The cell line was then transduced with a retrovirus GFP-firefly luciferase construct. Granaticin A has been shown to be very active against this cell line in the cell based assays.

FIGS. 18A-18B. Mouse survival following tumor cell inoculation: 5 million PhALL3.1 tumor cells were injected into the tail veins of a cohort of eight SCID-Beige immunocompromised mice and disease progression was monitored on the indicated days using in vivo bioluminescence imaging. FIG. 18A shows that this mouse model of human acute lymphoblastic leukemia faithfully duplicates the progression seen in human disease. FIG. 18B shows that when left untreated these mice meet criteria for euthanasia by day 41 indicating a rapid, progressive, and lethal leukemia.

FIGS. 19A-19B. Granaticin A Inhibits growth of PhALL3. 1 in vivo: Using a SCID-Beige mouse intraperitoneal tumor model of PhALL3.1, mice were intraperitoneally treated with vehicle control (DMSO) or the indicated doses of granaticin A on days 3, 6, 9, and 12. Day 16 in vivo bioluminescence imaging (FIG. 19A) and quantitation of tumor volume are shown (FIG. 19B) and indicate 90% reduction in tumor volume by Day 16 in mice treated at the highest dose (3.0 mg/kg). Separate necropsy data revealed no significant (grade 2 or higher) organ toxicity in all mice examined at each dose level.

FIGS. 20A-20C. Granaticin B Inhibits Ph-ALL3.1 growth in vivo: Given the rapid intravenous clearance of granaticin A by the liver, granaticin B was used in a continuous infusion experiment with the PhALL3.1 animal model. Diffusion pumps containing granaticin B were surgically implanted in the flanks of 5 cohorts of mice on day +3 after injection of 5 million cells via the tail veins, and the drug (or control—C-DMSO) at the indicated concentration was allowed to diffuse for seven days before pump removal. The mice were imaged on day 12 (FIGS. 20A-20B) and tumor volume remaining was calculated (FIG. 20C). 95% tumor reduction was seen at the highest dose. The mice tolerated the procedure without obvious organ toxicity as seen on necropsy and indicate that granaticin B is stable enough when given intravenously to achieve efficacy against this lethal tumor model.

FIG. 21. Granaticin B stability study comparison in HP/MP: Standard plasma stability studies were conducted with Granaticin B. The recovery of Granaticin B was measured by mass spectrometry.

FIG. 22A-22C. Granaticin B is Effective Against Multiple Solid Tumors: granaticin B is efficacious against mouse models of melanoma (FIG. 22A), non-small cell lung cancer (FIG. 22B), and ovarian cancer (FIG. 22C). For the ovarian cancer experiments, SCID-Beige mice were injected with 5 million OVCAR3 cells that had been transduced with a GFP-firefly luciferase expressing plasmid (Frattini, M. G. and Brentjens, R. J., unpublished data). These cells were then injected intraperitoneally into 4 cohorts of mice on Day 1. From days 7-14 the mice were injected daily with the indicated concentrations of granaticin B in the intraperitoneal space. Imaging (luciferase activity) was acquired on day 42 and revealed essentially no disease in the mice treated at 3 mg/kg granaticin B. For the melanoma and non small-cell lung cancer models, SKMEL-28 and A459 cells, respectively were used in standard nude mouse xenograft experiments. Nude mice were treated with 2.5 mg/kg/day granaticin B in a continuous fashion using osmotic diffusion pumps. Sections of the treated xenografts are shown after euthanasia at day 28 and H&E staining. Control sections revealed no evidence of necrosis or cell death (not shown).

FIGS. 23A-23C. Granaticin A and B are not cytotoxic to normal diploid cells: FIGS. 23A-23B: Granaticin B exposure is not cytotoxic to normal diploid cells. Either HeLa or RPE (retinal pigment epithelial cells, a normal human diploid cell line) cells were incubated in carrier control (DMSO) or granaticin B (1 microM) for 24 hours and samples were subjected to standard FACS analysis with the x-axis indicating DNA content (FIG. 23A). The cell cycle profile for the RPE cells is essentially unaffected by granaticin B exposure where the HeLa cell cycle profile indicates apoptotic cell death with an increase in sub-G1 DNA containing cells and loss of normal cell cycle progression. Extracts from cells at the indicated times, exposed to either DMSO or granaticin B (1 microM), were used in standard caspase-3 activity assays to monitor for induction of apoptosis and show that capsae-3 is only activated in HeLa cells exposed to granaticin B (FIG. 23B). Granaticin B exposure does not result in caspase-3 activation in RPE cells. FIG. 23C: Granaticin A exposure is not cytotoxic to normal diploid cells. Either HeLa or RPE (retinal pigment epithelial cells, a normal diploid cell line) cells were incubated in carrier control (DMSO) or granaticin A for 24 hours and samples were subjected to standard FACS analysis with FL2 indicating DNA content. The cell cycle profile for the RPE cells is unaffected by granaticin A exposure where the HeLa cell cycle profile indicates apoptotic cell death with an increase in sub-G1 DNA containing cells.

FIGS. 24A-24B. Granaticin B is very effective against JOKE-2 cells. The JOKE-2 cell line was derived from a patient with Philadelphia chromosome positive Acute Lymphoblastic Leukemia (Ph-ALL). The patient was clinically resistant to imatinib mesylate and the Bcr-Abl kinase was found to have the following high-risk resistance mutations in the Abl kinase domain (F317L, F359V, T3151, and E255K). FIG. 24A: Joke-2 cells were grown in the presence of DMSO control or granaticin B (1 microM) for the indicated times and viability was measured after staining a cell sample in trypan blue (Sigma-Aldrich). FIG. 24B: Extracts from cells at the indicated times, exposed to either DMSO or granaticin B (1 microM), were used in standard caspase-3 activity assays to monitor for induction of apoptosis and show that capsae-3 is only activated in cells exposed to granaticin B.

FIG. 25A-25B. Triple treatment of LBS-007, Venetoclax and Azacytidine is surprisingly effective in p53 mutant Kasumi-1 AML cell line. 25A: Kasumi-1 cells were grown in presence of DMSO (Ctrl) or LBS-007 (40 nM), Venetoclax (20 nM) or Azacytidine (500 nM). Single treatment of LBS-007 and Venetoclax significantly decreased cell viability when compared to control. 25B: Kasumi-1 cells were grown in presence of DMSO (Ctrl) or double combination of 007+Ven, 007+Aza, Ven+Aza or a triple combination of 007+Ven+Aza. The double and triple treatments all significantly decreased cell viability when compared to control. Importantly, triple treatment significantly decreases cell viability when compared to double treatments, indicating that triple treatment has superior efficacy compared to double treatments.

FIG. 26A-26B. Triple treatment of LBS-007, Venetoclax and Azacytidine is surprisingly effective in p53 wild type CESS AML cell line. 26A: CESS cells were grown in presence of DMSO (Ctrl) or LBS-007 (40 nM), Venetoclax (20 nM) or Azacytidine (500 nM) as a single agent. Single treatment of LBS-007 significantly decreased cell viability when compared to control. 26B: CESS cells were grown in presence of DMSO (Ctrl) or double combination of 007+Ven, 007+Aza, Ven+Aza or a triple combination of 007+Ven+Aza. The double and triple treatments all significantly decreased cell viability when compared to control. Importantly, triple treatment significantly decreases cell viability when compared to double treatments, indicating that triple treatment has superior efficacy compared to double treatments.

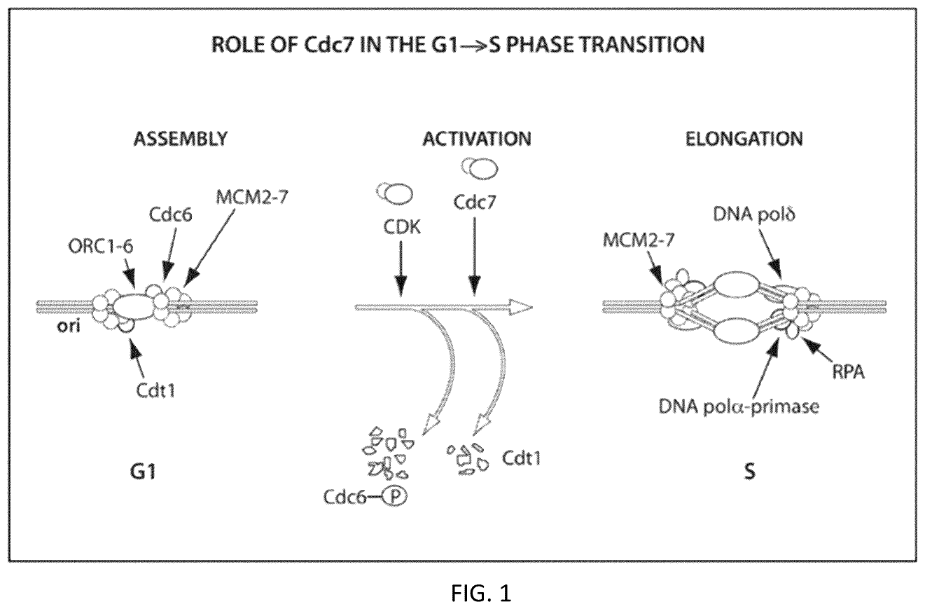

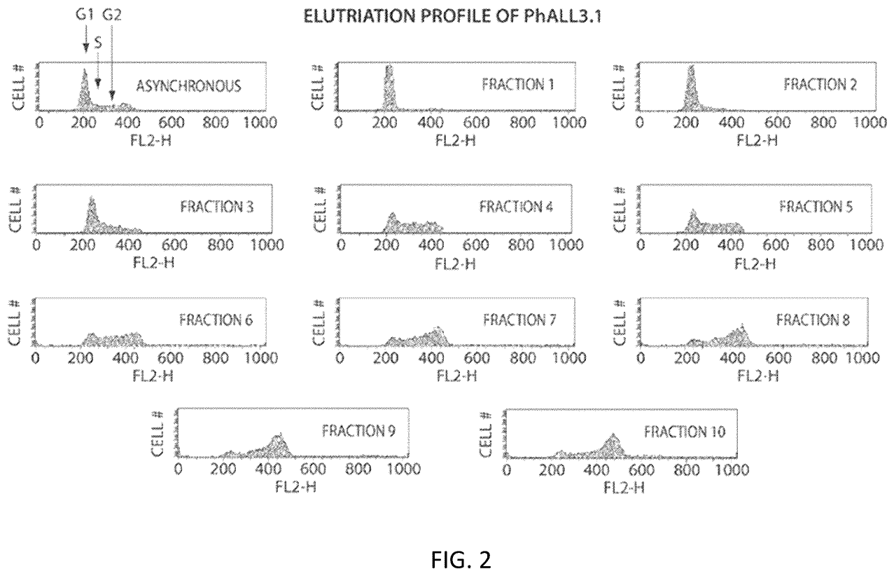

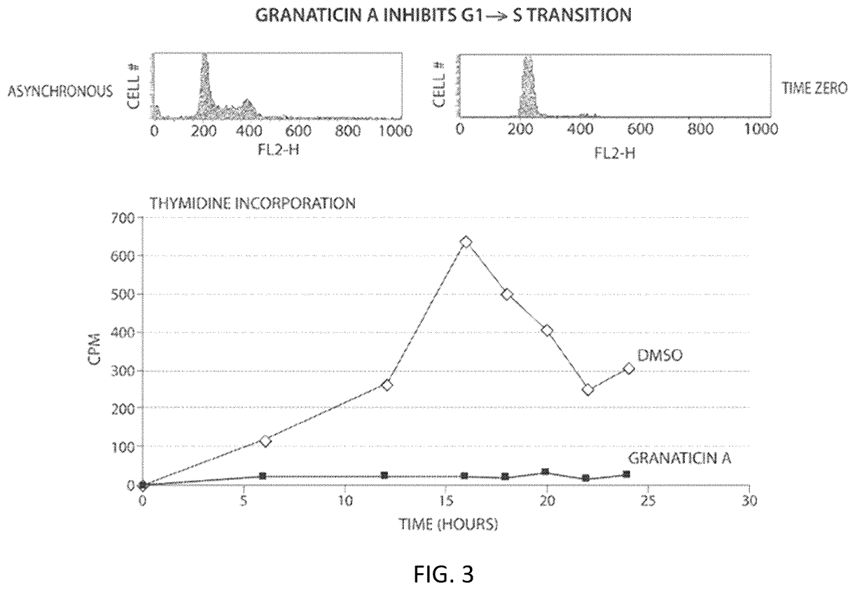

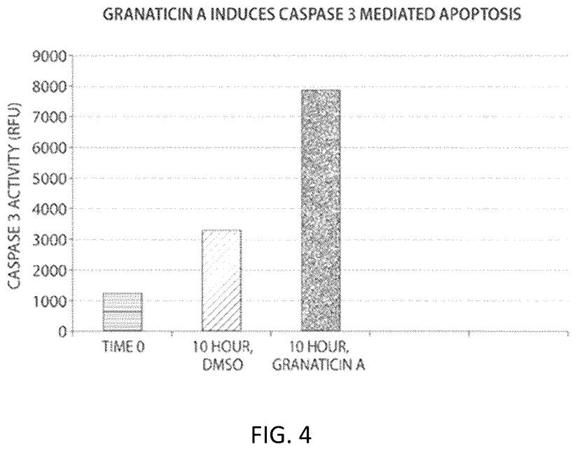

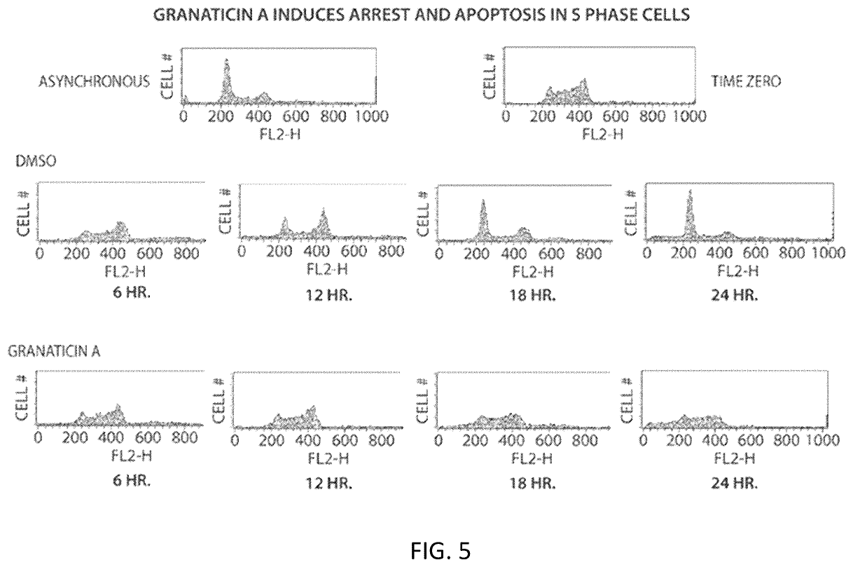

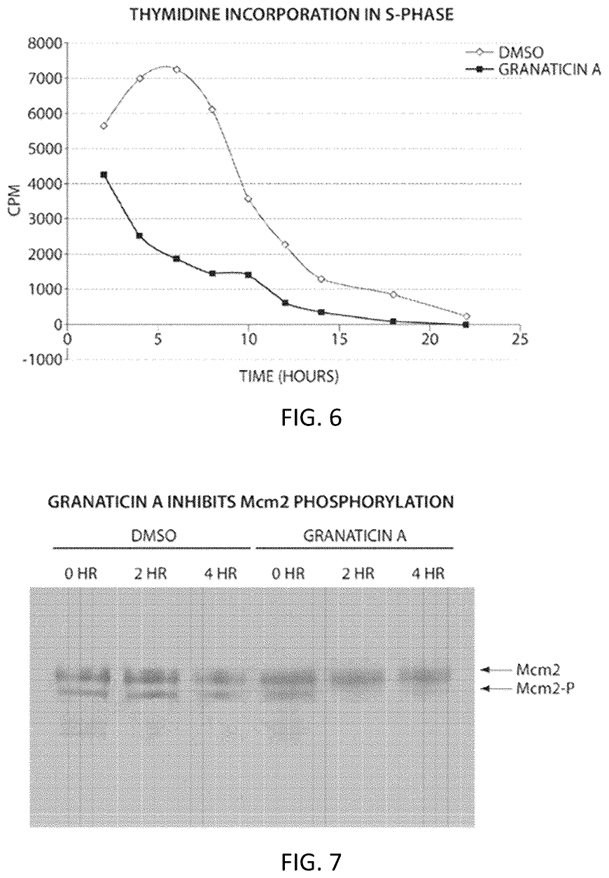

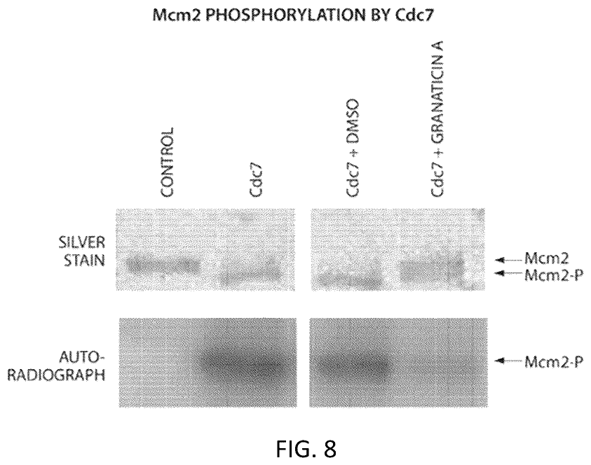

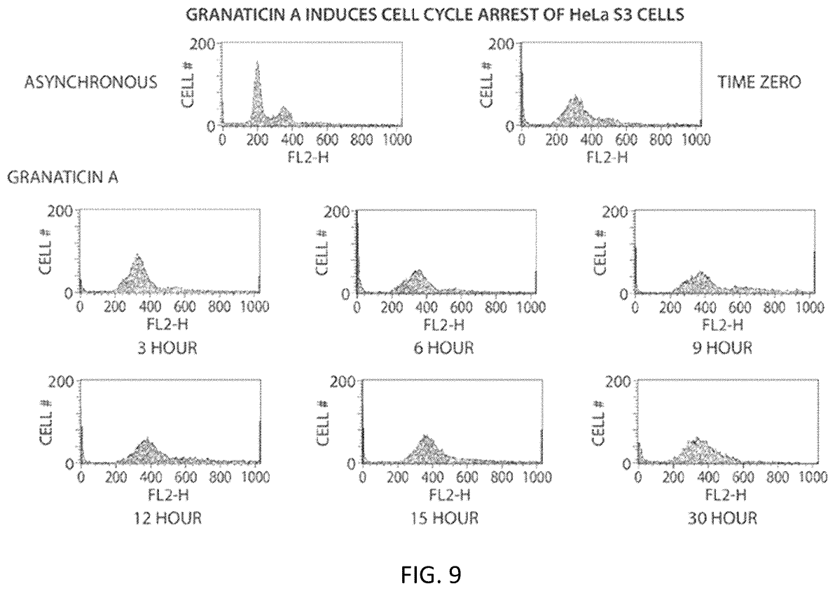

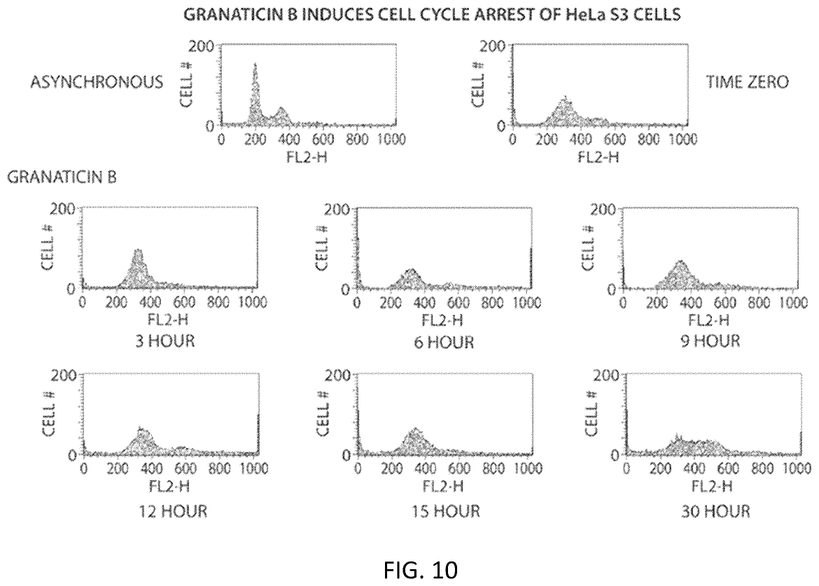

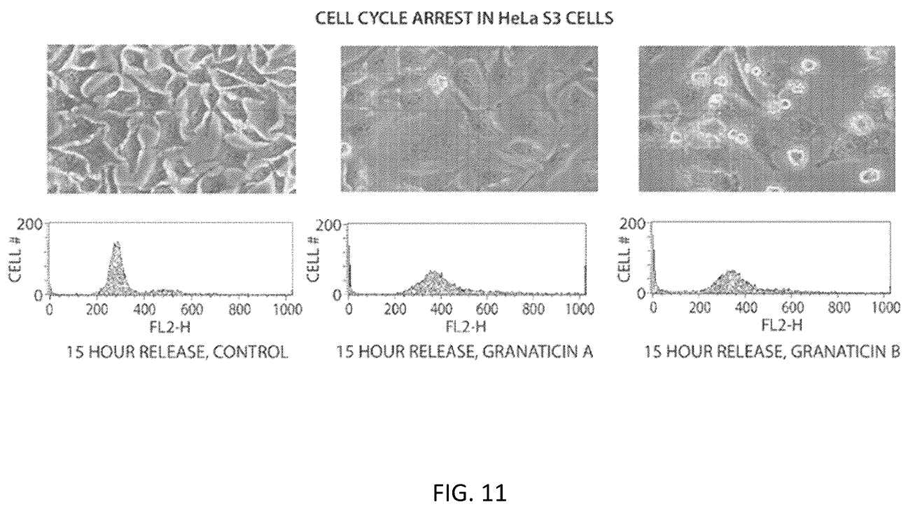

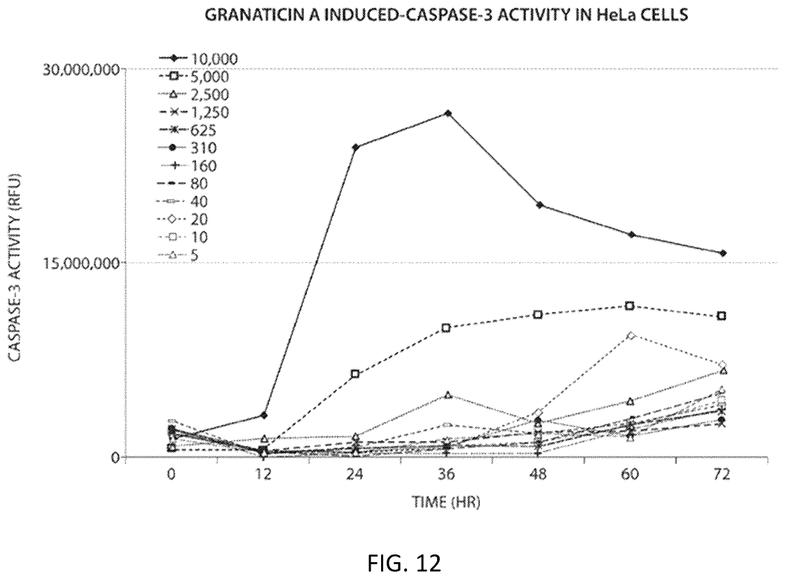

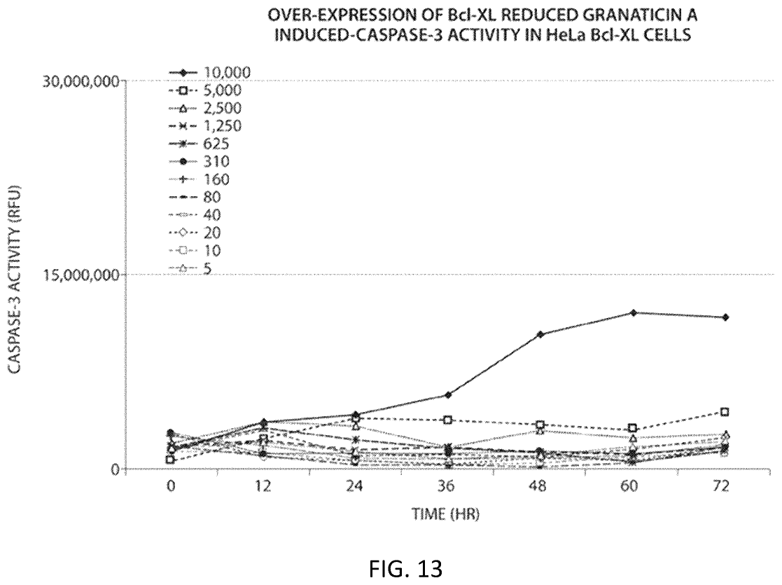

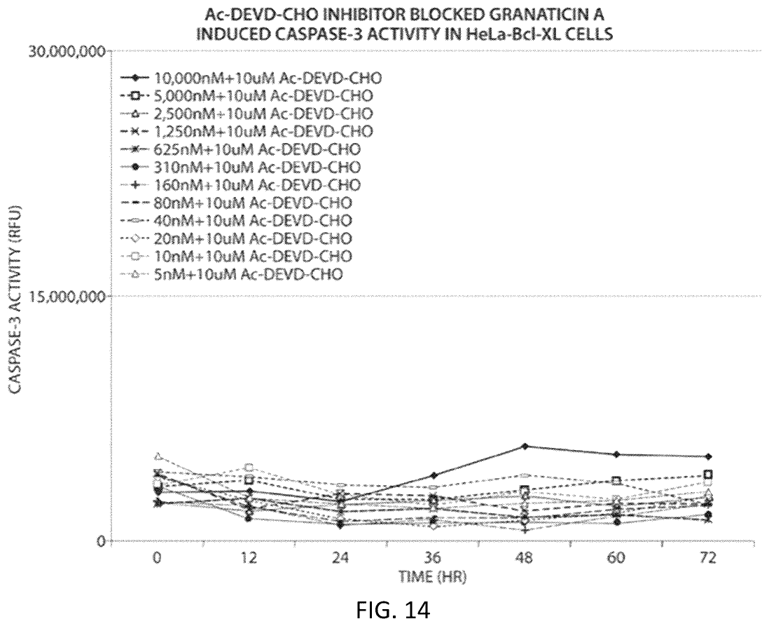

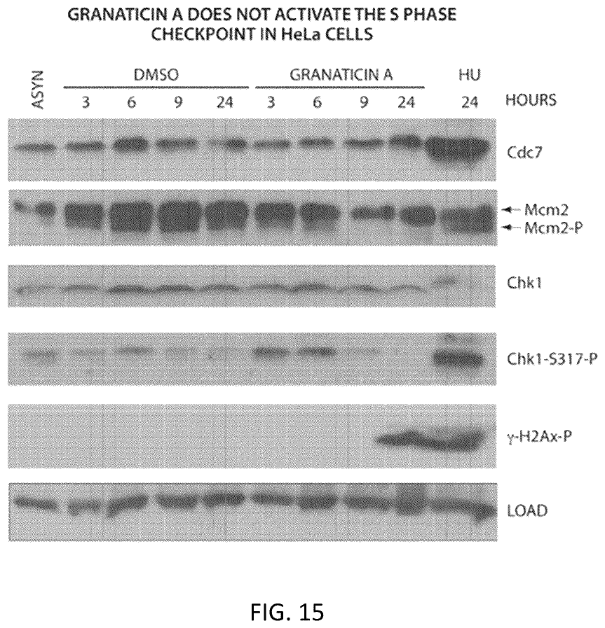

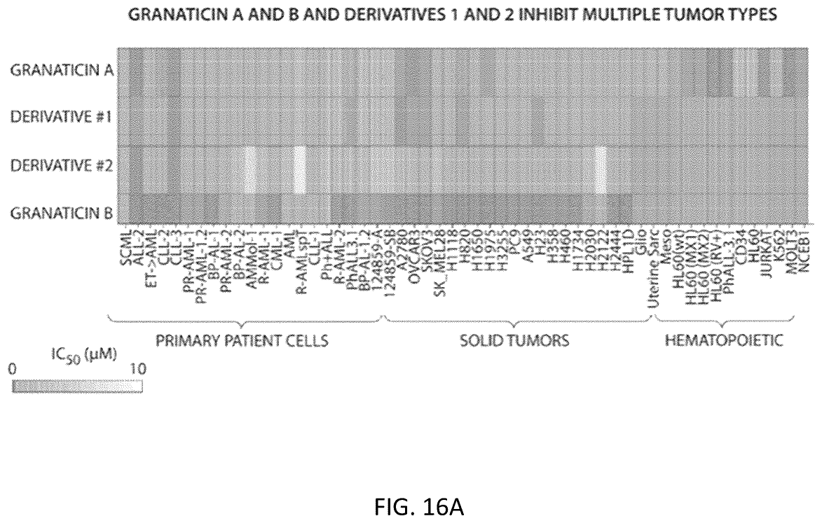

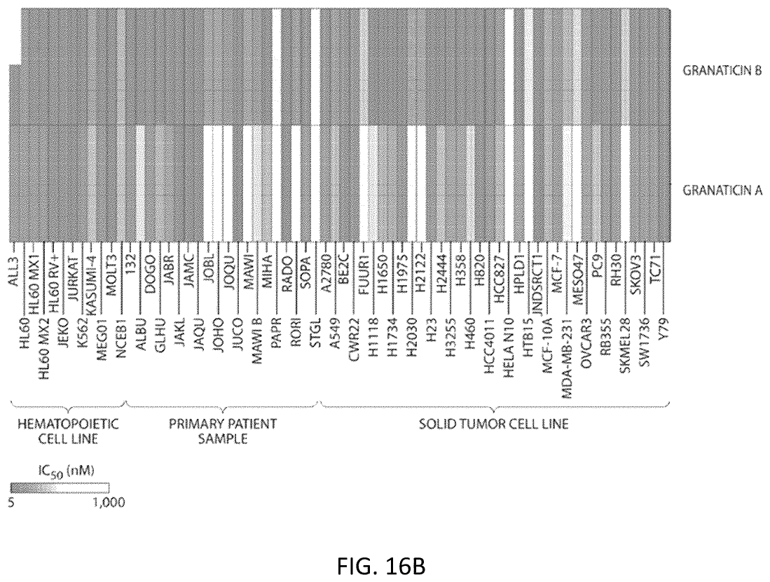

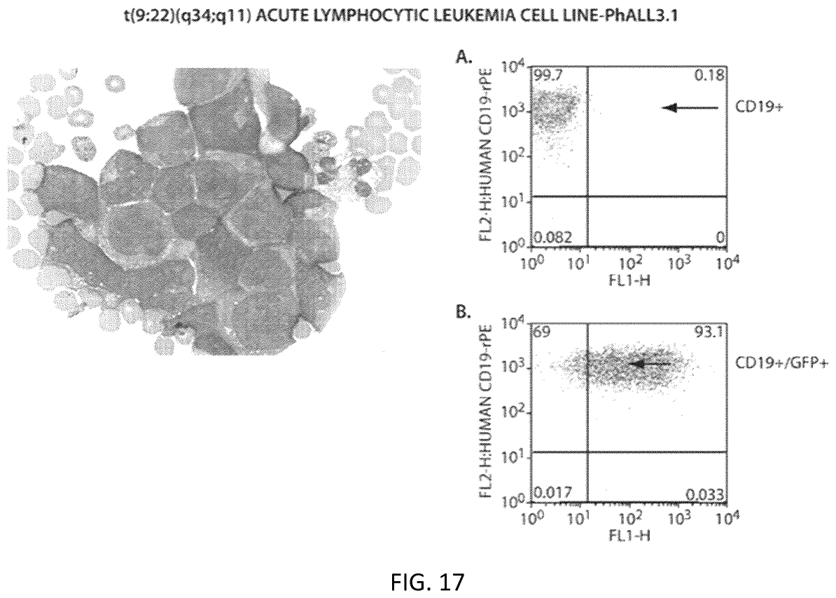

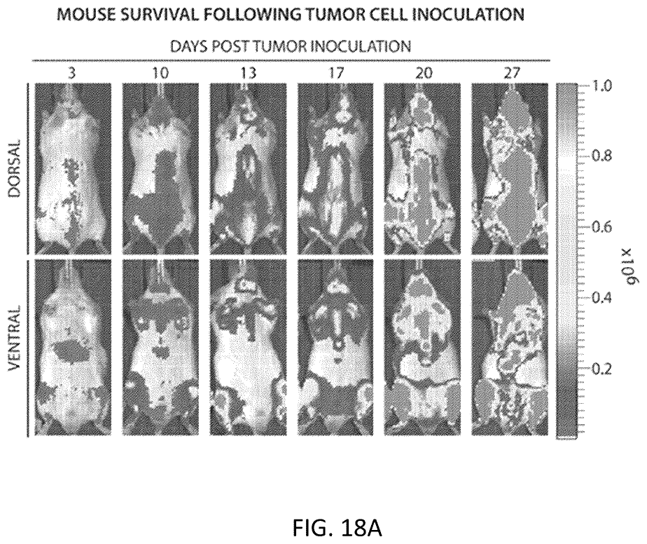

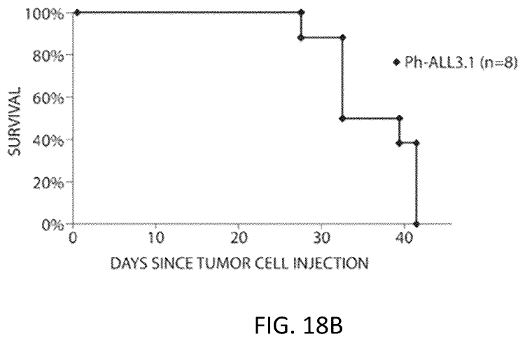

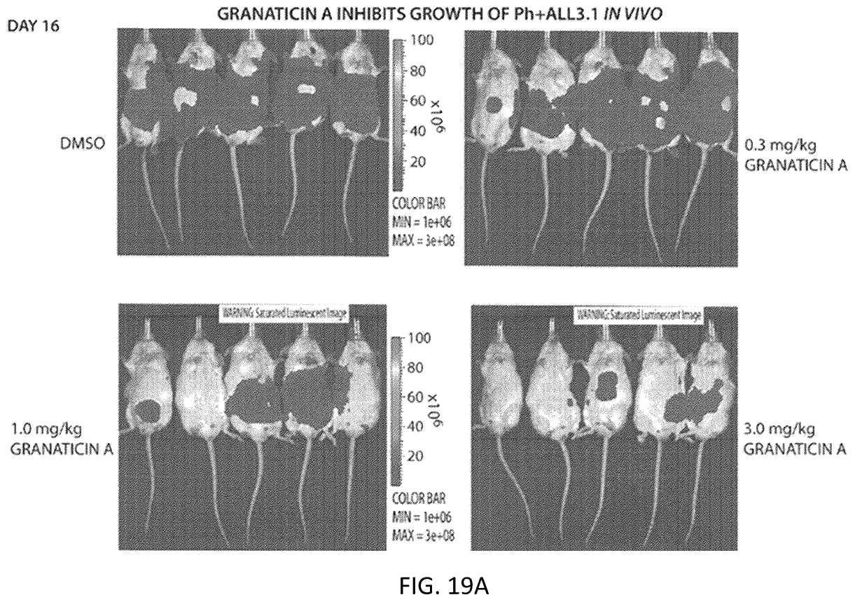

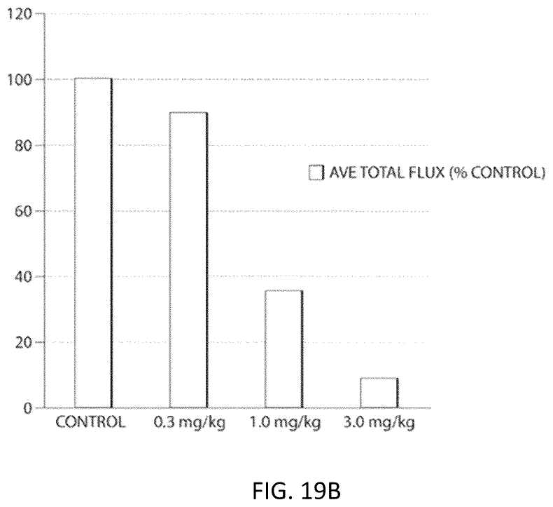

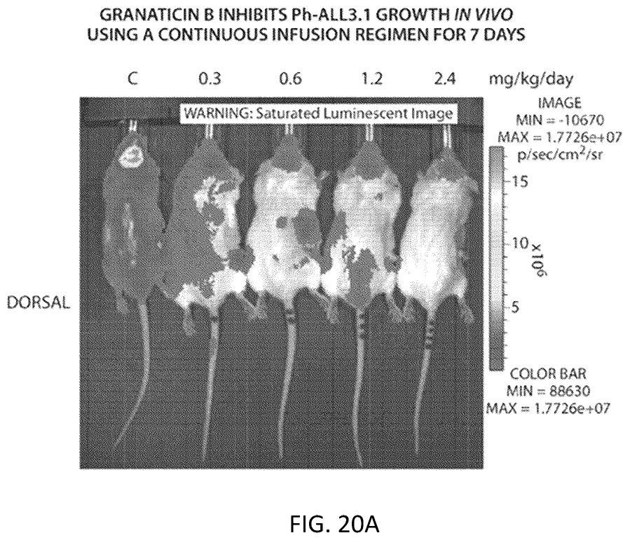

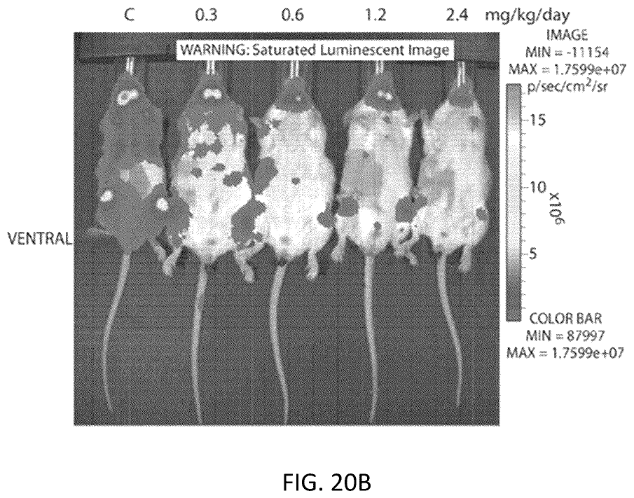

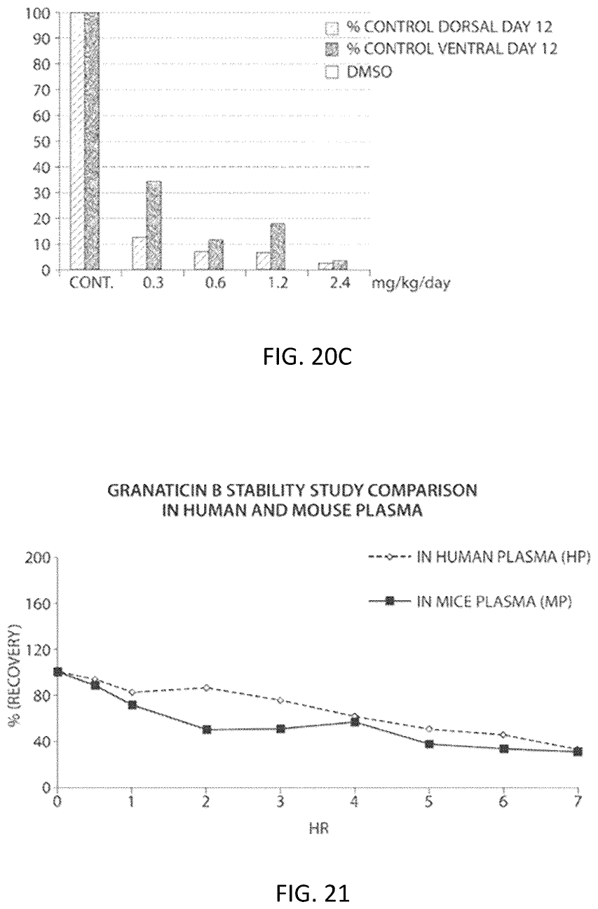

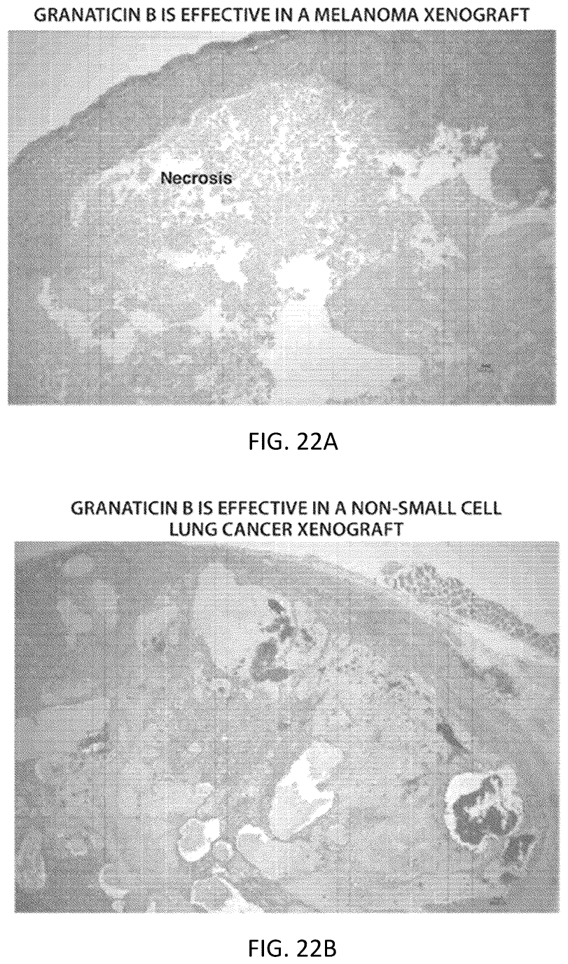

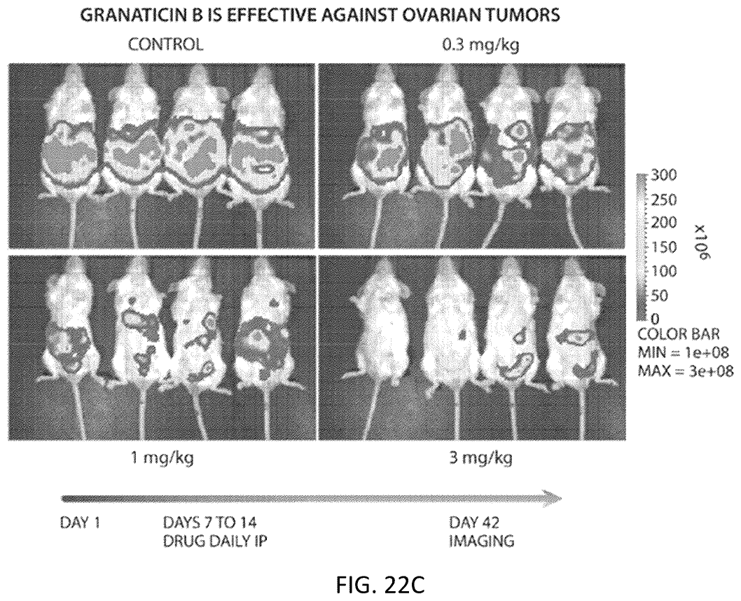

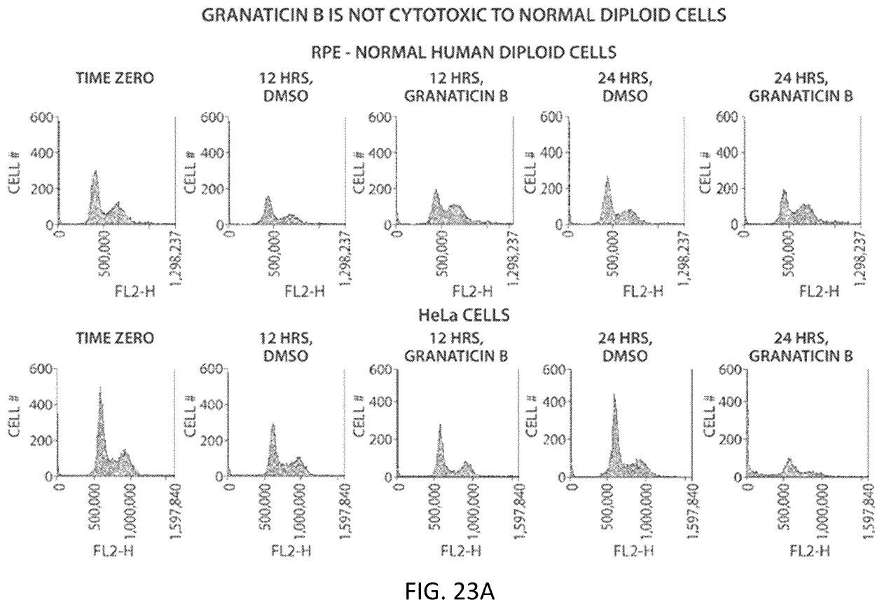

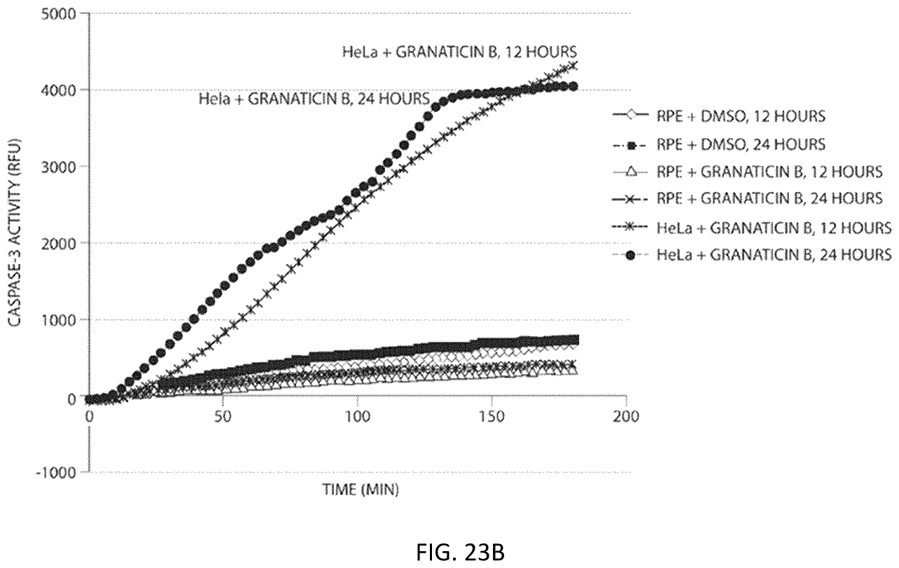

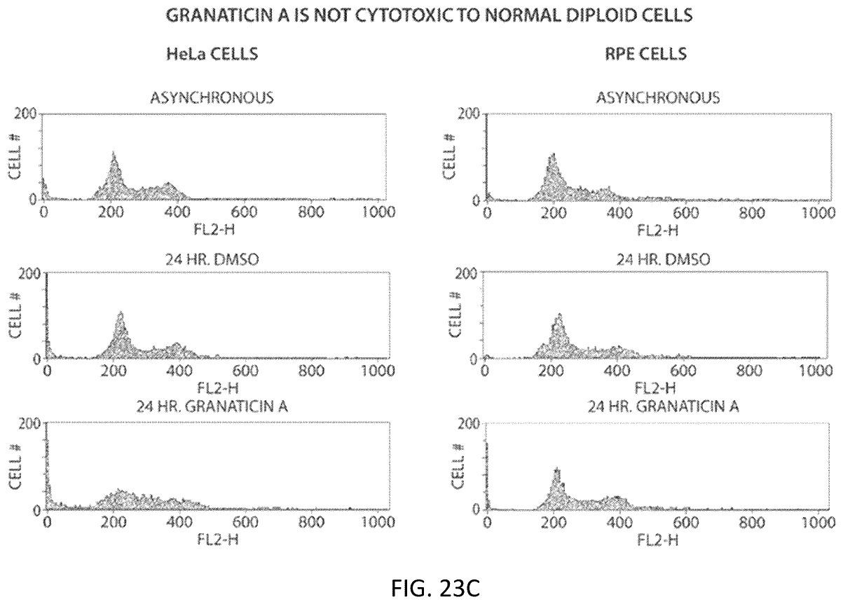

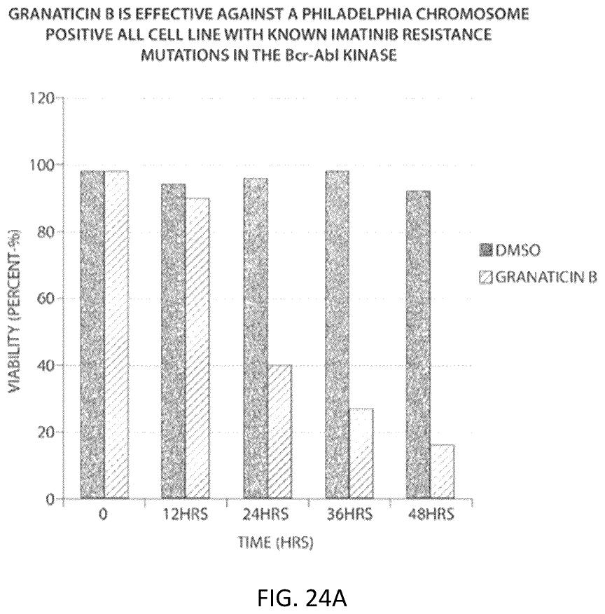

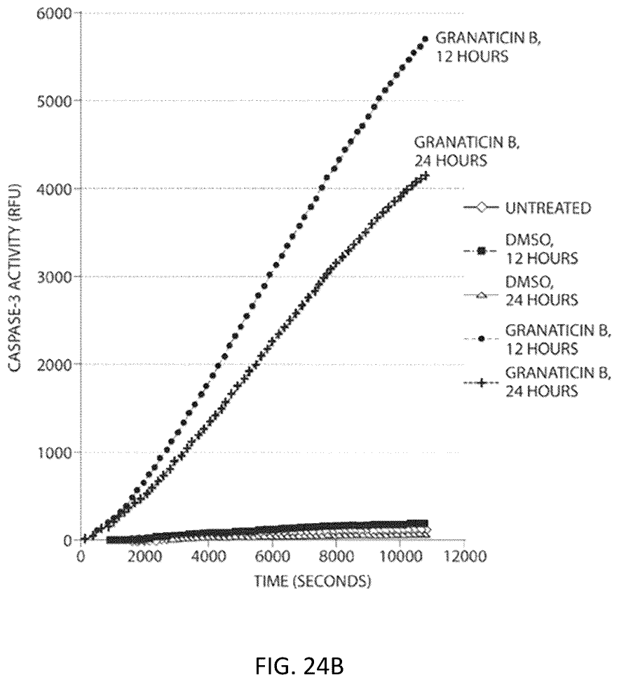

### DEFINITIONS

The compositions of the present invention can comprise, consist of, or consist essentially of the essential elements and limitations of the invention described herein, as well as any of the additional or optional ingredients, components, or limitations described herein.

As used in the specification and claims, the singular form “a” “an” and “the” include plural references unless the context clearly dictates otherwise. For example, the term “a” cell includes a plurality of cells, including mixtures thereof.

“About” in the context of amount values refers to an average deviation of maximum ±20%, preferably ±10% or more preferably ±5% based on the indicated value. For example, an amount of about 30 mol % anionic lipid refers to 30 mol % ±6 mol %, preferably 30 mol % ±3 mol % or more preferably 30 mol % ±1.5 mol % anionic lipid with respect to the total lipid/amphiphile molarity.

Definitions of specific functional groups and chemical terms are described in more detail below. The chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito, 1999; Smith and March March's Advanced Organic Chemistry, 5^{th} Edition, John Wiley & Sons, Inc., New York, 2001; Larock, Comprehensive Organic Transformations, VCH Publishers, Inc., New York, 1989; and Carruthers, Some Modern Methods of Organic Synthesis, 3^{rd} Edition, Cambridge University Press, Cambridge, 1987.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high pressure liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, E. L. Stereochemistry of Carbon Compounds (McGraw-Hill, NY, 1962); and Wilen, S. H. Tables of Resolving Agents and Optical Resolutions p. 268 (E. L. Eliel, Ed., Univ. of Notre Dame Press, Notre Dame, Ind. 1972). The invention additionally encompasses compounds as individual isomers substantially free of other isomers, and alternatively, as mixtures of various isomers.

When a range of values is listed, it is intended to encompass each value and sub-range within the range. For example “C₁₋₆ alkyl” is intended to encompass, C₁, C₂, C₃, C₄, C₅, C₆, C₁₋₆, C₁₋₅, C₁₋₄, C₁₋₃, C₁₋₂, C₂₋₆, C₂₋₅, C₂₋₄, C₂₋₃, C₃₋₆, C₃₋₅, C₃₋₄, C₄₋₆, C₄₋₅, and C₅₋₆ alkyl. As used herein, “alkyl” refers to a radical of a straight-chain or branched saturated hydrocarbon group having from 1 to 10 carbon atoms (“C₁₋₁₀ alkyl”). In some embodiments, an alkyl group has 1 to 9 carbon atoms (“C₁₋₉ alkyl”). In some embodiments, an alkyl group has 1 to 8 carbon atoms (“C₁₋₈ alkyl”). In some embodiments, an alkyl group has 1 to 7 carbon atoms (“C₁₋₇ alkyl”). In some embodiments, an alkyl group has 1 to 6 carbon atoms (“C₁₋₆ alkyl”). In some embodiments, an alkyl group has 1 to 5 carbon atoms (“C₁₋₅ alkyl”). In some embodiments, an alkyl group has 1 to 4 carbon atoms (“C₁₋₄ alkyl”). In some embodiments, an alkyl group has 1 to 3 carbon atoms (“C₁₋₃ alkyl”). In some embodiments, an alkyl group has 1 to 2 carbon atoms (“C₁₋₂ alkyl”). In some embodiments, an alkyl group has 1 carbon atom (“C₁ alkyl”). In some embodiments, an alkyl group has 2 to 6 carbon atoms (“C₂₋₆ alkyl”). Examples of C₁₋₆ alkyl groups include methyl (C₁), ethyl (C₂), n-propyl (C₃), isopropyl (C₃), n-butyl (C₄), tert-butyl (C₄), sec-butyl (C₄), iso-butyl (C₄), n-pentyl (C₅), 3-pentanyl (C₅), amyl (C₅), neopentyl (C₅), 3-methyl-2-butanyl (C₅), tertiary amyl (C₅), and n-hexyl (C₆). Additional examples of alkyl groups include n-heptyl (C₇), n-octyl (C₈) and the like. Unless otherwise specified, each instance of an alkyl group is independently unsubstituted (an “unsubstituted alkyl”) or substituted (a “substituted alkyl”) with one or more substituents. In certain embodiments, the alkyl group is an unsubstituted C₁₋₁₀ alkyl (e.g., —CH₃). In certain embodiments, the alkyl group is a substituted C₁₋₁₀ alkyl.

“Perhaloalkyl” is a substituted alkyl group as defined herein wherein all of the hydrogen atoms are independently replaced by a halogen, e.g., fluoro, bromo, chloro, or iodo. In some embodiments, the alkyl moiety has 1 to 8 carbon atoms (“C₁₋₈ perhaloalkyl”). In some embodiments, the alkyl moiety has 1 to 6 carbon atoms (“C₁₋₆ perhaloalkyl”). In some embodiments, the alkyl moiety has 1 to 4 carbon atoms (“C₁₋₄ perhaloalkyl”). In some embodiments, the alkyl moiety has 1 to 3 carbon atoms (“C₁₋₃ perhaloalkyl”). In some embodiments, the alkyl moiety has 1 to 2 carbon atoms (“C₁₋₂ perhaloalkyl”). In some embodiments, all of the hydrogen atoms are replaced with fluoro. In some embodiments, all of the hydrogen atoms are replaced with chloro. Examples of perhaloalkyl groups include —CF₃, —CF₂CF₃, —CF₂CF₂CF₃, —CCl₃, —CFCl₂, —CF₂Cl, and the like.

As used herein, “alkenyl” refers to a radical of a straight-chain or branched hydrocarbon group having from 2 to 10 carbon atoms and one or more carbon-carbon double bonds (“C₂₋₁₀ alkenyl”). In some embodiments, an alkenyl group has 2 to 9 carbon atoms (“C₂₋₉ alkenyl”). In some embodiments, an alkenyl group has 2 to 8 carbon atoms (“C₂₋₈ alkenyl”). In some embodiments, an alkenyl group has 2 to 7 carbon atoms (“C₂₋₇ alkenyl”). In some embodiments, an alkenyl group has 2 to 6 carbon atoms (“C₂₋₆ alkenyl”). In some embodiments, an alkenyl group has 2 to 5 carbon atoms (“C₂₋₅ alkenyl”). In some embodiments, an alkenyl group has 2 to 4 carbon atoms (“C₂₋₄ alkenyl”). In some embodiments, an alkenyl group has 2 to 3 carbon atoms (“C₂₋₃ alkenyl”). In some embodiments, an alkenyl group has 2 carbon atoms (“C₂ alkenyl”). The one or more carbon-carbon double bonds can be internal (such as in 2-butenyl) or terminal (such as in 1-butenyl). Examples of C₂₋₄ alkenyl groups include ethenyl (C₂), 1-propenyl (C₃), 2-propenyl (C₃), 1-butenyl (C₄), 2-butenyl (C₄), butadienyl (C₄), and the like. Examples of C₂₋₆ alkenyl groups include the aforementioned C₂₋₄ alkenyl groups as well as pentenyl (C₅), pentadienyl (C₅), hexenyl (C₆), and the like. Additional examples of alkenyl include heptenyl (C₇), octenyl (C₈), octatrienyl (C₈), and the like. Unless otherwise specified, each instance of an alkenyl group is independently unsubstituted (an “unsubstituted alkenyl”) or substituted (a “substituted alkenyl”) with one or more substituents. In certain embodiments, the alkenyl group is an unsubstituted C₂₋₁₀ alkenyl. In certain embodiments, the alkenyl group is a substituted C₂₋₁₀ alkenyl.

As used herein, “alkynyl” refers to a radical of a straight-chain or branched hydrocarbon group having from 2 to 10 carbon atoms and one or more carbon-carbon triple bonds (“C₂₋₁₀ alkynyl”). In some embodiments, an alkynyl group has 2 to 9 carbon atoms (“C₂₋₉ alkynyl”). In some embodiments, an alkynyl group has 2 to 8 carbon atoms (“C₂₋₈ alkynyl”). In some embodiments, an alkynyl group has 2 to 7 carbon atoms (“C₂₋₇ alkynyl”). In some embodiments, an alkynyl group has 2 to 6 carbon atoms (“C₂₋₆ alkynyl”). In some embodiments, an alkynyl group has 2 to 5 carbon atoms (“C₂₋₅ alkynyl”). In some embodiments, an alkynyl group has 2 to 4 carbon atoms (“C₂₋₄ alkynyl”). In some embodiments, an alkynyl group has 2 to 3 carbon atoms (“C₂₋₃ alkynyl”). In some embodiments, an alkynyl group has 2 carbon atoms (“C₂ alkynyl”). The one or more carbon-carbon triple bonds can be internal (such as in 2-butynyl) or terminal (such as in 1-butynyl). Examples of C₂₋₄ alkynyl groups include, without limitation, ethynyl (C₂), 1-propynyl (C₃), 2-propynyl (C₃), 1-butynyl (C₄), 2-butynyl (C₄), and the like. Examples of C₂₋₆ alkenyl groups include the aforementioned C₂₋₄ alkynyl groups as well as pentynyl (C₅), hexynyl (C₆), and the like. Additional examples of alkynyl include heptynyl (C₇), octynyl (C₈), and the like. Unless otherwise specified, each instance of an alkynyl group is independently unsubstituted (an “unsubstituted alkynyl”) or substituted (a “substituted alkynyl”) with one or more substituents. In certain embodiments, the alkynyl group is an unsubstituted C₂₋₁₀ alkynyl. In certain embodiments, the alkynyl group is a substituted C₂₋₁₀ alkynyl.

As used herein, “carbocyclyl” refers to a radical of a non-aromatic cyclic hydrocarbon group having from 3 to 10 ring carbon atoms (“C₃₋₁₀ carbocyclyl”) and zero heteroatoms in the non-aromatic ring system. In some embodiments, a carbocyclyl group has 3 to 8 ring carbon atoms (“C₃₋₈ carbocyclyl”). In some embodiments, a carbocyclyl group has 3 to 6 ring carbon atoms (“C₃₋₆ carbocyclyl”). In some embodiments, a carbocyclyl group has 3 to 6 ring carbon atoms (“C₃₋₆ carbocyclyl”). In some embodiments, a carbocyclyl group has 5 to 10 ring carbon atoms (“C₅₋₁₀ carbocyclyl”). Exemplary C₃₋₆ carbocyclyl groups include, without limitation, cyclopropyl (C₃), cyclopropenyl (C₃), cyclobutyl (C₄), cyclobutenyl (C₄), cyclopentyl (C₅), cyclopentenyl (C₅), cyclohexyl (C₆), cyclohexenyl (C₆), cyclohexadienyl (C₆), and the like. Exemplary C₃₋₈ carbocyclyl groups include, without limitation, the aforementioned C₃₋₆ carbocyclyl groups as well as cycloheptyl (C₇), cycloheptenyl (C₇), cycloheptadienyl (C₇), cycloheptatrienyl (C₇), cyclooctyl (C₅), cyclooctenyl (C₈), bicyclo[2.2.1]heptanyl (C₇), bicyclo[2.2.2]octanyl (C₈), and the like. Exemplary C₃₋₁₀ carbocyclyl groups include, without limitation, the aforementioned C₃₋₈ carbocyclyl groups as well as cyclononyl (C₉), cyclononenyl (C₉), cyclodecyl (C₁₀), cyclodecenyl (C₁₀), octahydro-1H-indenyl (C₉), decahydronaphthalenyl (C₁₀), spiro[4.5]decanyl (C₁₀), and the like. As the foregoing examples illustrate, in certain embodiments, the carbocyclyl group is either monocyclic (“monocyclic carbocyclyl”) or polycyclic (e.g., containing a fused, bridged or spiro ring system such as a bicyclic system (“bicyclic carbocyclyl”) or tricyclic system (“tricyclic carbocyclyl”)) and can be saturated or can contain one or more carbon-carbon double or triple bonds. “Carbocyclyl” also includes ring systems wherein the carbocyclyl ring, as defined above, is fused with one or more aryl or heteroaryl groups wherein the point of attachment is on the carbocyclyl ring, and in such instances, the number of carbons continue to designate the number of carbons in the carbocyclic ring system. Unless otherwise specified, each instance of a carbocyclyl group is independently unsubstituted (an “unsubstituted carbocyclyl”) or substituted (a “substituted carbocyclyl”) with one or more substituents. In certain embodiments, the carbocyclyl group is an unsubstituted C₃₋₁₀ carbocyclyl. In certain embodiments, the carbocyclyl group is a substituted C₃₋₁₀ carbocyclyl. In some embodiments, “carbocyclyl” is a monocyclic, saturated carbocyclyl group having from 3 to 10 ring carbon atoms (“C₃₋₁₀ cycloalkyl”). In some embodiments, a cycloalkyl group has 3 to 8 ring carbon atoms (“C₃₋₈ cycloalkyl”). In some embodiments, a cycloalkyl group has 3 to 6 ring carbon atoms (“C₃₋₆ cycloalkyl”). In some embodiments, a cycloalkyl group has 5 to 6 ring carbon atoms (“C₅₋₆ cycloalkyl”). In some embodiments, a cycloalkyl group has 5 to 10 ring carbon atoms (“C₅₋₁₀ cycloalkyl”). Examples of C₅₋₆ cycloalkyl groups include cyclopentyl (C₅) and cyclohexyl (C₅). Examples of C₃₋₆ cycloalkyl groups include the aforementioned C₅₋₆ cycloalkyl groups as well as cyclopropyl (C₃) and cyclobutyl (C₄). Examples of C₃₋₈ cycloalkyl groups include the aforementioned C₃₋₆ cycloalkyl groups as well as cycloheptyl (C₇) and cyclooctyl (C₈). Unless otherwise specified, each instance of a cycloalkyl group is independently unsubstituted (an “unsubstituted cycloalkyl”) or substituted (a “substituted cycloalkyl”) with one or more substituents. In certain embodiments, the cycloalkyl group is an unsubstituted C₃₋₁₀ cycloalkyl. In certain embodiments, the cycloalkyl group is a substituted C₃₋₁₀ cycloalkyl.

As used herein, “heterocyclyl” refers to a radical of a 3- to 14-membered non-aromatic ring system having ring carbon atoms and 1 to 4 ring heteroatoms, wherein each heteroatom is independently selected from nitrogen, oxygen, and sulfur (“3-14 membered heterocyclyl”). In heterocyclyl groups that contain one or more nitrogen atoms, the point of attachment can be a carbon or nitrogen atom, as valency permits. A heterocyclyl group can either be monocyclic (“monocyclic heterocyclyl”) or polycyclic (e.g., a fused, bridged or spiro ring system such as a bicyclic system (“bicyclic heterocyclyl”) or tricyclic system (“tricyclic heterocyclyl”)), and can be saturated or can contain one or more carbon-carbon double or triple bonds. Heterocyclyl polycyclic ring systems can include one or more heteroatoms in one or both rings. “Heterocyclyl” also includes ring systems wherein the heterocyclyl ring, as defined above, is fused with one or more carbocyclyl groups wherein the point of attachment is either on the carbocyclyl or heterocyclyl ring, or ring systems wherein the heterocyclyl ring, as defined above, is fused with one or more aryl or heteroaryl groups, wherein the point of attachment is on the heterocyclyl ring, and in such instances, the number of ring members continue to designate the number of ring members in the heterocyclyl ring system. Unless otherwise specified, each instance of heterocyclyl is independently unsubstituted (an “unsubstituted heterocyclyl”) or substituted (a “substituted heterocyclyl”) with one or more substituents. In certain embodiments, the heterocyclyl group is an unsubstituted 3-14 membered heterocyclyl. In certain embodiments, the heterocyclyl group is a substituted 3-14 membered heterocyclyl.

In some embodiments, a heterocyclyl group is a 5-10 membered non-aromatic ring system having ring carbon atoms and 1-4 ring heteroatoms, wherein each heteroatom is independently selected from nitrogen, oxygen, and sulfur (“5-10 membered heterocyclyl”). In some embodiments, a heterocyclyl group is a 5-8 membered non-aromatic ring system having ring carbon atoms and 1-4 ring heteroatoms, wherein each heteroatom is independently selected from nitrogen, oxygen, and sulfur (“5-8 membered heterocyclyl”). In some embodiments, a heterocyclyl group is a 5-6 membered non-aromatic ring system having ring carbon atoms and 1-4 ring heteroatoms, wherein each heteroatom is independently selected from nitrogen, oxygen, and sulfur (“5-6 membered heterocyclyl”). In some embodiments, the 5-6 membered heterocyclyl has 1-3 ring heteroatoms selected from nitrogen, oxygen, and sulfur. In some embodiments, the 5-6 membered heterocyclyl has 1-2 ring heteroatoms selected from nitrogen, oxygen, and sulfur. In some embodiments, the 5-6 membered heterocyclyl has 1 ring heteroatom selected from nitrogen, oxygen, and sulfur.

Exemplary 3-membered heterocyclyl groups containing 1 heteroatom include, without limitation, azirdinyl, oxiranyl, thiorenyl. Exemplary 4-membered heterocyclyl groups containing 1 heteroatom include, without limitation, azetidinyl, oxetanyl and thietanyl. Exemplary 5-membered heterocyclyl groups containing 1 heteroatom include, without limitation, tetrahydrofuranyl, dihydrofuranyl, tetrahydrothiophenyl, dihydrothiophenyl, pyrrolidinyl, dihydropyrrolyl and pyrrolyl-2,5-dione. Exemplary 5-membered heterocyclyl groups containing 2 heteroatoms include, without limitation, dioxolanyl, oxathiolanyl and dithiolanyl. Exemplary 5-membered heterocyclyl groups containing 3 heteroatoms include, without limitation, triazolinyl, oxadiazolinyl, and thiadiazolinyl. Exemplary 6-membered heterocyclyl groups containing 1 heteroatom include, without limitation, piperidinyl, tetrahydropyranyl, dihydropyridinyl, and thianyl. Exemplary 6-membered heterocyclyl groups containing 2 heteroatoms include, without limitation, piperazinyl, morpholinyl, dithianyl, dioxanyl. Exemplary 6-membered heterocyclyl groups containing 2 heteroatoms include, without limitation, triazinanyl. Exemplary 7-membered heterocyclyl groups containing 1 heteroatom include, without limitation, azepanyl, oxepanyl and thiepanyl. Exemplary 8-membered heterocyclyl groups containing 1 heteroatom include, without limitation, azocanyl, oxecanyl and thiocanyl. Exemplary bicyclic heterocyclyl groups include, without limitation, indolinyl, isoindolinyl, dihydrobenzofuranyl, dihydrobenzothienyl, tetrahydrobenzothienyl, tetrahydrobenzofuranyl, tetrahydroindolyl, tetrahydroquinolinyl, tetrahydroisoquinolinyl, decahydroquinolinyl, decahydroisoquinolinyl, octahydrochromenyl, octahydroisochromenyl, decahydronaphthyridinyl, decahydro-1,8-naphthyridinyl, octahydropyrrolo[3,2-b]pyrrole, indolinyl, phthalimidyl, naphthalimidyl, chromanyl, chromenyl, 1H-benzo[e][1,4]diazepinyl, 1,4,5,7-tetrahydropyrano[3,4-b]pyrrolyl, 5,6-dihydro-4H-furo[3,2-b]pyrrolyl, 6,7-dihydro-5H-furo[3,2-b]pyranyl, 5,7-dihydro-4H-thieno[2,3-c]pyranyl, 2,3-dihydro-1H-pyrrolo[2,3-b]pyridinyl, 2,3-dihydrofuro[2,3-b]pyridinyl, 4,5,6,7-tetrahydro-1H-pyrrolo-[2,3-b]pyridinyl, 4,5,6,7-tetrahydrofuro[3,2-c]pyridinyl, 4,5,6,7-tetrahydrothieno[3,2-b]pyridinyl, 1,2,3,4-tetrahydro-1,6-naphthyridinyl, and the like.

As used herein, “aryl” refers to a radical of a monocyclic or polycyclic (e.g., bicyclic or tricyclic) 4n+2 aromatic ring system (e.g., having 6, 10, or 14π electrons shared in a cyclic array) having 6-14 ring carbon atoms and zero heteroatoms provided in the aromatic ring system (“C₆₋₁₄ aryl”). In some embodiments, an aryl group has 6 ring carbon atoms (“C₆ aryl”; e.g., phenyl). In some embodiments, an aryl group has 10 ring carbon atoms (“C₁₀ aryl”; e.g., naphthyl such as 1-naphthyl and 2-naphthyl). In some embodiments, an aryl group has 14 ring carbon atoms (“C₁₄ aryl”; e.g., anthracyl). “Aryl” also includes ring systems wherein the aryl ring, as defined above, is fused with one or more carbocyclyl or heterocyclyl groups wherein the radical or point of attachment is on the aryl ring, and in such instances, the number of carbon atoms continue to designate the number of carbon atoms in the aryl ring system. Unless otherwise specified, each instance of an aryl group is independently unsubstituted (an “unsubstituted aryl”) or substituted (a “substituted aryl”) with one or more substituents. In certain embodiments, the aryl group is an unsubstituted C₆₋₁₄ aryl. In certain embodiments, the aryl group is a substituted C₆₋₁₄ aryl.

“Aralkyl” is a subset of “alkyl” and refers to an alkyl group, as defined herein, substituted by an aryl group, as defined herein, wherein the point of attachment is on the alkyl moiety.

As used herein, “heteroaryl” refers to a radical of a 5-14 membered monocyclic or polycyclic (e.g., bicyclic or tricyclic) 4n+2 aromatic ring system (e.g., having 6, 10, or 14π electrons shared in a cyclic array) having ring carbon atoms and 1-4 ring heteroatoms provided in the aromatic ring system, wherein each heteroatom is independently selected from nitrogen, oxygen and sulfur (“5-14 membered heteroaryl”). In heteroaryl groups that contain one or more nitrogen atoms, the point of attachment can be a carbon or nitrogen atom, as valency permits. Heteroaryl polycyclic ring systems can include one or more heteroatoms in one or both rings. “Heteroaryl” includes ring systems wherein the heteroaryl ring, as defined above, is fused with one or more carbocyclyl or heterocyclyl groups wherein the point of attachment is on the heteroaryl ring, and in such instances, the number of ring members continue to designate the number of ring members in the heteroaryl ring system. “Heteroaryl” also includes ring systems wherein the heteroaryl ring, as defined above, is fused with one or more aryl groups wherein the point of attachment is either on the aryl or heteroaryl ring, and in such instances, the number of ring members designates the number of ring members in the fused polycyclic (aryl/heteroaryl) ring system. Polycyclic heteroaryl groups wherein one ring does not contain a heteroatom (e.g., indolyl, quinolinyl, carbazolyl, and the like) the point of attachment can be on either ring, i.e., either the ring bearing a heteroatom (e.g., 2-indolyl) or the ring that does not contain a heteroatom (e.g., 5-indolyl). In some embodiments, a heteroaryl group is a 5-10 membered aromatic ring system having ring carbon atoms and 1-4 ring heteroatoms provided in the aromatic ring system, wherein each heteroatom is independently selected from nitrogen, oxygen, and sulfur (“5-10 membered heteroaryl”). In some embodiments, a heteroaryl group is a 5-8 membered aromatic ring system having ring carbon atoms and 1-4 ring heteroatoms provided in the aromatic ring system, wherein each heteroatom is independently selected from nitrogen, oxygen, and sulfur (“5-8 membered heteroaryl”). In some embodiments, a heteroaryl group is a 5-6 membered aromatic ring system having ring carbon atoms and 1-4 ring heteroatoms provided in the aromatic ring system, wherein each heteroatom is independently selected from nitrogen, oxygen, and sulfur (“5-6 membered heteroaryl”). In some embodiments, the 5-6 membered heteroaryl has 1-3 ring heteroatoms selected from nitrogen, oxygen, and sulfur. In some embodiments, the 5-6 membered heteroaryl has 1-2 ring heteroatoms selected from nitrogen, oxygen, and sulfur. In some embodiments, the 5-6 membered heteroaryl has 1 ring heteroatom selected from nitrogen, oxygen, and sulfur. Unless otherwise specified, each instance of a heteroaryl group is independently unsubstituted (an “unsubstituted heteroaryl”) or substituted (a “substituted heteroaryl”) with one or more substituents. In certain embodiments, the heteroaryl group is an unsubstituted 5-14 membered heteroaryl. In certain embodiments, the heteroaryl group is a substituted 5-14 membered heteroaryl.

Exemplary 5-membered heteroaryl groups containing 1 heteroatom include, without limitation, pyrrolyl, furanyl and thiophenyl. Exemplary 5-membered heteroaryl groups containing 2 heteroatoms include, without limitation, imidazolyl, pyrazolyl, oxazolyl, isoxazolyl, thiazolyl, and isothiazolyl. Exemplary 5-membered heteroaryl groups containing 3 heteroatoms include, without limitation, triazolyl, oxadiazolyl, and thiadiazolyl. Exemplary 5-membered heteroaryl groups containing 4 heteroatoms include, without limitation, tetrazolyl. Exemplary 6-membered heteroaryl groups containing 1 heteroatom include, without limitation, pyridinyl. Exemplary 6-membered heteroaryl groups containing 2 heteroatoms include, without limitation, pyridazinyl, pyrimidinyl, and pyrazinyl. Exemplary 6-membered heteroaryl groups containing 3 or 4 heteroatoms include, without limitation, triazinyl and tetrazinyl, respectively. Exemplary 7-membered heteroaryl groups containing 1 heteroatom include, without limitation, azepinyl, oxepinyl, and thiepinyl. Exemplary 5,6-bicyclic heteroaryl groups include, without limitation, indolyl, isoindolyl, indazolyl, benzotriazolyl, benzothiophenyl, isobenzothiophenyl, benzofuranyl, benzoisofuranyl, benzimidazolyl, benzoxazolyl, benzisoxazolyl, benzoxadiazolyl, benzthiazolyl, benzisothiazolyl, benzthiadiazolyl, indolizinyl, and purinyl. Exemplary 6,6-bicyclic heteroaryl groups include, without limitation, naphthyridinyl, pteridinyl, quinolinyl, isoquinolinyl, cinnolinyl, quinoxalinyl, phthalazinyl, and quinazolinyl. Exemplary tricyclic heteroaryl groups include, without limitation, phenanthridinyl, dibenzofuranyl, carbazolyl, acridinyl, phenothiazinyl, phenoxazinyl and phenazinyl.

“Heteroaralkyl” is a subset of “alkyl” and refers to an alkyl group, as defined herein, substituted by a heteroaryl group, as defined herein, wherein the point of attachment is on the alkyl moiety.

As used herein, the term “partially unsaturated” refers to a ring moiety that includes at least one double or triple bond. The term “partially unsaturated” is intended to encompass rings having multiple sites of unsaturation, but is not intended to include aromatic groups (e.g., aryl or heteroaryl moieties) as herein defined.

As used herein, a “direct bond” refers to the direct attachment of a group via a single bond.

Alkyl, alkenyl, alkynyl, carbocyclyl, heterocyclyl, aryl, and heteroaryl groups, as defined herein, are optionally substituted (e.g., “substituted” or “unsubstituted” alkyl, “substituted” or “unsubstituted” alkenyl, “substituted” or “unsubstituted” alkynyl, “substituted” or “unsubstituted” carbocyclyl, “substituted” or “unsubstituted” heterocyclyl, “substituted” or “unsubstituted” aryl or “substituted” or “unsubstituted” heteroaryl group). In general, the term “substituted”, whether preceded by the term “optionally” or not, means that at least one hydrogen present on a group (e.g., a carbon or nitrogen atom) is replaced with a permissible substituent, e.g., a substituent which upon substitution results in a stable compound, e.g., a compound which does not spontaneously undergo transformation such as by rearrangement, cyclization, elimination, or other reaction. Unless otherwise indicated, a “substituted” group has a substituent at one or more substitutable positions of the group, and when more than one position in any given structure is substituted, the substituent is either the same or different at each position. The term “substituted” is contemplated to include substitution with all permissible substituents of organic compounds, any of the substituents described herein that results in the formation of a stable compound. The present invention contemplates any and all such combinations in order to arrive at a stable compound. For purposes of this invention, heteroatoms such as nitrogen may have hydrogen substituents and/or any suitable substituent as described herein which satisfy the valencies of the heteroatoms and results in the formation of a stable moiety.

Exemplary carbon atom substituents include, but are not limited to, halogen, —CN, —NO₂, —N₃, —SO₂H, —SO₃H, —OH, —OR^{aa}, —ON(R^{bb})₂, —N(R^{bb})₂, —N(R^{bb})₃ ⁺X⁻, —N(OR^{cc})R^{bb}, —SH, —SR^{aa}, —SSR^{cc}, —C(═O)R^{aa}, —CO₂H, —CHO, —C(OR^{cc})₂, —CO₂R^{aa}, —OC(═O)R^{aa}, —OCO₂R^{aa}, —C(═O)N(R^{bb})₂, —OC(═O)N(R^{bb})₂, —NR^{bb}C(═O)R^{aa}, —NR^{bb}CO₂R^{aa}, —NR^{bb}C(═O)N(R^{bb})₂, —C(═NR^{bb})R^{aa}, —C(═NR^{bb})OR^{aa}, —OC(═NR^{bb})R^{aa}, —OC(═NR^{bb})OR^{aa}, —C(═NR^{bb})N(R^{bb})₂, —OC(═NR^{bb})N(R^{bb})₂, —NR^{bb}C(═NR^{bb})N(R^{bb})₂, —C(═O)NR^{bb}SO₂R^{aa}, —NR^{bb}SO₂R^{aa}, —SO₂N(R^{bb})₂, —SO₂R^{aa}, —SO₂OR^{aa}, —OSO₂R^{aa}, —S(═O)R^{aa}, —OS(═O)R^{aa}, —Si(R^{aa})₃, —OSi(R^{aa})₃—C(═S)N(R^{bb})₂, —C(═O)SR^{aa}, —C(═S)SR^{aa}, —SC(═S)SR^{aa}, —SC(═O)SR^{aa}, —OC(═O)SR^{aa}, —SC(═O)OR^{aa}, —SC(═O)R^{aa}, —P(═O)₂R^{aa}, —OP(═O)₂R^{aa}, —P(═O)(R^{aa})₂, —OP(═O)(R^{aa})₂, —OP(═O)(OR^{cc})₂, —P(═O)₂N(R^{bb})₂, —OP(═O)₂N(R^{bb})₂, —P(═O)(NR^{bb})₂, —OP(═O)(NR^{bb})₂, —NR^{bb}P(═O)(OR^{cc})₂, —NR^{bb}P(═O)(NR^{bb})₂, —P(R^{cc})₂, —P(R^{cc})₃, —OP(R^{cc})₂, —OP(R^{cc})₃, —B(R^{aa})₂, —B(OR^{cc})₂, —BR^{aa}(OR^{cc}), C₁₋₁₀ alkyl, C₁₋₁₀ perhaloalkyl, C₂₋₁₀ alkenyl, C₂₋₁₀ alkynyl, C₃₋₁₄ carbocyclyl, ₃₋₁₄ membered heterocyclyl, C₆₋₁₄ aryl, and 5-14 membered heteroaryl, wherein each alkyl, alkenyl, alkynyl, carbocyclyl, heterocyclyl, aryl, and heteroaryl is independently substituted with 0, 1, 2, 3, 4, or 5 R^{dd} groups; or two geminal hydrogens on a carbon atom are replaced with the group ═O, ═S, ═NN(R^{bb})₂, ═NNR^{bb}C(═O)R^{aa}, ═NNR^{bb}C(═O)OR^{aa}, —NNR^{bb}S(═O)₂R^{aa}, ═NR^{bb}, or ═NOR^{cc}; each instance of R^{aa} is, independently, selected from C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{aa} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups;
each instance of R^{bb} is, independently, selected from hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{cc})OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C(═O)SR^{cc}, —C(═S)SR^{cc}, —P(═O)₂R^{aa}, —P(═O)(R^{aa})₂, —P(═O)₂N(R^{cc})₂, —P(═O)(NR^{cc})₂, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{bb} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{cc} is, independently, selected from hydrogen, C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two R^{cc} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups; each instance of R^{dd} is, independently, selected from halogen, —CN, —NO₂, —N₃, —SO₂H, —SO₃H, —OH, —OR^{ee}, —ON(R^{ff})₂, —N(R^{ff})₂, —N(R^{ff})₃ ⁺X⁻, —N(OR^{ee})R^{ff}, —SH, —SR^{ee}, —SSR^{ee}, —C(═O)R^{ee}, —CO₂H, —CO₂R^{ee}, —OC(═O) R^{ee}, —OCO₂R^{ee}, —C(═O)N(R^{ff})₂, —OC(═O)N(R^{ff})₂, —NR^{ff}C(═O)R^{ee}, —NR^{ff}CO₂R^{ee}, —NR^{ff}C(═O)N(R^{ff})₂, —C(═NR^{ff})OR^{ee}, —OC(═NR^{ff})R^{ee}, —OC(═NR^{ff})OR^{ee}, —C(═NR^{ff})N(R^{ff})₂, —OC(═NR^{ff})N(R^{ff})₂, —NR^{ff}C(═NR^{ff})N(R^{ff})₂, —NR^{ff}SO₂R^{ee}, —SO₂N(R^{ff})₂, —SO₂R^{ee}, —SO₂OR^{ee}, —OSO₂R^{ee}, —S(═O)R^{ee}, —Si(R^{ee})₃, —OSi(R^{ee})₃, —C(═S)N(R^{ff})₂, —C(═O)SR^{ee}, —C(═S)SR^{ee}, —SC(═S)SR^{ee}, —P(═O)₂R^{ee}, —P(═O)(R^{ee})₂, —OP(═O)(R^{ee})₂, —OP(═O)(OR^{ee})₂, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl, 5-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups, or two geminal R^{dd} substituents can be joined to form ═O or ═S; each instance of R^{ee} is, independently, selected from C₁₋₆ alkyl, C₁₋₆ perhaloalkyl, C₂₋₆ alkenyl, C₂₋₆ alkynyl, C₃₋₁₀ carbocyclyl, C₆₋₁₀ aryl, 3-10 membered heterocyclyl, and 3-10 membered heteroaryl, wherein each alkyl, alkenyl, alkynyl, carbocyclyl, heterocyclyl, aryl, and heteroaryl is independently substituted with 0, 1, 2, 3, 4, or 5 R^{gg} groups; each instance of R^{ff} is, independently, selected from hydrogen, C₁₋₆ alkyl, C₁₋₆ perhaloalkyl, C₂₋₆ alkenyl, C₂₋₆ alkynyl, C₃₋₁₀ carbocyclyl, 3-10 membered heterocyclyl, C₆₋₁₀ aryl and 5-10 membered heteroaryl, or two R^{ff} groups are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{gg} groups; and each instance of R^{gg} is, independently, halogen, —CN, —NO₂, —N₃, —SO₂H, —SO₃H, —OH, —OC₁₋₆ alkyl, —ON(C₁₋₆ alkyl)₂, —N(C₁₋₆ alkyl)₂, —N(C₁₋₆ alkyl)₃ ⁺X⁻, —NH(C₁₋₆ alkyl)₂ ⁺X⁻, —NH₂(C₁₋₆ alkyl)⁺X⁻, —NH₃ ⁺X⁻, —N(OC₁₋₆ alkyl)(C₁₋₆ alkyl), —N(OH)(C₁₋₆ alkyl), —NH(OH), —SH, —SC₁₋₆ alkyl, —SS(C₁₋₆ alkyl), —C(═O)(C₁₋₆ alkyl), —CO₂H, —CO₂(C₁₋₆ alkyl), —OC(—O)(C₁₋₆ alkyl), —OCO₂(C₁₋₆ alkyl), —C(═O)NH₂, —C(═O)N(C₁₋₆ alkyl)₂, —OC(═O)NH(C₁₋₆ alkyl), —NHC(═O)(C₁₋₆ alkyl), —N(C₁₋₆ alkyl)C(═O)(C₁₋₆ alkyl), —NHCO₂(C₁₋₆ alkyl), —NHC(═O)N(C₁₋₆ alkyl)₂, —NHC(═O)NH(C₁₋₆ alkyl), —NHC(═O)NH₂, —C(═NH)O(C₁₋₆ alkyl), —OC(═NH)(C₁₋₆ alkyl), —OC(═NH)OC₁₋₆ alkyl, —C(═NH)N(C₁₋₆ alkyl)₂, —C(═NH)NH(C₁₋₆ alkyl), —C(═NH)NH₂, —OC(═NH)N(C₁₋₆ alkyl)₂, —OC(NH)NH(C₁₋₆ alkyl), —OC(NH)NH₂, —NHC(NH)N(C₁₋₆ alkyl)₂, —NHC(═NH)NH₂, —NHSO₂(C₁₋₆ alkyl), —SO₂N(C₁₋₆ alkyl)₂, —SO₂NH(C₁₋₆ alkyl), —SO₂NH₂, —SO₂C₁₋₆ alkyl, —SO₂OC₁₋₆ alkyl, —OSO₂C₁₋₆ alkyl, —SOC₁₋₆ alkyl, —Si(C₁₋₆ alkyl)₃, —OSi(C₁₋₆ alkyl)₃—C(═S)N(C₁₋₆ alkyl)₂, C(═S)NH(C₁₋₆ alkyl), C(═S)NH₂, —C(═O)S(C₁₋₆ alkyl), —C(═S)SC₁₋₆ alkyl, —SC(═S)SC₁₋₆ alkyl, —P(═O)₂(C₁₋₆ alkyl), —P(═O)(C₁₋₆ alkyl)₂, —OP(═O)(C₁₋₆ alkyl)₂, —OP(═O)(OC₁₋₆ alkyl)₂, C₁₋₆ alkyl, C₁₋₆ perhaloalkyl, C₂₋₆ alkenyl, C₂₋₆ alkenyl, C₃₋₁₀ carbocyclyl, C₆₋₁₀ aryl, 3-10 membered heterocyclyl, 5-10 membered heteroaryl; or two geminal R^{gg} substituents can be joined to form ═O or ═S;
wherein X⁻ is a counterion.

As used herein, the term “hydroxyl” or “hydroxy” refers to the group —OH. The term “substituted hydroxyl” or “substituted hydroxyl,” by extension, refers to a hydroxyl group wherein the oxygen atom directly attached to the parent molecule is substituted with a group other than hydrogen, and includes groups selected from —OR^{aa}, —ON(R^{bb})₂, —OC(═O)SR^{aa}, —OC(═O)R^{aa}, —OCO₂R^{aa}, —OC(═O)N(R^{bb})₂, —OC(═NR^{bb})R^{aa}, —OC(═NR^{bb})OR^{aa}, —OC(═NR^{bb})N(R^{bb})₂, —OS(═O)R^{aa}, —OSO₂R^{aa}, —OSi(R^{aa})₃, —OP(R^{cc})₂, —OP(R^{cc})₃, —OP(═O)₂R^{aa}, —OP(═O)(R^{aa})₂, —OP(═O)(OR^{cc})₂, —OP(═O)₂N(R^{bb})₂, and —OP(═O)(NR^{bb})₂, wherein R^{aa}, R^{bb}, and R^{cc} are as defined herein.

As used herein, the term “thiol” or “thio” refers to the group —SH. The term “substituted thiol” or “substituted thio,” by extension, refers to a thiol group wherein the sulfur atom directly attached to the parent molecule is substituted with a group other than hydrogen, and includes groups selected from —SR^{aa}, —S═SR^{cc}, —SC(═S)SR^{aa}, —SC(═O)SR^{aa}, —SC(═O)OR^{aa}, and —SC(═O)R^{aa}, wherein R^{aa} and R^{cc} are as defined herein.

As used herein, the term, “amino” refers to the group —NH₂. The term “substituted amino,” by extension, refers to a monosubstituted amino or a disubstituted amino, or a trisubstituted amino, as defined herein. In certain embodiments, the “substituted amino” is a monosubstituted amino or a disubstituted amino group.

As used herein, the term “monosubstituted amino” refers to an amino group wherein the nitrogen atom directly attached to the parent molecule is substituted with one hydrogen and one group other than hydrogen, and includes groups selected from —NH(R^{bb}), —NHC(═O)R^{aa}, —NHCO₂R^{aa}, —NHC(═O)N(R^{bb})₂, —NHC(═NR^{bb})N(R^{bb})₂, —NHSO₂R^{aa}, —NHP(═O)(OR^{cc})₂, and —NHP(═O)(NR^{bb})₂, wherein R^{aa}, R^{bb} and R^{cc} are as defined herein, and wherein R^{bb} of the group —NH(R^{bb}) is not hydrogen.

As used herein, the term “disubstituted amino” refers to an amino group wherein the nitrogen atom directly attached to the parent molecule is substituted with two groups other than hydrogen, and includes groups selected from —N(R^{bb})₂, —NR^{bb}C(═O)R^{aa}, —NR^{bb}CO₂R^{aa}, —NR^{bb}C(═O)N(R^{bb})₂, —NR^{bb}C(═NR^{bb})N(R^{bb})₂, —NR^{bb}SO₂R^{aa}, —NR^{bb}P(═O)(OR^{cc})₂, and —NR^{bb}P(═O)(NR^{bb})₂, wherein R^{aa}, R^{bb}, and R^{cc} are as defined herein, with the proviso that the nitrogen atom directly attached to the parent molecule is not substituted with hydrogen.

As used herein, the term “trisubstituted amino” refers to an amino group wherein the nitrogen atom directly attached to the parent molecule is substituted with three groups, and includes groups selected from —N(R^{bb})₃ and —N(R^{bb})₃ ⁺X⁻, wherein R^{bb} and X are as defined herein.

As used herein, the term “sulfonyl” refers to a group selected from —SO₂N(R^{bb})₂, —SO₂R^{aa}, and —SO₂OR^{aa}, wherein R^{aa} and R^{bb} are as defined herein.

As used herein, the term “sulfinyl” refers to the group —S(═O)R^{aa}, wherein R^{aa} is as defined herein.

As used herein, the term “carbonyl” refers a group wherein the carbon directly attached to the parent molecule is sp² hybridized, and is substituted with an oxygen, nitrogen or sulfur atom, e.g., a group selected from ketones (—C(═O)R^{aa}), carboxylic acids (—CO₂H), aldehydes (—CHO), esters (—CO₂R^{aa}, —C(═O)SR^{aa}, —C(═S)SR^{aa}), amides (—C(═O)N(R^{bb})₂, —C(═O)NR^{bb}SO₂R^{aa}, —C(═S)N(R^{bb})₂), and imines (—C(═NR^{bb})R^{aa}, —C(═NR^{bb})OR^{aa}), —C(═NR^{bb})N(R^{bb})₂), wherein R^{aa} and R^{bb} are as defined herein.

As used herein, the term “silyl” refers to the group —Si(R^{aa})₃, wherein R^{aa} is as defined herein.

As used herein, a “counterion” is a negatively charged group associated with a positively charged quarternary amine in order to maintain electronic neutrality. Exemplary counterions include halide ions (e.g., F⁻, Cl⁻, Br⁻, I⁻), NO₃⁻, ClO₄⁻, OH⁻, H₂PO₄⁻, HSO₄⁻, sulfonate ions (e.g., methansulfonate, trifluoromethanesulfonate, p-toluenesulfonate, benzenesulfonate, 10-camphor sulfonate, naphthalene-2-sulfonate, naphthalene-1-sulfonic acid-5-sulfonate, ethan-1-sulfonic acid-2-sulfonate, and the like), and carboxylate ions (e.g., acetate, ethanoate, propanoate, benzoate, glycerate, lactate, tartrate, glycolate, and the like).

Nitrogen atoms can be substituted or unsubstituted as valency permits, and include primary, secondary, tertiary, and quarternary nitrogen atoms. Exemplary nitrogen atom substituents include, but are not limited to, hydrogen, —OH, —OR^{aa}, —N(R^{cc})₂, —CN, —C(═O)R^{aa}, —C(═O)N(R^{cc})₂, —CO₂R^{aa}, —SO₂R^{aa}, —C(═NR^{bb})R^{aa}, —C(═NR^{cc}) OR^{aa}, —C(═NR^{cc})N(R^{cc})₂, —SO₂N(R^{cc})₂, —SO₂R^{cc}, —SO₂OR^{cc}, —SOR^{aa}, —C(═S)N(R^{cc})₂, —C₁₋₁₀ alkyl, C₁₋₁₀ perhaloalkyl, C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl, or two Rec groups attached to an N atom are joined to form a 3-14 membered heterocyclyl or 5-14 membered heteroaryl ring, wherein each alkyl, alkenyl, alkynyl, carbocyclyl, heterocyclyl, aryl, and heteroaryl is independently substituted with 0, 1, 2, 3, 4, or 5 R^{dd} groups, and wherein R^{aa}, R^{bb}, R^{cc} and R^{dd} are as defined above.

In certain embodiments, the substituent present on the nitrogen atom is an amino protecting group Amino protecting groups include, but are not limited to, —OH, —OR^{aa}, —N(R^{cc})₂, —C(═O)R^{aa}, —C(═O)N(R^{cc})₂, —CO₂R^{aa}, —SO₂R^{aa}, —C(═NR^{cc}) R^{aa}, —C(═NR^{cc})OR^{aa}, —C(═NR^{cc})N(R^{cc})₂, —SO₂N(R^{cc})₂, —SO₂R^{cc}, SO₂OR^{cc}, —SOR^{aa}, —C(═S)N(R^{cc})₂, —C(═O)SR^{cc}, —C(═S)SR^{cc}, C₁₋₁₀ alkyl (e.g., aralkyl, heteroaralkyl), C₂₋₁₀ alkenyl, C₂₋₁₀ alkynyl, C₃₋₁₀ carbocyclyl, 3-14 membered heterocyclyl, C₆₋₁₄ aryl, and 5-14 membered heteroaryl groups, wherein each alkyl, alkenyl, alkynyl, carbocyclyl, heterocyclyl, aralkyl, aryl, and heteroaryl is independently substituted with 0, 1, 2, 3, 4, or 5 R^{dd} groups, and wherein R^{aa}, R^{bb}, R^{cc} and R^{dd} are as defined herein Amino protecting groups are well known in the art and include those described in detail in Protecting Groups in Organic Synthesis, T. W. Greene and P. G. M. Wuts, 3^{rd} edition, John Wiley & Sons, 1999, incorporated herein by reference.

These and other exemplary substituents are described in more detail in the Detailed Description, the Examples and in the claims. The invention is not intended to be limited in any manner by the above exemplary listing of substituents.

The compounds useful in the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. As used herein, the term “isomers” includes any and all geometric isomers and stereoisomers. For example, “isomers” include cis-and trans-isomers, E- and Z-isomers, R- and S-enantiomers, diastereomers, (D)-isomers, (L)-isomers, racemic mixtures thereof, and other mixtures thereof, as falling within the scope of the invention. For instance, an isomer/enantiomer may, in some embodiments, be provided substantially free of the corresponding enantiomer, and may also be referred to as “optically enriched.” “Optically enriched,” as used herein, means that the compound is made up of a significantly greater proportion of one enantiomer. In certain embodiments the compound of the present invention is made up of at least about 90% by weight of a particular enantiomer. In other embodiments the compound is made up of at least about 95%, 98%, or 99% by weight of a particular enantiomer. A desired enantiomer may be isolated from a racemic mixture by any method known to those skilled in the art, including chiral high pressure liquid chromatography (HPLC) or the formation and crystallization of chiral salts, or the enantiomer may be prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

As used herein, the term “tautomer” includes two or more interconvertable compounds resulting from at least one formal migration of a hydrogen atom and at least one change in valency (e.g., a single bond to a double bond, a triple bond to a single bond, or vice versa). The exact ratio of the tautomers depends on several factors, including temperature, solvent, and pH. Tautomerizations (i.e., the reaction providing a tautomeric pair) may catalyzed by acid or base. Exemplary tautomerizations include keto-to-enol; amide-to-imide; lactam-to-lactim; enamine-to-imine; and enamine-to-(a different) enamine tautomerizations.

The term “carbohydrate” refers to a sugar or polymer of sugars. The terms “saccharide,” “polysaccharide,” “carbohydrate,” and “oligosaccharide,” may be used interchangeably. Most carbohydrates are aldehydes or ketones with many hydroxyl groups, usually one on each carbon atom of the molecule. Carbohydrates generally have the molecular formula CₙH₂ₙOₙ. A carbohydrate may be a monosaccharide, a disaccharide, trisaccharide, oligosaccharide, or polysaccharide. The most basic carbohydrate is a monosaccharide, such as glucose, sucrose, galactose, mannose, ribose, arabinose, xylose, and fructose. Disaccharides are two joined monosaccharides. Exemplary disaccharides include sucrose, maltose, cellobiose, and lactose. Typically, an oligosaccharide includes between three and six monosaccharide units (e.g., raffinose, stachyose), and polysaccharides include six or more monosaccharide units. Exemplary polysaccharides include starch, glycogen, and cellulose. Carbohydrates may contain modified saccharide units such as 2′-deoxyribose wherein a hydroxyl group is removed, 2′-fluororibose wherein a hydroxyl group is replace with a fluorine, or N-acetylglucosamine, a nitrogen-containing form of glucose. (e.g., 2′-fluororibose, deoxyribose, and hexose). Carbohydrates may exist in many different forms, for example, conformers, cyclic forms, acyclic forms, stereoisomers, tautomers, anomers, and isomers.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts are well known in the art. For example, Berge et al., describe pharmaceutically acceptable salts in detail in J. Pharmaceutical Sciences, 1977, 66, 1-19, incorporated herein by reference. Pharmaceutically acceptable salts of the compounds useful in this invention include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Salts derived from appropriate bases include alkali metal, alkaline earth metal, ammonium and N⁺ (C₁₋₄alkyl)₄ salts. Representative alkali or alkaline earth metal salts include sodium, lithium, potassium, calcium, magnesium, and the like. Other pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate, and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moeity advantageously has not more than 6 carbon atoms. Examples of particular esters include formates, acetates, propionates, butyrates, acrylates and ethylsuccinates.

Furthermore, the term “pharmaceutically acceptable prodrugs” as used herein refers to those prodrugs of the compounds useful in the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds useful in the invention. The term “prodrug” refers to compounds that are rapidly transformed in vivo to yield the parent compound of the above formula, for example by hydrolysis in blood. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester or an ether which is cleaved in vivo to yield a compound of interest. Pro-drugs of a variety of compounds, and materials and methods for derivatizing the parent compounds to create the pro-drugs, are known and may be adapted to the present invention. The biological activity of pro-drugs and pro-drugs may also be altered by appending a functionality onto the compound, which may be catalyzed by an enzyme. Also, included are oxidation and reduction reactions, including enzyme-catalyzed oxidation and reduction reactions. A thorough discussion of prodrugs is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As used herein the term “inhibit” means to reduce the amount of kinase activity to a level or amount that is statistically significantly less than an initial level, which may be a baseline level of kinase activity.

As used herein, the terms “treatment,” “treat,” and “treating” refer to reversing, alleviating, delaying the onset of, or inhibiting the progress of a disease or disorder, or one or more symptoms thereof, as described herein. In some embodiments, treatment may be administered after one or more symptoms have developed. In other embodiments, treatment may be administered in the absence of symptoms. For example, treatment may be administered to a susceptible individual prior to the onset of symptoms (e.g., in light of a history of symptoms and/or in light of genetic or other susceptibility factors). Treatment may also be continued after symptoms have resolved, for example to prevent or delay their recurrence.

As used herein, unless otherwise specified, the terms “prevent,” “preventing” and “prevention” contemplate an action that occurs before a subject begins to suffer from the specified disease or disorder, which inhibits or reduces the severity of the disease or disorder.

The terms “administer,” “administering,” or “administration,” as used herein refers to implanting, absorbing, ingesting, injecting, or inhaling the compound.

The term “subject” refers to any animal. The subject may be at any stage of development. A “subject” to which administration is contemplated includes, but is not limited to, humans (i.e., a male or female of any age group, e.g., a pediatric subject (e.g, infant, child, adolescent) or adult subject (e.g., young adult, middle-aged adult or senior adult)) and/or other primates (e.g., cynomolgus monkeys, rhesus monkeys); mammals, including commercially relevant mammals such as cattle, pigs, horses, sheep, goats, cats, and/or dogs; and/or birds, including commercially relevant birds such as chickens, ducks, geese, and/or turkeys. In some embodiments, the subject is a rodent. In certain embodiments, the subject is an experimental animal such as a mouse, rat, dog, or non-human primate. In certain embodiments, the subject is a transgenic animal.

The term “proliferative disorder” as used herein refers to any disease associated with an undesired and/or abnormal proliferation of cells. The cells may be any type of cell found in the subject. The proliferation may be due to any cause (e.g., any genetic mutation, any signal).

A therapeutically effective amount of a compound comprises administering an amount necessary to achieve a desired result. The exact amount required will vary from subject to subject, depending on the species, age, general condition of the subject, the severity of the disease, the particular anticancer agent, its mode of administration, the desired outcome, and the like. In certain embodiments of the present invention, a “therapeutically effective amount” of a compound or pharmaceutical composition is that amount effective for inhibiting cell proliferation in a subject or a biological sample (e.g., in cells). In certain embodiments, cell proliferation is inhibited by about 10%, about 20%, about 30%, about 40%, about 50%, about 60%, about 70%, about 80%, about 90%, about 95%, or about 99%. In certain embodiments, the compound inhibits cell proliferation by at least about 25%, at least about 50%, at least about 75%, or at least about 90%. In certain embodiments of the present invention, a “therapeutically effective amount” refers to an amount of a compound or composition sufficient to inhibit cell proliferation, or refers to an amount of a compound or composition sufficient to reduce the tumor burden in a subject. In certain embodiments, the tumor burden is reduced by about 1%, about 5%, about 10%, about 20%, about 30%, about 40%, about 50%, about 60%, about 70%, about 80%, about 90%, about 95%, or about 99%. In certain embodiments, the tumor burden is reduced by at least about 25%, at least about 50%, at least about 75%, or at least about 90%. In certain embodiments of the present invention a “therapeutically effective amount” of the compound or pharmaceutical composition is that amount effective for reducing or inhibiting the growth of tumor cells and/or killing tumor cells.

As used herein, a “prophylactically effective amount” of a compound is an amount sufficient to prevent a disease or disorder, or one or more symptoms associated with the disease or disorder, or prevent its recurrence. A prophylactically effective amount of a compound means an amount of therapeutic agent, alone or in combination with other agents, which provides a prophylactic benefit in the prevention of the disease or disorder. The term “prophylactically effective amount” can encompass an amount that improves overall prophylaxis or enhances the prophylactic efficacy of another prophylactic agent.

### DETAILED DESCRIPTION OF THE PRESENT INVENTION

The present invention provides a pharmaceutical composition comprising a combination of (a) a compound of Formula (A) and (b) one or more antineoplastic agents, wherein:

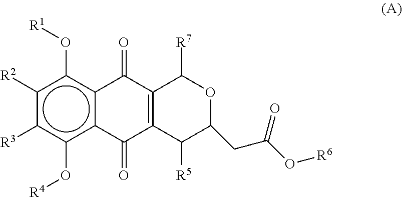

or a pharmaceutically acceptable salt thereof,
wherein:
each instance of R¹ and R⁴ is independently selected from the group consisting of hydrogen, carbonyl, silyl, optionally substituted alkyl, optionally substituted alkenyl, optionally substituted alkynyl, optionally substituted carbocyclyl, optionally substituted heterocyclyl, optionally substituted aryl, optionally substituted heteroaryl;
each instance of R² and R³ is independently selected from the group consisting of hydrogen, halogen, —OH, substituted hydroxyl, —SH, substituted thiol, —NH₂, substituted amino, —CN, —NO₂, carbonyl, silyl, sulfinyl, sulfonyl, optionally substituted alkyl, optionally substituted alkenyl, optionally substituted alkynyl, optionally substituted carbocyclyl, optionally substituted heterocyclyl, optionally substituted aryl, and optionally substituted heteroaryl; or R² and R³ are joined to form an optionally substituted carbocyclyl, optionally substituted heterocyclyl, optionally substituted aryl, or optionally substituted heteroaryl group;
R⁵ is hydrogen and R⁶ is selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl; or R⁵ and R⁶ are joined to form a direct bond;
R⁷ is hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl;
In certain embodiments, R¹ is hydrogen. In certain embodiments, R¹ is carbonyl, silyl, optionally substituted carbocyclyl, optionally substituted heterocyclyl, optionally substituted aryl, or optionally substituted heteroaryl.
In certain embodiments, R⁴ is hydrogen. In certain embodiments, R⁴ is optionally substituted carbocyclyl, optionally substituted heterocyclyl, optionally substituted aryl, or optionally substituted heteroaryl.
In certain embodiments, both R¹ and R⁴ are hydrogen, e.g., to provide a compound of the Formula (A-1):

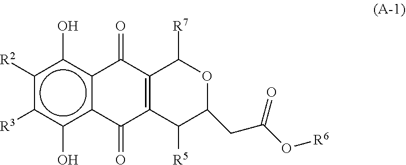

or a pharmaceutically acceptable salt thereof,
wherein R³, R⁴, R⁵, R⁶, and R⁷ are as defined herein.

In certain embodiments, R² is hydrogen. In certain embodiments, R² is optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, R² is optionally substituted carbocyclyl, optionally substituted heterocyclyl, optionally substituted aryl, or optionally substituted heteroaryl. In certain embodiments, R² is optionally substituted heterocyclyl (e.g., optionally substituted tetrahydropyranyl).

In certain embodiments, R² is an optionally substituted tetrahydropyranyl group of the formula (i):

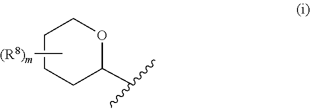

wherein:
each instance of R⁸ is independently selected from optionally substituted alkyl, optionally substituted alkenyl, optionally substituted alkynyl, optionally substituted carbocyclyl, optionally substituted heterocyclyl, optionally substituted aryl, optionally substituted heteroaryl, —OH, substituted hydroxyl, —SH, substituted thiol, —NH₂, substituted amino, —CN, —NO₂, —N₂, carbonyl, silyl, sulfonyl, and sulfinyl; and m is 0 or an integer of between 1 and 5, inclusive.

In certain embodiments, each instance of R⁸ is independently selected from optionally substituted alkyl, —OH, substituted hydroxyl, —SH, substituted thiol, —NH₂, substituted amino, carbonyl, and silyl. In certain embodiments, each instance of R⁷ is independently selected from optionally substituted alkyl, —OH, substituted hydroxyl, —NH₂, and substituted amino. In certain embodiments, each instance of R⁸ is independently selected from optionally substituted alkyl, —OH, and substituted amino (e.g., disubstituted amino) In certain embodiments, each instance of R⁸ is independently selected from —CH₃, —OH, and —N(CH₃)₂.

In certain embodiments, m is an integer of between 1 and 4, inclusive. In certain embodiments, m is an integer of between 1 and 3, inclusive. In certain embodiments, m is an integer of between 1 and 2, inclusive. In certain embodiments, m is 3.

In certain embodiments, each instance of R⁸ is independently selected from —CH₃, —OH, and —N(CH₃)₂, and m is an integer of between 1 and 3, inclusive. For example, in this instance, in certain embodiments, R² is a substituted tetrahydropyranyl group of the formula (ii):

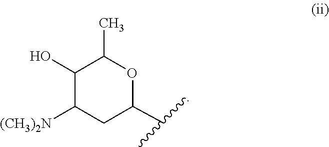

In certain embodiments, the substituted tetrahydropyranyl group of the formula (ii) is of the formula (iii):

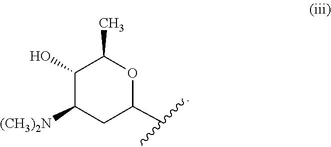

Alternatively, in certain embodiments, R² is an optionally substituted aryl or optionally substituted heteroaryl. In certain embodiments, R² is an optionally substituted aryl (e.g., a benzoisochromanequinone).

In certain embodiments R² is substituted benzoisochromanequinone of the formula (iv):

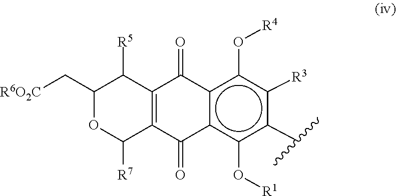

wherein R¹, R³, R⁴, R⁵, R⁶, and R⁷ are as defined herein.

In certain embodiments, the substituted benzoisochromanequinone of formula (iv) is of the formula (v):

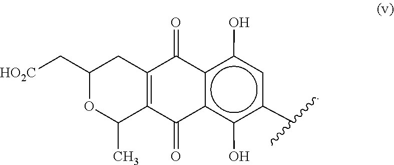

Alternatively, in certain embodiments, R² and R³ are joined to form an optionally substituted carbocyclyl, optionally substituted heterocyclyl, optionally substituted aryl, or optionally substituted heteroaryl group. In certain embodiments, R² and R³ are joined to form an optionally substituted carbocyclyl or optionally substituted heterocyclyl group. In certain embodiments, R² and R³ are joined to form an optionally substituted heterocyclyl group (e.g., an optionally substituted 2-oxabicyclo[2.2.2]octenyl group).

In certain embodiments, R² and R³ are joined to form a substituted 2-oxabicyclo[2.2.2]octenyl group of the formula (vi):

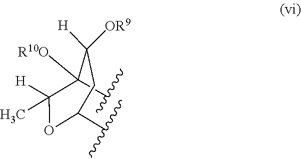

wherein each instance of R⁹ and R¹⁰ is independently selected from hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, carbonyl, silyl, sulfonyl, and sulfinyl.

In certain embodiments, R⁹ is hydrogen. In certain embodiments, R⁹ is optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, R⁹ is optionally substituted carbocyclyl or optionally substituted heterocyclyl. In certain embodiments, R⁹ is optionally substituted heterocyclyl (e.g., optionally substituted tetrahydropyranyl).

In certain embodiments, R⁹ is optionally substituted tetrahydropyranyl group of the formula (vii):

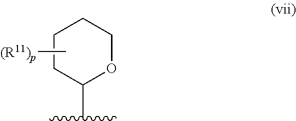

wherein:
each instance of R¹¹ is independently selected from optionally substituted alkyl, optionally substituted alkenyl, optionally substituted alkynyl, optionally substituted carbocyclyl, optionally substituted heterocyclyl, optionally substituted aryl, optionally substituted heteroaryl, —OH, substituted hydroxyl, —SH, substituted thiol, —NH₂, substituted amino, —CN, —NO₂, —N₂, carbonyl, silyl, sulfonyl, and sulfinyl; and
p is 0 or an integer of between 1 and 5, inclusive.

In this instance, in certain embodiments, the R² and R³ are joined to form a substituted 2-oxabicyclo[2.2.2]octenyl group of the formula (viii):

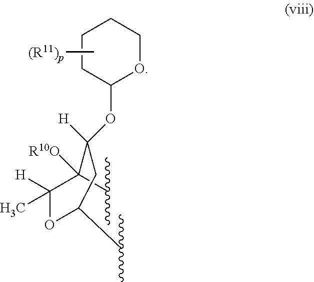

In certain embodiments, each instance of R¹¹ is independently selected from optionally substituted alkyl, —OH, substituted hydroxyl, —SH, substituted thiol, —NH₂, substituted amino, carbonyl, and silyl. In certain embodiments, each instance of R¹⁰ is independently selected from optionally substituted alkyl, —OH, and substituted hydroxyl. In certain embodiments, each instance of R¹¹ is independently selected from optionally substituted alkyl and —OH. In certain embodiments, each instance of R¹¹ is independently selected from —CH₃ and —OH.

In certain embodiments, p is an integer of between 1 and 4, inclusive. In certain embodiments, p is an integer of between 1 and 3, inclusive. In certain embodiments, p is an integer of between 1 and 2, inclusive. In certain embodiments, p is 2.

In certain embodiments, each instance of R¹¹ is independently selected from —CH₃ and —OH, and p is an integer of between 1 and 2, inclusive. For example, in this instance, in certain embodiments, R¹¹ is a substituted tetrahydropyranyl group of the formula (ix):

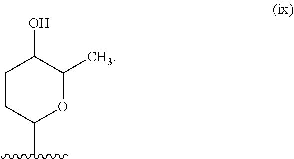 [SMILES: *C1CCC(O)C(C)O1]

In certain embodiments, the substituted tetrahydropyranyl group of the formula (ix) is of the formula (x):

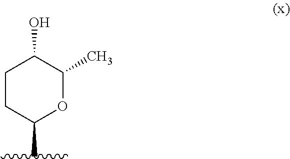 [SMILES: *[C@H]1CC[C@H](O)[C@H](C)O1]

In this instance, in certain embodiments, the R² and R³ are joined to form a substituted 2-oxabicyclo[2.2.2]octenyl group of the formula (xi):

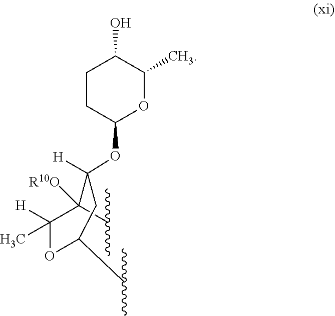

In certain embodiments, R¹⁰ is optionally substituted alkyl, optionally substituted alkenyl, optionally substituted alkynyl, optionally substituted carbocyclyl, optionally substituted heterocyclyl, optionally substituted aryl, optionally substituted heteroaryl, carbonyl, silyl, sulfonyl, or sulfinyl.

However, in certain embodiments, R¹⁰ is hydrogen. In this instance, in certain embodiments, the R² and R³ are joined to form a substituted 2-oxabicyclo[2.2.2]octenyl group of the formula (xii):

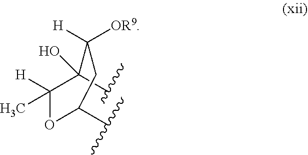

In certain embodiments, both R⁹ and R¹⁰ are hydrogen. In this instance, in certain embodiments, the R² and R³ are joined to form a substituted 2-oxabicyclo[2.2.2]octenyl group of the formula (xiii):

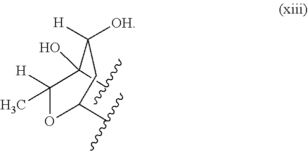 [SMILES: *C1CC(O)C(*)(O)C(C)O1]

In certain embodiments, R⁵ is hydrogen and R⁶ is selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl.

In certain embodiments, both R⁵ and R⁶ are hydrogen. However, in certain embodiments, R⁵ is hydrogen and R⁶ is optionally substituted alkyl, optionally substituted alkenyl, optionally substituted alkynyl, optionally substituted carbocyclyl, optionally substituted heterocyclyl, optionally substituted aryl, optionally substituted heteroaryl.

Alternatively, in certain embodiments, R⁵ and R⁶ are joined to form a direct bond, e.g., to provide a dihydrofuran-2-one of the formula (xiv):

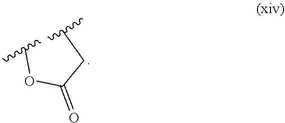 [SMILES: *CC(=O)O*]

In certain embodiments, the dihydrofuran-2-one of the formula (xiv) is of the formula (xv):

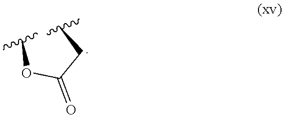

In certain embodiments, the dihydrofuran-2-one of the formula (xiv) is of the formula (xvi):

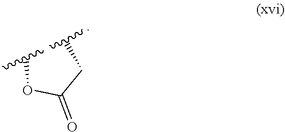

In certain embodiments, R⁷ is hydrogen. In certain embodiments, R⁷ is optionally substituted alkyl, optionally substituted alkenyl, optionally substituted alkynyl, optionally substituted carbocyclyl, optionally substituted heterocyclyl, optionally substituted aryl, and optionally substituted heteroaryl. In certain embodiments, R⁷ is optionally substituted alkyl (e.g., —CH₃, —CH₂CH₃, —CH₂CH₂CH₃, —CH(CH₃)₂, and the like). In certain embodiments, R⁷ is —CH₃.

In certain embodiments, wherein R⁵ and R⁶ are joined to form a direct bond, i.e., to provide a dihydrofuran-2-one of the formula (xiv), the compound of Formula (A) is of the Formula (A-2):

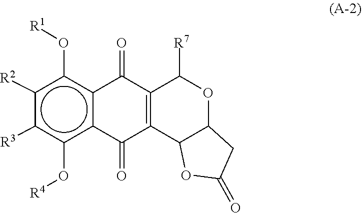

or a pharmaceutically acceptable salt thereof. In certain embodiments, R⁷ is —CH₃. In certain embodiments, R¹ and R⁴ are hydrogen.

In certain embodiments, wherein R⁵ and R⁶ are joined to form a dihydrofuran-2-one of the formula (xv), the compound of Formula (A-2) is of the Formula (A-3):

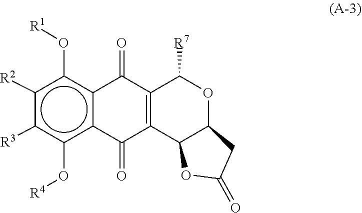

or a pharmaceutically acceptable salt thereof. In certain embodiments, R⁷ is —CH₃. In certain embodiments, R¹ and R⁴ are hydrogen.

In certain embodiments, wherein R⁵ and R⁶ are joined to form a dihydrofuran-2-one of the formula (xvi), the compound of Formula (A-2) is of the Formula (A-4):

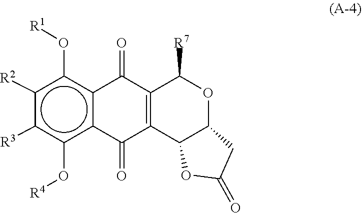

or a pharmaceutically acceptable salt thereof. In certain embodiments, R⁷ is —CH₃. In certain embodiments, R¹ and R⁴ are hydrogen.

In certain embodiments, wherein R¹ and R⁴ are hydrogen, the compound of Formula (A-3) is of the Formula (A-5):

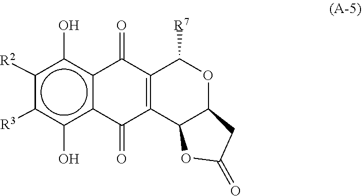

or a pharmaceutically acceptable salt thereof. In certain embodiments, R⁷ is —CH₃.

In certain embodiments, wherein R¹ and R⁴ are hydrogen, the compound of Formula (A-4) is of the Formula (A-6):

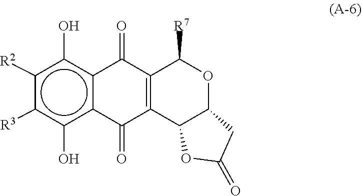

or a pharmaceutically acceptable salt thereof. In certain embodiments, R⁷ is —CH₃.

In certain embodiments, wherein R² and R³ are joined to form a substituted oxabicyclo[2.2.2]octenyl group of the formula (vi), the compound of Formula (A-5) is of the Formula (A-7):

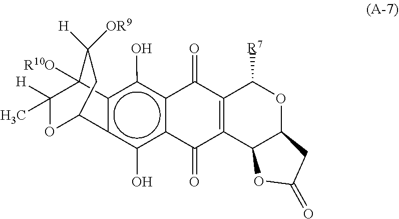

or a pharmaceutically acceptable salt thereof. In certain embodiments, R⁷ is —CH₃.

Exemplary compounds of Formula (A) or (B) comprise granaticin A, granaticin B, dihydrogranaticin A, dihydrogranaticin B, medermycin, and actinorhodin
or pharmaceutically acceptable salts thereof in amorphous form or any crystalline form thereof.

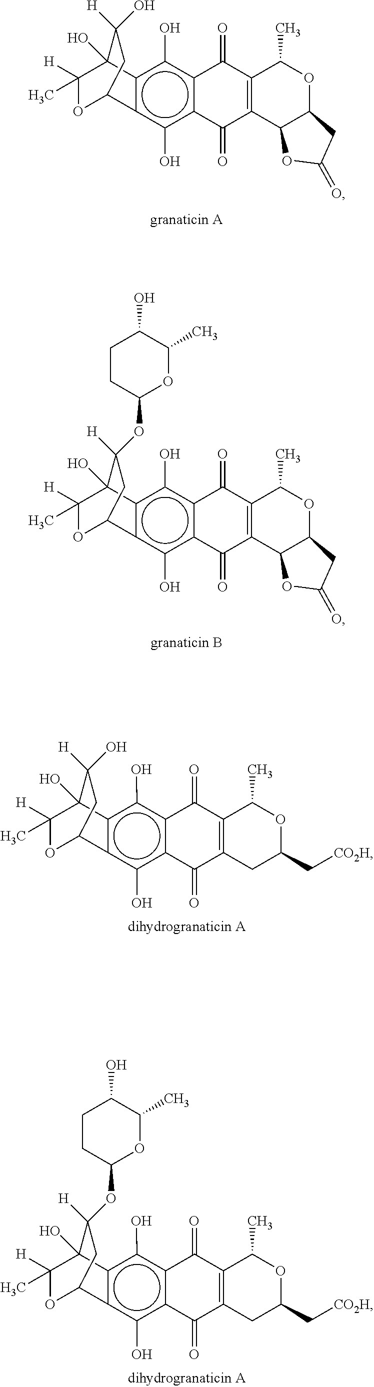

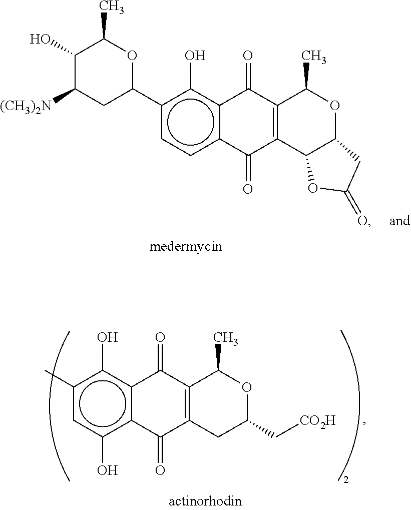

The present invention provides a pharmaceutical composition comprising a combination of (a) a compound of Formula (B) and (b) one or more antineoplastic agents, wherein:

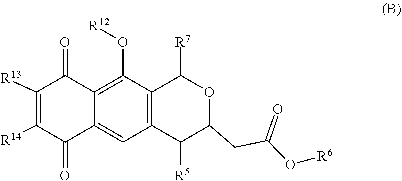

or pharmaceutically acceptable salts thereof,
wherein:
R¹² is hydrogen, carbonyl, silyl, optionally substituted alkyl, optionally substituted alkenyl, optionally substituted alkynyl, optionally substituted carbocyclyl, optionally substituted heterocyclyl, optionally substituted aryl, optionally substituted heteroaryl;
each instance of R¹³ and R¹⁴ is independently selected from the group consisting of hydrogen, halogen, —OH, substituted hydroxyl, —SH, substituted thiol, —NH₂, substituted amino, —CN, —NO₂, carbonyl, silyl, sulfinyl, sulfonyl, optionally substituted alkyl, optionally substituted alkenyl, optionally substituted alkynyl, optionally substituted carbocyclyl, optionally substituted heterocyclyl, optionally substituted aryl, and optionally substituted heteroaryl; or R¹³ and R¹⁴ are joined to form an optionally substituted carbocyclyl, optionally substituted heterocyclyl, optionally substituted aryl, or optionally substituted heteroaryl group;
R⁵ is hydrogen and R⁶ is selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl; or R⁵ and R⁶ are joined to form a direct bond; and
R⁷ is hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl.

In certain embodiments, R¹² is hydrogen. In certain embodiments, R¹² is carbonyl, silyl, optionally substituted carbocyclyl, optionally substituted heterocyclyl, optionally substituted aryl, or optionally substituted heteroaryl.

In certain embodiments, R¹³ is hydrogen. In certain embodiments, R¹³ is halogen, —OH, substituted hydroxyl, —SH, substituted thiol, —NH₂, substituted amino, —CN, —NO₂, carbonyl, silyl, sulfinyl, sulfonyl, optionally substituted alkyl, optionally substituted alkenyl, optionally substituted alkynyl, optionally substituted carbocyclyl, optionally substituted heterocyclyl, optionally substituted aryl, and optionally substituted heteroaryl. In certain embodiments, R¹³ is —OH, substituted hydroxyl, —SH, substituted thiol, —NH₂, substituted amino. In certain embodiments, R¹³ is —OH or substituted hydroxyl. In certain embodiments, R¹³ is —OH.

In certain embodiments, R¹⁴ is hydrogen. In certain embodiments, R¹⁴ is optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, R¹⁴ is optionally substituted carbocyclyl, optionally substituted heterocyclyl, optionally substituted aryl, or optionally substituted heteroaryl. In certain embodiments, R¹⁴ is optionally substituted heterocyclyl (e.g., optionally substituted tetrahydropyranyl).

In certain embodiments, R¹⁴ is an optionally substituted tetrahydropyranyl group of the formula (xvii):

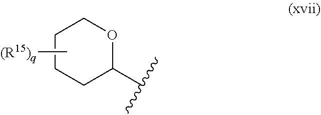

wherein:
each instance of R¹⁵ is independently selected from optionally substituted alkyl, optionally substituted alkenyl, optionally substituted alkynyl, optionally substituted carbocyclyl, optionally substituted heterocyclyl, optionally substituted aryl, optionally substituted heteroaryl, —OH, substituted hydroxyl, —SH, substituted thiol, —NH₂, substituted amino, —CN, —NO₂, carbonyl, silyl, sulfonyl, and sulfinyl; and q is 0 or an integer of between 1 and 5, inclusive.

In certain embodiments, each instance of R¹⁵ is independently selected from optionally substituted alkyl, —OH, substituted hydroxyl, —SH, substituted thiol, —NH₂, substituted amino, carbonyl, and silyl. In certain embodiments, each instance of R¹⁵ is independently selected from optionally substituted alkyl, —OH, substituted hydroxyl, —NH₂, and substituted amino. In certain embodiments, each instance of R¹⁵ is independently selected from optionally substituted alkyl, —OH, and substituted hydroxyl. In certain embodiments, each instance of R¹⁵ is independently selected from —CH₃ and —OH.

In certain embodiments, q is an integer of between 1 and 4, inclusive. In certain embodiments, q is an integer of between 1 and 3, inclusive. In certain embodiments, q is an integer of between 1 and 2, inclusive. In certain embodiments, q is 3.

In certain embodiments, each instance of R¹⁵ is independently selected from —CH₃, —OH, and m is an integer of between 1 and 3, inclusive. For example, in this instance, in certain embodiments, R¹⁵ is a substituted tetrahydropyranyl group of the formula (xviii):

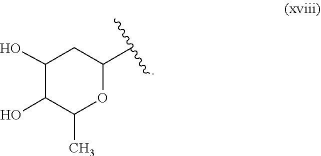 [SMILES: *C1CC(O)C(O)C(C)O1]

Alternatively, in certain embodiments, R¹³ and R¹⁴ are joined to form an optionally substituted carbocyclyl, optionally substituted heterocyclyl, optionally substituted aryl, or optionally substituted heteroaryl group. In certain embodiments, R¹³ and R¹⁴ are joined to form an optionally substituted carbocyclyl or optionally substituted heterocyclyl group. In certain embodiments, R¹³ and R¹⁴ are joined to form an optionally substituted heterocyclyl group (e.g., an optionally substituted 2-oxabicyclo[2.2.2]octenyl group).

In certain embodiments, R¹³ and R¹⁴ are joined to form a substituted 2-oxabicyclo[2.2.2]octenyl group of the formula (xix):

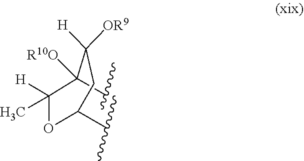

wherein each instance of R¹⁶ and R¹⁷ is independently selected from hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, carbonyl, silyl, sulfonyl, and sulfinyl.

In certain embodiments, R¹⁶ is optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, R¹⁶ is optionally substituted carbocyclyl or optionally substituted heterocyclyl. However, in certain embodiments, R¹⁶ is hydrogen, e.g., and R¹³ and R¹⁴ are joined to form a substituted 2-oxabicyclo[2.2.2]octenyl group of the formula (xx):

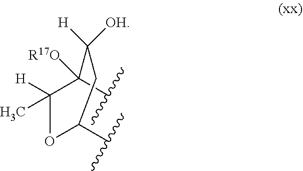

In certain embodiments, R¹⁷ is optionally substituted alkyl, optionally substituted alkenyl, optionally substituted alkynyl, optionally substituted carbocyclyl, optionally substituted heterocyclyl, optionally substituted aryl, or optionally substituted heteroaryl. In certain embodiments, R¹⁷ is optionally substituted carbocyclyl or optionally substituted heterocyclyl. However, in certain embodiments, R¹⁷ is hydrogen, e.g., and R¹³ and R¹⁴ are joined to form a substituted 2-oxabicyclo[2.2.2]octenyl group of the formula (xxi):

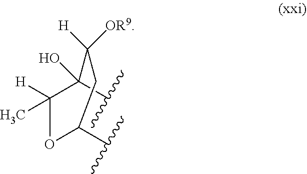

In certain embodiments, both R¹⁶ and R¹⁷ are hydrogen. In this instance, in certain embodiments, the R¹³ and R¹⁴ are joined to form a substituted 2-oxabicyclo[2.2.2]octenyl group of the formula (xxii):

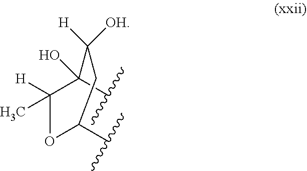 [SMILES: *C1CC(O)C(*)(O)C(C)O1]

In certain embodiments, R⁵ is hydrogen and R⁶ is selected from the group consisting of selected from hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl.

In certain embodiments, both R⁵ and R⁶ are hydrogen. However, in certain embodiments, R⁵ is hydrogen and R⁶ is optionally substituted alkyl, optionally substituted alkenyl, optionally substituted alkynyl, optionally substituted carbocyclyl, optionally substituted heterocyclyl, optionally substituted aryl, optionally substituted heteroaryl.

Alternatively, in certain embodiments, R⁵ and R⁶ are joined to form a direct bond, e.g., to provide a dihydrofuran-2-one of the formula (xiv):

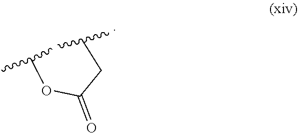 [SMILES: *CC(=O)O*]

In certain embodiments, the dihydrofuran-2-one of the formula (xiv) is of the formula (xv):

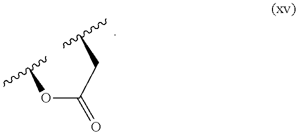

In certain embodiments, the dihydrofuran-2-one of the formula (xiv) is of the formula (xvi):

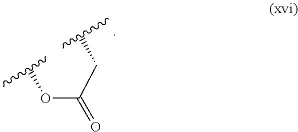

In certain embodiments, R⁷ is hydrogen. In certain embodiments, R⁷ is optionally substituted alkyl, optionally substituted alkenyl, optionally substituted alkynyl, optionally substituted carbocyclyl, optionally substituted heterocyclyl, optionally substituted aryl, and optionally substituted heteroaryl. In certain embodiments, R⁷ is optionally substituted alkyl (e.g., —CH₃, —CH₂CH₃, —CH₂CH₂CH₃, —CH(CH₃)₂, and the like). In certain embodiments, R⁷ is —CH₃.

In certain embodiments, wherein R¹² is hydrogen, the compound of Formula (B) is of the Formula (B-1):

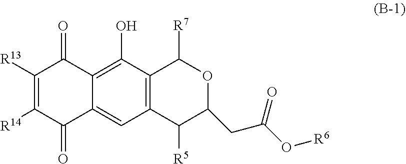

or a pharmaceutically acceptable salt thereof. In certain embodiments, R⁷ is —CH₃.

In certain embodiments, wherein R⁵ and R⁶ are joined to form a direct bond, the compound of Formula (B-1) is of the Formula (B-2):

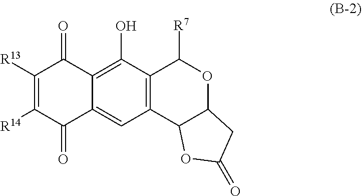

or a pharmaceutically acceptable salt thereof. In certain embodiments, R⁷ is —CH₃.

In certain embodiments, wherein R¹³ and R¹⁴ are joined to form a substituted 2-oxabicyclo[2.2.2]octenyl group of the formula (xix), the compound of Formula (B-1) is of the Formula (B-3):

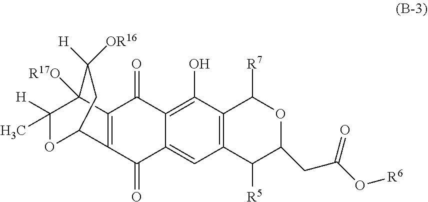

or a pharmaceutically acceptable salt thereof. In certain embodiments, R⁷ is —CH₃.

In certain embodiments, wherein R¹³ is —OH and R¹⁴ is an optionally substituted tetrahydropyranyl group of the formula (xvii), the compound of Formula (B-1) is of the Formula (B-4):

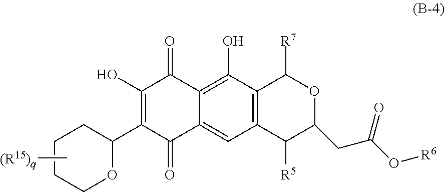

or a pharmaceutically acceptable salt thereof. In certain embodiments, R⁷ is —CH₃.

Exemplary compounds of the Formula (B) include, but are not limited to:

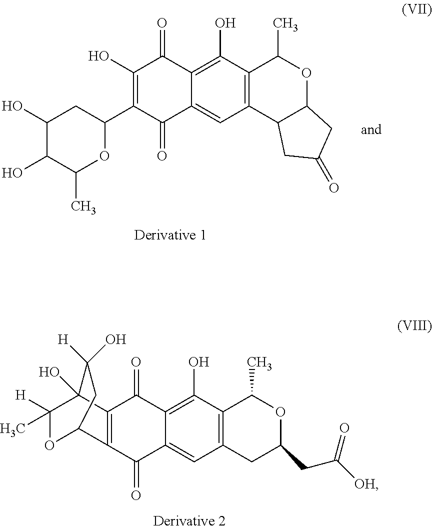 [SMILES: CC1OC2CC(=O)CC2c2cc3c(c(O)c21)C(=O)C(O)=C(C1CC(O)C(O)C(C)O1)C3=O ; CC1OC2CC(O)C1(O)C1=C2C(=O)c2cc3c(c(O)c2C1=O)[C@H](C)O[C@@H](CC(=O)O)C3]

or pharmaceutically acceptable salts thereof.

In certain embodiments, the compounds of Formula (A) and (B) have an IC₅₀ of less than approximately 100 μM, e.g., less than approximately 10 μM, e.g., less than approximately 1 μM, e.g., less than approximately 0.1 μM, or e.g., less than approximately 0.01 μM.

The compound of Formula (A) or (B) useful in the present invention may exist in particular geometric or stereoisomeric forms. The present invention contemplates all such compounds, including cis-and trans-isomers, R- and S-enantiomers, diastereomers, (D)-isomers, (L)-isomers, the racemic mixtures thereof, and other mixtures thereof, as falling within the scope of the invention. As used herein, the term “isomers” includes any and all geometric isomers and stereoisomers. For example, “isomers” include cis-and trans-isomers, E-and Z-isomers, R-and S-enantiomers, diastereomers, (D)-isomers, (L)-isomers, racemic mixtures thereof, and other mixtures thereof, as falling within the scope of the invention. For instance, an isomer/enantiomer may, in some embodiments, be provided substantially free of the corresponding enantiomer, and may also be referred to as “optically enriched.” “Optically enriched,” as used herein, means that the compound is made up of a significantly greater proportion of one enantiomer. In certain embodiments the compound of the present invention is made up of at least about 90% by weight of a particular enantiomer. In other embodiments the compound is made up of at least about 95%, 98%, or 99% by weight of a particular enantiomer. A desired enantiomer may be isolated from a racemic mixture by any method known to those skilled in the art, including chiral high pressure liquid chromatography (HPLC) or the formation and crystallization of chiral salts, or the enantiomer may be prepared by asymmetric syntheses. See, for example, Jacques, et al., Enantiomers, Racemates and Resolutions (Wiley Interscience, New York, 1981); Wilen, S. H., et al., Tetrahedron 33:2725 (1977); Eliel, E. L. Stereochemistry of Carbon Compounds (McGraw-Hill, NY, 1962); Wilen, S. H. Tables of Resolving Agents and Optical Resolutions p. 268 (E. L. Eliel, Ed., Univ. of Notre Dame Press, Notre Dame, Ind. 1972).

In an embodiment, the compound of Formula (A) or (B) comprises granaticin B or a pharmaceutically acceptable salt thereof in amorphous form or any of its crystalline forms. In an embodiment, the compound of Formula (A) or (B) consists of granaticin B or a pharmaceutically acceptable salt thereof in amorphous or any of its crystalline forms. In an embodiment, the compound of Formula (A) or (B) comprises crystalline Form A of granaticin B. In an embodiment, the crystalline Form A of granaticin B is characterized by X-ray powder diffraction pattern comprising at least 4, 5, 7, 9, 13, or 17 peaks of 9.1±0.2, 10.0±0.2, 11.6±0.2, 16.1±0.2, 18.3±0.2, 4.5±0.2, 15.4±0.2, 12.6±0.2, 17.0±0.2, 13.3±0.2, 17.5±0.2, 22.1±0.2, 23.3±0.2, 8.7±0.2, 19.7±0.2, 20.3±0.2, and 21.6±0.2 2-Theta°. Various crystalline forms of granaticin B are described in U.S. Pat. No. 10,314,815 which is hereby incorporated by reference in its entirety.

In some embodiments, a particular crystalline form of granaticin B is substantially free of impurities. As used herein, impurities include, but are not limited to, any extraneous matter such residual solvents, salts, or other forms of granaticin B. In some embodiments, a particular crystalline form of granaticin B is 99% free of impurities. In some embodiments, a particular crystalline form of granaticin B is 97% free of impurities. In some embodiments, a particular crystalline form of granaticin B is 95% free of impurities. In some embodiments, a particular crystalline form of granaticin B is 92% free of impurities. In some embodiments, a particular crystalline form of granaticin B is 90% free of impurities. In certain embodiments, the impurities include extraneous matter, such as a salt forming acid, residual solvents, or any other impurities that may result from the preparation and/or isolation of granaticin B. In some embodiments, a particular crystalline form of granaticin B is substantially free of amorphous form of granaticin B. In some embodiments, a particular crystalline form of granaticin B is substantially free of any other crystalline form of granaticin B. In some embodiments, a particular crystalline form of granaticin B is substantially free of a salt of granaticin B. In some embodiments, a particular crystalline form of granaticin B is substantially free of a solvate of granticin B.

In some embodiments, Form A is substantially free of impurities. As used herein, impurities include, but are not limited to, any extraneous matter such residual solvents, salts, or other forms of granaticin B. In some embodiments, Form A is 99% free of impurities. In some embodiments, Form A is 97% free of impurities. In some embodiments, Form A is 95% free of impurities. In some embodiments, Form A is 92% free of impurities. In some embodiments, Form A is 90% free of impurities. In certain embodiments, the impurities include extraneous matter, such as a salt forming acid, residual solvents, or any other impurities that may result from the preparation and/or isolation of granaticin B. In some embodiments, Form A is substantially free of amorphous granaticin B. In some embodiments, Form A is substantially free of another crystalline form of granaticin B. In some embodiments, Form A is substantially free of a salt of granaticin B. In some embodiments, Form A is substantially free of a solvate of granaticin B.

In an embodiment, the compound of Formula (A) or (B) of the present invention is an inhibitor of cell replication cycle. In an embodiment, the compound of Formula (A) or (B) is an inhibitor of cell replication cycle at G1/S phase. In an embodiment, the compound of Formula (A) or (B) is an inhibitor of DNA replication during cell cycle. In an embodiment, the inhibition of cell cycle or the inhibition of DNA replication during the cell cycle by compound of Formula (A) or (B) suppresses abnormal cell proliferation in a subject with a cell proliferation disorder. In an embodiment, the inhibition of cell cycle or the inhibition of DNA replication during the cell cycle by compound of Formula (A) or (B) is useful in suppressing an abnormal cell proliferation of a subject with a proliferation disorder without affecting the proliferation of a normal cell of the subject.

In an embodiment, the compound of Formula (A) or (B) of the present invention is an inhibitor of a protein kinase. In an embodiment, the compound of Formula (A) or (B) is an inhibitor of cell division cycle 7 (CDC7) kinase. In an embodiment, the compound of Formula (A) or (B) is a non-ATP-competitive inhibitor of CDC7 kinase. In an embodiment, the inhibition of CDC7 kinase involves inhibition of the Dbf4 subunit of CDC7 kinase, wherein the inhibition of CDC7 kinase or the Dbf4 subunit of CDC7 kinase is useful for the treatment of a proliferation disorder.

In an embodiment, the pharmaceutical composition of the present invention comprises a combination of (a) any embodiment of the compound of Formula (A) or (B) and (b) one or more antineoplastic agents, wherein the one or more antineoplastic agents comprises hydroxyurea, BCL2 inhibitor, hypomethylating agent and/or other chemotherapy drugs. In an embodiment, BCL2 inhibitor comprises Venetoclax, BH3 mimetics or a combination thereof. In an embodiment, the hypomethylation agent comprises azacytidine, decitabine or a combination thereof. In an embodiment, the chemotherapeutic agent comprises cytarabine, daunorbicin, anthracycline or a combination thereof. In an embodiment, the one or more antineoplastic agents of the pharmaceutical composition of the present invention are each in free form, in the form of a pharmaceutically acceptable salt and/or a hydrate thereof.

Other exemplary chemotherapeutic agents include, but are not limited to, anti-estrogens (e.g. tamoxifen, raloxifene, and megestrol), LHRH agonists (e.g. goscrclin and leuprolide), anti-androgens (e.g. flutamide and bicalutamide), photodynamic therapies (e.g. vertoporfin (BPD-MA), phthalocyanine, photosensitizer Pc4, and demethoxy-hypocrellin A (2BA-2-DMHA)), nitrogen mustards (e.g. cyclophosphamide, ifosfamide, trofosfamide, chlorambucil, estramustine, and melphalan), nitrosoureas (e.g. carmustine (BCNU) and lomustine (CCNU)), alkylsulphonates (e.g. busulfan and treosulfan), triazenes (e.g. dacarbazine, temozolomide), platinum containing compounds (e.g. cisplatin, carboplatin, oxaliplatin), vinca alkaloids (e.g. vincristine, vinblastine, vindesine, and vinorelbine), taxoids (e.g. paclitaxel or a paclitaxel equivalent such as nanoparticle albumin-bound paclitaxel (ABRAXANE), docosahexaenoic acid bound-paclitaxel (DHA-paclitaxel, Taxoprexin), polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, e.g., 2′-paclitaxel methyl 2-glucopyranosyl succinate; docetaxel, taxol), epipodophyllins (e.g. etoposide, etoposide phosphate, teniposide, topotecan, 9-aminocamptothecin, camptoirinotecan, irinotecan, crisnatol, mytomycin C), anti-metabolites, DHFR inhibitors (e.g. methotrexate, dichloromethotrexate, trimetrexate, edatrexate), IMP dehydrogenase inhibitors (e.g. mycophenolic acid, tiazofurin, ribavirin, and EICAR), ribonuclotide reductase inhibitors (e.g. hydroxyurea and deferoxamine), uracil analogs (e.g. 5-fluorouracil (5-FU), floxuridine, doxifluridine, ratitrexed, tegafur-uracil, capecitabine), cytosine analogs (e.g. cytarabine (ara C), cytosine arabinoside, and fludarabine), purine analogs (e.g. mercaptopurine and Thioguanine), Vitamin D3 analogs (e.g. EB 1089, CB 1093, and KH 1060), isoprenylation inhibitors (e.g. lovastatin), dopaminergic neurotoxins (e.g. 1-methyl-4-phenylpyridinium ion), cell cycle inhibitors (e.g. staurosporine), actinomycin (e.g. actinomycin D, dactinomycin), bleomycin (e.g. bleomycin A2, bleomycin B2, peplomycin), anthracycline (e.g. daunorubicin, doxorubicin, pegylated liposomal doxorubicin, idarubicin, epirubicin, pirarubicin, zorubicin, mitoxantrone), MDR inhibitors (e.g. verapamil), Ca²⁺ ATPase inhibitors (e.g. thapsigargin), imatinib, thalidomide, lenalidomide, tyrosine kinase inhibitors (e.g., axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228), proteasome inhibitors (e.g., bortezomib (VELCADE)), mTOR inhibitors (e.g., rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI)), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine.

Other chemotherapeutic drugs may comprise Gemtuzumab, ozogamicin, Idarubicin, CPX-351, Enasidenib (IDH2 inibitor), FTL3 inhibitors (Sunitinib, Midostaurin, Lestaurtinib, Crenolanib, Gilteritinib, Sorafenib, Ponatinib, Quizartinib), Ivosidenib (IDH1 inhibitor), Venetoclax (BCL-2 inhibitor) and Glasdegib, rituximab, ofatuzumab (Anti-CD20), inotuzumab ozogamicin (Anti-CD22), blinatumomab (Anti-CD19), and Tisagenlecleucel, tyrosine kinase inhibitor (Imatinib, Dasatinib, Nilotinib, Ponatinib, Bosatinib, Asciminib) with/without chemotherapy (Clofarabine, Nelarabine, Liposomal vincristine, MOpAD, Hyper-CVAD, BFM, etc) or steroid, inotuzumab ozogamicin, blinatumomab, and Tisagenlecleucel.

In an embodiment, the pharmaceutical composition of the present invention further comprises a pharmaceutically acceptable excipient. Pharmaceutically acceptable excipients include any solvents, diluents or other liquid vehicles, dispersion or suspension aids, surface active agents, isotonic agents, thickening or emulsifying agents, preservatives, solid binders, lubricants and the like, as suited to the particular dosage form desired. In an embodiment, excipients such as cocoa butter and suppository waxes, coloring agents, coating agents, sweetening, flavoring, and perfuming agents may also be present in the composition. General considerations in the formulation and/or manufacture of pharmaceutical compositions agents can be found, for example, in Remington's Pharmaceutical Sciences, Sixteenth Edition, E. W. Martin (Mack Publishing Co., Easton, Pa., 1980), and Remington: The Science and Practice of Pharmacy, 21st Edition (Lippincott Williams & Wilkins, 2005).

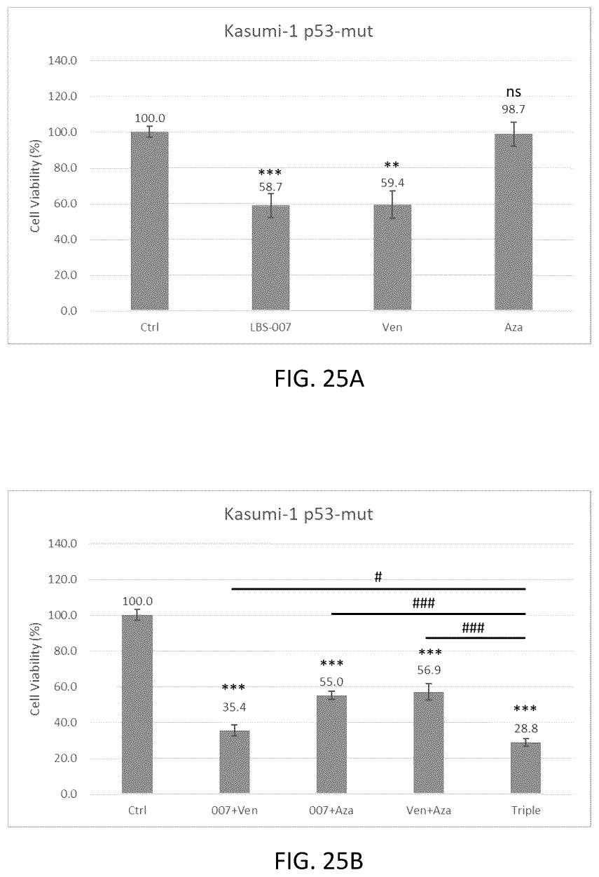

As shown in Example 5 in connection with FIG. 25 for cell viability experiment on Kasumi-1 cells, azicitydine (Aza) alone had almost no effect, and LBS-007 and Venetoclax (Ven) each alone provided about the same effectiveness on Kasumi-1 cells with cell viability at about 60%. Notably, combination of Aza+Ven as well as LBS-007+Aza provided no additional benefit as compared to LBS-007 or Ven alone, illustrating that double combination therapy do not always result in better results. However, when LBS-007 is combined with Ven, cell viability substantially decreased by almost half to about 35%, demonstrating that the LBS-007 and Ven combination provides synergistic effect. Surprisingly, although Aza alone is shown to have no effect on cell viability, the addition of Aza to LBS-007 and Ven provided nearly 20% improvement to cell viability results.

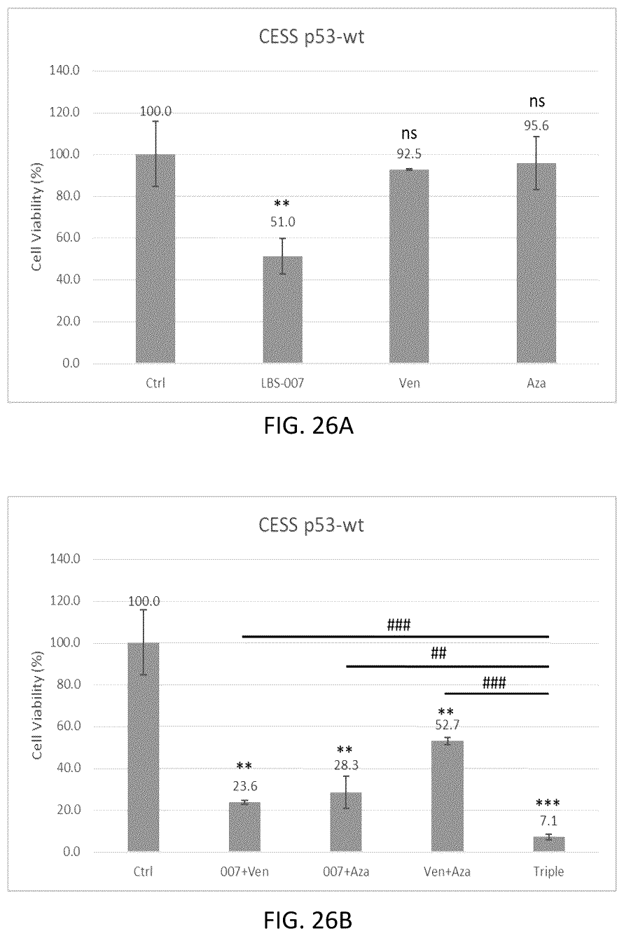

As shown in Example 5 in connection with FIG. 26 for cell viability experiment on CESS cells, Ven and Aza alone showed little or no effect on cell viability at the concentration tested whereas LBS-007 demonstrated substantial effect on cell viability at about 50%. Although showing no effect individually, Ven+Aza in combination achieved about 50% cell viability. However, the combinations LBS-007+Aza and LBS-007+Ven doubled effect on cell viability compared to that of the Ven+Aza combination to about 25% cell viability. Most notably, treatment of the CESS cells with the triple combination of LBS-007+Aza+Ven resulted in cell viability of about 7%.

The Examples demonstrated that therapy with certain combinations of compounds work substantially better than others and that the triple treatment approach especially combining LBS-007, Venetoclax (Ven), and Azacitidine (Aza) in particular proved to be the most effective in inducing cytotoxicity in these acute myeloid leukemia (AML) cell lines. This finding underscores the potential of certain combination treatment as effective therapeutic avenue for AML.

Therefore, the present invention provides a method of treatment for AML comprising the step of administration of any embodiment of the compound of formula A or B of the present invention disclosed herein in combination with Aza to a subject suffering from AML. The present invention also provides a method of treatment for AML comprising the step of administration of any embodiment of the compound of formula A or B of the present invention disclosed herein in combination with Ven to a subject suffering from AML. In addition, the present invention provides a method of treatment for AML comprising the step of administration of any embodiment of the compound of formula A or B in combination with Aza and Ven to a subject suffering from AML. In an embodiment, the compound of formula A or B comprises graniticin B. In an embodiment, the compound of formula A or B comprises amorphous form of graniticin B. In an embodiment, the compound of formula A or B comprises any crystalline form of graniticin B. In an embodiment, the compound of formula A or B comprises crystalline A form of graniticin B. Various embodiments of compound of formula (A) and (B) are described herein as well as in U.S. Pat. No. 9,180,105 which is hereby incorporated by reference in its entirety.

The present invention also provides a method of treating a proliferation disorder of a subject comprising the administration of a combination of (a) any embodiment of the compound of Formula (A) or (B) and (b) one or more of any embodiments of the antineoplastic agent of the present invention disclosed herein.

In an embodiment, the method of treating a proliferation disorder of the present invention comprising the administration of a combination of (a) any embodiment of the compound of Formula (A) or (B) and (b) one or more of any embodiment of the antineoplastic agents of the present invention wherein the (a) any embodiment of the compound of Formula (A) or (B) and (b) the one or more of any embodiment of the antineoplastic agents of the present invention is administered to the subject simultaneously, separately, or sequentially. In an embodiment, the any of the embodiment of the compound of Formula (A) or (B) is administered to a subject before or after the administration of the one or more any embodiment of the antineoplastic agents to the subject. In an embodiment, the any of the embodiment of the compound of Formula (A) or (B) is administered to a subject simultaneously with the administration of the one or more antineoplastic agents to the subject. In an embodiment, the method of treating a proliferation disorder of the present invention comprises the administration of a combination of (a) any embodiment of the compound of Formula (A) or (B) and (b) one of any embodiment of the antineoplastic agents of the present invention. In an embodiment, the method of treating a proliferation disorder of the present invention comprises the administration of a combination of (a) any embodiment of the compound of Formula (A) or (B) and (b) two of any embodiment of the antineoplastic agents of the present invention.

In an embodiment, the one or more antineoplastic agents comprises azacitidine and venetoclax. In an embodiment, the one or more antineoplastic agents comprises azacitidine. In an embodiment, the one or more antineoplastic agents comprises venetoclax. In an embodiment, the one or more antineoplastic agent comprises fludarabine, arabinofuranosyl cytidine, granulocyte colony-stimulating factor and idarubicin (FLAG-IDA) and wherein administration step comprises the standard FLAG-IDA chemotherapy regimen administered in combination with the compound of Formula (A) or (B). In an embodiment, the one or more antineoplastic agent comprises FLAG-IDA and venetoclax and wherein the administration step comprises the standard FLAG-IDA chemotherapy regimen administered in combination with the compound of Formula (A) or (B). In an embodiment, the one or more antineoplastic agent comprises cytarabine and anthracycline antibiotic and wherein the administration step comprises the standard 7 days of standard-dose cytarabine followed by 3 days of anthracycline antibiotic chemotherapy regimen administered in combination with the compound of Formula (A) or (B). In an embodiment, the one or more antineoplastic agent comprises gilteritinib and azacitidine. In an embodiment, the one or more antineoplastic agent comprises enasidenib and azacitidine. In an embodiment, the one or more antineoplastic agent comprises HiDAC and gileritinib. In an embodiment, the one or more antineoplastic agent comprises oral azacitidine. In an embodiment, the one or more antineoplastic agent comprises ponatinib and standard induction chemotherapy and blinatumomab. In an embodiment, the standard induction chemotherapy comprises any standard induction chemotherapy known in the art. In an embodiment, the standard induction chemotherapy comprises the 7+3 regimen wherein the 7+3 regimen coprises administration of cytarabine continuously for 7 days along with short infusions of an anthracycline on each of the first 3 days. In an embodiment, the one or more antineplastic agent comprises inotuzumab ozogamicin and conventional chemotherapy. In an embodiment, conventional chemotherapy comprises administration of any antineoplastic compound disclosed here or a combination thereof. In an embodiment, the conventional chemotherapy comprises any standard induction chemotherapy known in the art.

In an embodiment, the compound of Formula (A) or (B) comprises granticin B and the one or more antineoplastic agents comprises azacitidine and Venetoclax. In an embodiment, the compound of Formula (A) or (B) comprises granticin B and the one or more antineoplastic agents comprises azacitidine. In an embodiment, the one or more antineoplastic agents comprises Venetoclax. In an embodiment, the compound of Formula (A) or (B) comprises granticin B and the one or more antineoplastic agent comprises fludarabine, arabinofuranosyl cytidine, granulocyte colony-stimulating factor and idarubicin (FLAG-IDA) and wherein administration step comprises the standard FLAG-IDA chemotherapy regimen administered in combination with the compound of Formula (A) or (B). In an embodiment, the compound of Formula (A) or (B) comprises granticin B and the one or more antineoplastic agent comprises FLAG-IDA and venetoclax and wherein administration step comprises the standard FLAG-IDA chemotherapy regimen administered in combination with the compound of Formula (A) or (B). In an embodiment, the compound of Formula (A) or (B) comprises granticin B and the one or more antineoplastic agent comprises cytarabine and anthracycline antibiotic and wherein administration step comprises the standard 7 days of standard-dose cytarabine followed by 3 days of anthracycline antibiotic chemotherapy regimen administered in combination with the compound of Formula (A) or (B). In an embodiment, the 7 day standard-dose cytarabine comprises 100 mg/m2/day by continuous IV infusion (Days 1 through 7) or 100 mg/m2 IV every 12 hours (Days 1 through 7). In an embodiment, the compound of Formula (A) or (B) comprises granticin B and the one or more antineoplastic agent comprises gilteritinib and azacitidine. In an embodiment, the compound of Formula (A) or (B) comprises granticin B and the one or more antineoplastic agent comprises enasidenib and azacitidine. In an embodiment, the compound of Formula (A) or (B) comprises granticin B and the one or more antineoplastic agent comprises HiDAC and gileritinib. In an embodiment, the compound of Formula (A) or (B) comprises granticin B and the one or more antineoplastic agent comprises oral azacitidine. In an embodiment, the compound of Formula (A) or (B) comprises granticin B and the one or more antineoplastic agent comprises ponatinib and standard induction chemotherapy and blinatumomab. In an embodiment, the standard induction chemotherapy comprises any standard induction chemotherapy known in the art. In an embodiment, the standard induction chemotherapy comprises the 7+3 regimen wherein the 7+3 regimen coprises administration of cytarabine continuously for 7 days along with short infusions of an anthracycline on each of the first 3 days. In an embodiment, the compound of Formula (A) or (B) comprises granticin B and the one or more antineplastic agent comprises inotuzumab ozogamicin and conventional chemotherapy. In an embodiment, convention chemotherapy comprises administration of any antineoplastic compound disclosed here or a combination thereof. In an embodiment, the conventional chemotherapy comprises any standard induction chemotherapy known in the art

In an embodiment, the proliferation disorder comprises cancer, myeloproliferative disorders, benign prostate hyperplasia, familial adenomatosis, polyposis, neuro-fibromatosis, psoriasis, vascular smooth cell proliferation associated with atherosclerosis, fibrotic disorders, pulmonary fibrosis, arthritis, rheumatoid arthritis, glomerulonephritis, and post-surgical stenosis, restenosis, disorders of proliferation of blood vessels, disorders of proliferation of mesangial cells, metabolic disorders, allergies, asthmas, thromboses, diseases of the nervous system, retinopathy, diabetes, and muscular degeneration.

In an embodiment, the proliferative disorder comprises cancer, wherein the cancer is originated from at least a part of a subject body comprising bone, brain, connective tissue, endocrine glands, adrenal cortex, endometrium, germ cells, head and neck, larynx and hypopharynx, mesothelioma, muscle, rectum, renal, small intestine, soft tissue, testis, ureter, vagina, and vulva. In an embodiment, the proliferative disorder comprises cancer, wherein the cancer comprises bladder cancer, breast cancer, colon cancer, kidney cancer, liver cancer, lung cancer, esophagus cancer, gallbladder cancer, ovarian cancer, pancreatic cancer, stomach cancer, cervical cancer, thyroid cancer, prostate cancer, papillary thyroid carcinoma, genitourinary malignancies, retinoblastoma, Wilms tumor, myelodysplastic syndrome, plasma cell neoplasia, paraneoplastic syndromes, renal cell carcinoma, Ewing's sarcoma, desmoplastic small round cell tumors, mesothelioma, skin cancer comprising squamous cell carcinoma, hematologic cancers comprising leukemia, acute lymphocytic leukemia (ALL), acute lymphoblastic leukemia, lymphoma, B-cell lymphoma, T-cell lymphoma, Hodgkin's lymphoma, mantle cell lymphoma (MCL), hairy cell lymphoma, Burkitt's lymphoma, chronic lymphocytic leukemia (CLL), multiple myeloma, chronic myeloid leukemia (CML), acute myeloid leukemia (AML), myelodysplastic syndrome, promyelocytic leukemia, fibrosarcoma, rhabdomyosarcoma, astrocytoma, neuroblastoma, glioma, schwannomas, melanoma, cutaneous melanoma, seminoma, teratocarcinoma, osteosarcoma, xeroderma pegmentosum, keratoxanthoma, thyroid follicular cancer, Kaposi's sarcoma, cancers of unknown primary site, solid tumors, hematologic cancers, and AIDS-related malignancies.

In certain embodiments, the cancer is a multi-drug resistant (MDR) cancer such as AML or TKI (tyrosine kinase inhibitor)-resistant Ph+ ALL (Philadelphia chromosome positive acute lymphoblastic leukemia). In certain embodiments, the cancer is relapsed and/or refractory cancer. Relapsed cancer refers to a cancer which has returned after a patient has enjoyed a remission. Refractory cancer refers to a cancer which does not respond to other therapies or therapeutic agents. In certain embodiments, the cancer is resistant to (i.e., does not respond to) therapies or chemotherapeutic agents. In certain embodiments, the hematologic cancer is resistant to therapies or chemotherapeutic agents.

In certain embodiments, the cancer is leukemia, lymphoma, melanoma, cancer of the breast, stomach, ovaries, colon, rectum, lung, brain, larynx, lymphatic system, thyroid, oesophagus, liver, uterus, testis, bladder, prostate, bones or pancreas. In certain embodiments, the cancer is leukemia, cancer of the breast, the colon or the lung.

In certain embodiments, the cancer is a hematologic cancer. In certain embodiments, the compound inhibits the growth of hematologic cancers. In certain embodiments, the compound inhibits the growth of hematologic cancers with IC50s in the nanomolar range. In certain embodiments, the hematologic cancer is a hematopoietic cancer of lymphoid lineage. In certain embodiments, the hematologic cancer is a relapsed and/or refractory hematopoietic cancer of lymphoid lineage. In certain embodiments, the cancer is refractory to multiple cycles of cancer therapy (e.g., including allogenic bone marrow transplantation). In certain embodiments, the hematologic cancer is relapsed and/or refractory ALL, CLL, AML, or CML.

In certain embodiments, the cancer is ovarian cancer. In certain embodiments, the ovarian cancer is ovarian carcinoma.

In certain embodiments, the cancer is lung cancer. In certain embodiments, the lung cancer is non-small cell lung cancer. In certain embodiments, the lung cancer is small cell lung cancer. In certain embodiments, the lung cancer is lung adenocarcinoma.

In certain embodiments, the cancer is prostate cancer.

In certain embodiments, the cancer is renal cell carcinoma.

In certain embodiments, the cancer is cervical cancer. In certain embodiments, the cervical cancer is cervical adenocarcinoma. In certain embodiments, the cervical cancer is positive for human papillomavirus (HPV) infection.

In certain embodiments, the cancer is glioblastoma.

In certain embodiments, the cancer is retinoblastoma.

In certain embodiments, the cancer is rhabdomyosarcoma.

In certain embodiments, the cancer is a desmoplastic small round cell tumor.

In certain embodiments, the cancer is breast cancer. In certain embodiments, the breast cancer is breast ductal carcinoma. In certain embodiments, the breast cancer is breast adenocarcinoma. In certain embodiments, the breast cancer is metastatic breast adenocarcinoma. In certain embodiments, the breast cancer is HER2 negative. In certain embodiments, the breast cancer is HER2 positive. In certain embodiments, the breast cancer is NEU receptor negative.

In certain embodiments, the cancer is mesothelioma.

In certain embodiments, the cancer is melanoma.

In certain embodiments, the cancer is thyroid carcinoma.

In certain embodiments, the cancer is Ewing's sarcoma.

In certain embodiments, the cancer is a solid tumor. In certain embodiments, the compound inhibits the growth of solid tumors. In certain embodiments, the compound inhibits the growth of solid tumors with IC50s in the nanomolar range.

In certain embodiments, the cancer comprises a genetic mutation.

In certain embodiments, the cancer comprises a RAS mutation. In certain embodiments, the cancer comprises wild-type RAS.

In certain embodiments, cancer comprises an EGFR mutation. In certain embodiments, the EGFR mutation is an L858R EGFR mutation. In certain embodiments, the EGFR mutation is an DelE746 EGFR mutation. In certain embodiments, the EGFR mutation is an DelE746-A750 EGFR mutation. In certain embodiments, the EGFR mutation is an DelE746-E749 EGFR mutation. In certain embodiments, the EGFR mutation is an T790M EGFR mutation. In certain embodiments, the EGFR mutation is an T790M/L858R EGFR mutation. In certain embodiments, the cancer comprises wild-type EGFR.

In certain embodiments, the cancer comprises a KRAS mutation. In certain embodiments, the cancer comprises a G13C KRAS mutation. In certain embodiments, the cancer comprises a G12C KRAS mutation. In certain embodiments, the cancer comprises a G12C KRAS mutation. In certain embodiments, the cancer comprises a Q61H KRAS mutation. In certain embodiments, the cancer comprises wild-type KRAS.

In certain embodiments, the cancer comprises a p53 mutation. In certain embodiments, the p53 mutation is a R273H p53 mutation. In certain embodiments, the p53 mutation is a G262V p53 mutation. In certain embodiments, the p53 mutation is a G16L p53 mutation. In certain embodiments, the p53 mutation is a C176F p53 mutation. In certain embodiments, the p53 mutation is a M246I p53 mutation. In certain embodiments, the cancer comprises wild-type p53.

In certain embodiments, the cancer comprises a BRAF mutation. In certain embodiments, the BRAF mutation is a BRAF V600E mutation.

In certain embodiments, the cancer comprises a EVI1 mutation.

In certain embodiments, the cancer comprises a Flt-3 mutation.

In certain embodiments, the cancer comprises a WT-1 mutation.

In certain embodiments, the cancer comprises a cyclin D mutation.

In certain embodiments, the cancer comprises a PTEN mutation.

In certain embodiments, the cancer comprises a ABL kinase mutation.

In certain embodiments, the mutation comprises a chromosomal abnormality. In

certain embodiments, the chromosomal abnormality is a chromosome deletion or inversion.

In certain embodiments, the cancer comprises a chromosome 17p deletion. In certain embodiments, the cancer comprises an inversion of chromosome 16. In certain embodiments, the cancer comprises a trisomy of chromosome 8. In certain embodiments, the cancer comprises a monosomy of chromosome 7. In certain embodiments, the cancer comprises a chromosome 11q23 abnormality. In certain embodiments, the cancer comprises a Philadelphia chromosome positive abnormality.

In certain embodiments, the cancer comprises a fusion transcript. In certain embodiments, the fusion transcript is a reciprocal ASPL-TFE3 fusion transcript.

In an embodiment, the method of treating a proliferation disorder of the present invention can be administered in combination with additional cancer therapies that improves their bioavailability, reduce and/or modify their metabolism, inhibit their excretion, and/or modify their distribution within the body. In an embodiment, the additional cancer therapy comprises surgical treatments, radiation therapy, and therapeutic agents. In an embodiment, the additional cancer therapy is administered to a subject before or after the method of treating a proliferation disorder to the subject of the present invention. In an embodiment, the additional cancer therapy is administered to a subject simultaneously with the method of treating a proliferation disorder to the subject of the present invention.

In certain embodiments, a therapeutically effective amount of any embodiment of the compound of Formula (A) or (B) or any embodiment of the antineoplastic agent for administration one or more times a day to a 70 kg adult human may comprise about 0.0001 mg to about 3000 mg, about 0.0001 mg to about 2000 mg, about 0.0001 mg to about 1000 mg, about 0.001 mg to about 1000 mg, about 0.01 mg to about 1000 mg, about 0.1 mg to about 1000 mg, about 1 mg to about 1000 mg, about 1 mg to about 100 mg, about 10 mg to about 1000 mg, or about 100 mg to about 1000 mg, about 0.001 mg to about 100 mg, from about 0.01 mg to about 50 mg, from about 0.1 mg to about 40 mg, from about 0.5 mg to about 30 mg, from about 0.01 mg to about 10 mg, from about 0.1 mg to about 10 mg, and from about 1 mg to about 25 mg, per kilogram, of a compound. It will be appreciated that dose ranges as described herein provide guidance for the administration of provided pharmaceutical compositions to an adult. The amount to be administered to, for example, a child or an adolescent can be determined by a medical practitioner or person skilled in the art and can be lower or the same as that administered to an adult. The desired dosage may be delivered three times a day, two times a day, once a day, every other day, every third day, every week, every two weeks, every three weeks, or every four weeks. In certain embodiments, the desired dosage may be delivered using multiple administrations (e.g., two, three, four, five, six, seven, eight, nine, ten, eleven, twelve, thirteen, fourteen, or more administrations).

In an embodiment, the ratio by weight of the compound of Formula (A) or (B) of the present invention to any embodiment of the antineoplastic agent in a combination of the present invention is about 500:1 to about 1:500 such as about 500:1, about 400:1, about 300:1, about 200:1, about 100:1, about 90:1, about 80:1, about 70:1, about 60:1, about 50:1, about 40:1, about 30:1, about 20:1, about 10:1, about 9:1, about 8:1, about 7:1, about 6:1, about 5:1, about 4:1, about 3:1, about 2:1, about 1.5:1, about 1:1, about 1:0.9, about1: 0.8, about 1:0.7, about 1:0.6, about 1:0.5, about 1:0.4, about 1:0.3, about 1:0.2 or about 1:0.1, about 1:1.5, about 1:2, about 1:3, about 1:4, about 1:5, about 1:6, about 1:7, about 1:8, about 1:9, about 1:10, about 1:20, about 1:30, about 1:40, about 1:50, about 1:60, about 1:70, about 1:80, about 1:90, about 1:100, about 1:200, about 1:300, about 1:400 or about 1:500 including any numbers and ranges falling within these values.

### EXAMPLES

In order that the invention described herein may be more fully understood, the following examples are set forth. It should be understood that these examples are for illustrative purposes only and are not to be construed as limiting this invention in any manner.

### Example 1. Inhibition of Primary Patient Samples

Table 1 highlights a series of primary patient leukemia samples-both acute and chronic and treatment naïve and refractory and the effect of granaticin A, granaticin B, derivative 1, and derivative 2 to inhibit cellular proliferation. The IC50 data reveals that granaticin B is approximately one log more active than granaticin A against all the samples tested and inhibits cellular proliferation in the low nanomolar range. The two starred samples represent serial samples taken from the same patients after they received 2-3 cycles of additional high dose salvage chemotherapy and indicate that although refractory to salvage chemotherapy, the Cdc7 pathway remains a potentially efficacious target as the sensitivity to these compounds remains unchanged. Importantly, each patient sample was resistant to the prior therapy received using this assay.

TABLE 1
Inhibition of patient leukemia samples

| | IC₅₀ (microM) | | | |
|---|---|---|---|---|
| Leukemia sample | Granaticin A | Deriv. 1 | Deriv. 2 | Granaticin B |
| AML, de novo | 0.72 | 0.62 | 2.01 | 0.18 |
| AML, primary | 0.75 | 0.62 | 1.55 | 0.11 |
| refractory 1 | | | | |
| AML, primary | 0.63 | 0.47 | 1.44 | 0.10 |
| refractory 1* | | | | |
| AML, primary | 0.77 | 0.84 | 1.45 | 0.14 |
| refractory | | | | |
| AML, relapsed | 1.20 | 0.70 | 2.23 | 0.10 |
| AML, relapsed | 0.21 | 0.23 | 0.35 | 0.05 |
| AML, relapsed s/p | 1.88 | 1.86 | 8.10 | 0.62 |
| HSCT | | | | |
| AMMoL | 0.99 | 0.79 | 5.88 | 0.49 |
| ALL, Ph+ | 0.56 | 0.47 | 2.57 | 0.19 |
| Biphenotypic acute | 0.30 | 0.69 | 0.90 | 0.04 |
| leukemia 1 | | | | |
| Biphenotypic acute | 0.54 | 0.76 | 1.02 | 0.10 |
| leukemia 1* | | | | |
| CML, untreated | 0.26 | 0.33 | 1.24 | 0.08 |
| CLL, untreated, | 0.46 | 0.36 | 1.21 | 0.31 |
| chromosome 17p del. | | | | |
| PhALL3.1 | 0.14 | 0.12 | 0.47 | 0.03 |

*Indicates a second sample from the same patient following high-dose chemotherapy

TABLE 2

| | | IC₅₀ (microM) | |
|---|---|---|---|
| Cell Line | Description | Granaticin A | Granaticin B |
| ALL3 | Hematopoietic-Human | 0.14 | 0.03 |
| | Acute Lymphoblastic | | |
| | leukemia; Philadelphia | | |
| | chromosome positive | | |
| HL60 | Hematopoietic-Human | 0.14 | 0.09 |
| | Acute Promyelocytic | | |
| | leukemia | | |
| HL60 MX1 | Hematopoietic-Human | 0.17 | 0.08 |
| | Acute Promyelocytic | | |
| | leukemia; MDR variant of | | |
| | HL60 selected with | | |
| | mitoxantrone-clone 1 | | |
| HL60 MX2 | Hematopoietic-Human | 0.11 | 0.07 |
| | Acute Promyelocytic | | |
| | leukemia; MDR variant of | | |
| | HL60 selected with | | |
| | mitoxantrone-clone 2 | | |
| HL60 RV | Hematopoietic-Human | 0.09 | 0.07 |
| | Acute Promyelocytic | | |
| | leukemia: MDR variant of | | |
| | HL60 selected with | | |
| | vincristine | | |
| JEKO | Hematopoietic-Human | 0.04 | 0.02 |
| | Mantle Cell Lymphoma | | |
| JURKAT E61 | Hematopoietic-Human | 0.05 | 0.02 |
| | Acute Lymphoblastic T- | | |
| | cell leukemia | | |
| K562 | Hematopoietic-Human | 0.12 | 0.08 |
| | Chronic Myelogenous | | |
| | leukemia transformed to | | |
| | acute erythroleukemia | | |
| KASUMI4 | Hematopoietic-Human | 0.30 | 0.18 |
| | Chronic Myelogenous | | |
| | leukemia transformed to | | |
| | acute myeloid leukemia- | | |
| | overexpression of EVI1 | | |
| MEG01 | Hematopoietic-Human | 0.17 | 0.08 |
| | Chronic Myelogenous | | |
| | leukemia transformed to | | |
| | acute Megakaryoblastic | | |
| | leukemia | | |
| MOLT3 | Hematopoietic-Human | 0.03 | 0.01 |
| | Acute Lympholastic T-cell | | |
| | Leukemia | | |
| NCEB1 | Hematopoietic-Human | 0.33 | 0.26 |
| | Mantle Cell Lymphoma | | |
| 132 | Primary-Patient sample of | 0.13 | 0.01 |
| | Chronic Lymphocytic | | |
| | Leukemia | | |
| ALBU | Primary-Patient sample of | 0.43 | 0.03 |
| | essential thrombocythemia | | |
| | transformed to refractory | | |
| | acute myelogenous | | |
| | leukemia | | |
| DOGO | Primary-Patient sample of | 0.15 | 0.07 |
| | Chronic Lymphocytic | | |
| | Leukemia with trisomy of | | |
| | chromosome 12 and | | |
| | unmutated Ig heavy chain | | |
| | locus | | |
| GLHU | Primary-Patient sample of | 0.26 | 0.08 |
| | Chronic Myelogenous | | |
| | leukemia | | |
| JABR | Primary-Patient sample of | 0.21 | 0.05 |
| | relpased/refractory acute | | |
| | myelogenous leukemia | | |
| | with normal cytogenetics | | |
| | and Flt-3 ITD | | |
| JAKL | Primary-Patient sample of | 0.05 | 0.01 |
| | Chronic Lymphocytic | | |
| | Leukemia with trisomy of | | |
| | chromosome 12 and | | |
| | unmutated Ig heavy chain | | |
| | locus | | |
| JAMC | Primary-Patient sample of | 0.06 | 0.04 |
| | acute myelogenous | | |
| | leukemia with inversion of | | |
| | chromosome 16 | | |
| JAQU | Primary-Patient sample of | 0.05 | 0.01 |
| | relapsed/refractory pre-B | | |
| | cell acute lymphoblastic | | |
| | leukemia | | |
| JOBL | Primary-Patient sample of | 0.56 | 0.19 |
| | relapsed/refractory acute | | |
| | myelogenous leukemia | | |
| JOHO | Primary-Patient sample of | 0.88 | 0.17 |
| | primary refractory acute | | |
| | myelogenous leukemia | | |
| | with inv3 and monosomy 7 | | |
| | (refractory to allogeneic | | |
| | BMT) | | |
| JOQU | Primary-Patient sample of | 0.77 | 0.14 |
| | relapsed/refractory acute | | |
| | myelogenous leukemia | | |
| | with trisomy of | | |
| | chromosome #8 and Flt-3 | | |
| | ITD | | |
| JUCO | Primary-Patient sample of | 0.08 | 0.01 |
| | pre-B cell acute | | |
| | lymphoblastic leukemia | | |
| | with t(4; 11) involving the | | |
| | MLL gene | | |
| MAWI | Primary-Patient sample of | 0.72 | 0.18 |
| | relpased/refractory acute | | |
| | myelogenous leukemia | | |
| | with normal cytogenetics | | |
| MAWI B | Primary-Patient sample of | 0.42 | 0.16 |
| | secondary chronic | | |
| | myelogenous leukemia | | |
| MIHA | Primary-Patient sample of | 0.30 | 0.04 |
| | relapsed/refractory acute | | |
| | biphenotypic leukemia | | |
| | (patient was refractory to | | |
| | allogeneic bone marrow | | |
| | transplant) | | |
| PAPR | Primary-Patient sample of | 0.99 | 0.49 |
| | relapsed acute | | |
| | myelomonocytic leukemia | | |
| RADO | Primary-Patient sample of | 0.06 | 0.03 |
| | primary refractory acute | | |
| | myelogenous leukemia | | |
| | with multiple | | |
| | chromosomal | | |
| | abnormalities (−5, −7) | | |
| RORI | Primary-Patient sample of | 1.20 | 0.10 |
| | relapsed/refractory acute | | |
| | myelogenous leukemia | | |
| | with trisomy of | | |
| | chromosome #8 | | |
| SOPA | Primary-Patient sample of | 0.10 | 0.09 |
| | secondary acute | | |
| | myelogenous leukemia | | |
| | with chromsome 11q23 | | |
| | abnormality | | |
| STGL | Primary-Patient sample of | 1.88 | 0.62 |
| | refractory acute | | |
| | myelogenous leukemia | | |
| | with multiple | | |
| | chromosomal | | |
| | abnormalities(refractory to | | |
| | allogeneic BMT) | | |
| A2780 | Solid-Human ovarian | 0.12 | 0.02 |
| | carcinoma | | |
| A549 | Solid-Human lung | 0.28 | 0.09 |
| | adenocarcinoma; G12S | | |
| | KRAS mutation | | |
| BE(2)C | Solid-human | 0.07 | 0.04 |
| | neuroblastoma; p53 mutant | | |
| CWR22 | Solid-human prostate | 0.09 | 0.04 |
| | carcinoma | | |
| FUUR1 | Solid-human renal cell | 0.48 | 0.34 |
| | carcinoma with the | | |
| | reciprocal ASPL-TFE3 | | |
| | fusion transcript | | |
| H11-18 | Solid-Human lung | 0.42 | 0.06 |
| | adenocarcinoma; L858R | | |
| | EGFR mutation | | |
| H1650 | Solid- Human lung | 0.29 | 0.06 |
| | adenocarcinoma; DelE746- | | |
| | A750 EGFR mutation; | | |
| | wild-type RAS; PTEN null | | |
| H1734 | Solid-Human lung | 0.24 | 0.04 |
| | adenocarcinoma; G13C | | |
| | KRAS mutation | | |
| H1975 | Solid-Human lung | 0.12 | 0.03 |
| | adenocarcinoma; | | |
| | T790M/L858R EGFR | | |
| | mutation; R273H p53 | | |
| | mutation; PTEN null; | | |
| | wild-type KRAS | | |
| H2030 | Solid-human lung | 0.49 | 0.22 |
| | adenocarcinoma; wild-type | | |
| | EGFR; G12C KRAS | | |
| | mutation; G262V P53 | | |
| | mutation; PTEN null | | |
| H2122 | Solid-human lung | 1.19 | 0.21 |
| | adenocarcinoma; G12C | | |
| | KRAS mutation; Q16L | | |
| | and C176F P53 mutations | | |
| H23 | Solid-Human lung | 0.12 | 0.05 |
| | adenocarcinoma; G12c | | |
| | KRAS mutation; M246I | | |
| | P53 mutation | | |
| H2444 | Solid-human lung | 0.31 | 0.03 |
| | adenocarcinoma; G12V | | |
| | KRAS mutation | | |
| H3255 | Solid-Human lung | 0.21 | 0.07 |
| | adenocarcinoma; L858R | | |
| | EGFR mutation; wild-type | | |
| | KRAS; wild-type P53 | | |
| H358 | Solid-Human lung | 0.21 | 0.05 |
| | adenocarcinoma; G13C | | |
| | KRAS mutation | | |
| H460 | Solid-human lung | 0.39 | 0.08 |
| | adenocarcinoma; Q61H | | |
| | KRAS mutation | | |
| H820 | Solid-human lung | 0.09 | 0.05 |
| | adenocarcinoma; DelE746- | | |
| | E749 and T790M | | |
| | EGFR mutations | | |
| HCC4011 | Solid-Human lung | 0.14 | 0.05 |
| | adenocarcinoma; L858R | | |
| | EGFR mutation; wild-type | | |
| | KRAS | | |
| HCC827 | Solid-human lung | 0.28 | 0.12 |
| | adenocarcinoma; DelE746- | | |
| | A750 EGFR mutation | | |
| HELA N10 | Solid-human cervical | 1.27 | 0.97 |
| | adenocarcinoma (positive | | |
| | for human papillomavirus | | |
| | infection) | | |
| HPLD1 | Solid-Immortalized human | 0.18 | 0.04 |
| | bronchiolar epithelial cell | | |
| | with SV-40 Large T | | |
| | antigen | | |
| HTB15 | Solid-Human | 0.65 | 0.46 |
| | Glioblastoma (U-118MG), | | |
| | classified as Grade IV | | |
| JNDSRCT1 | Solid-Human desmoplastic | 0.07 | 0.05 |
| | small round cell tumor cell | | |
| | line | | |
| MCF-10A | Solid-human normal | 0.26 | 0.24 |
| | mammary epithelium | | |
| MCF-7 | Solid-human invasive | 0.17 | 0.13 |
| | breast ductal carcinoma, | | |
| | estrogen and progesterone | | |
| | receptor positive | | |
| MDA-MB-231 | Solid-human metastatic | 0.47 | 0.29 |
| | breast adenocarcinoma; | | |
| | estrogen, progesterone, | | |
| | and HER2/NEU receptor | | |
| | negative | | |
| MESO47 | Solid-human | 0.61 | 0.38 |
| | mesothelioma; WT-1 | | |
| | overexpression | | |
| OVCAR3 | Solid-human ovarian | 0.21 | 0.11 |
| | adenocarcinoma | | |
| PC9 | Solid-Human lung | 0.30 | 0.07 |
| | adenocarcinoma; DelE746- | | |
| | A750 EGFR mutation | | |
| RB355 | Solid-Human | 0.09 | 0.04 |
| | retinoblastoma | | |
| RH30 | Solid-Human | 0.13 | 0.08 |
| | rhabdomyosarcoma; P53 | | |
| | mutation | | |
| SKMEL28 | Solid-Human melanoma | 0.54 | 0.34 |
| SKOV3 | Solid-Human | 0.12 | 0.04 |
| | adenocarcinoma from the | | |
| | ovary derived from ascites; | | |
| | hypodiploid | | |
| SW1736 | Solid-Human anaplastic | 0.16 | 0.02 |
| | thyroid carcinoma with | | |
| | BRAF V600E mutation | | |
| TC71 | Solid-Human Ewing's | 0.15 | 0.09 |
| | sarcoma | | |
| Y79 | Solid-Human | 0.06 | 0.05 |
| | retinoblastoma | | |

### Example 3. Stability Studies Using Granaticin A

Standard liver microsome stability tests were performed with mouse and human microsomes with granaticin A and revealed that this drug is cleared quickly by the liver. The stability of granaticin A was determined in mouse and human liver microsomes. In mouse liver microsomes, recovery at 30 minutes was 3.68%. In mouse liver microsomes, recovery at 60 minutes was 3.84%. In human liver microsomes, recovery at 30 minutes was 23.89%. In human liver microsomes, recovery at 60 minutes was 12.01%.

### Example 4. Preliminary Screening of Granaticin B

Standard solubility studies were conducted with granaticin B. In aqueous solution, 4.0 mg/ml of granaticin B is soluble. In alcohol, >188 mg/ml of granaticin B is soluble. In methanol, >252 mg/ml of granaticin B is soluble.

Different formulations of granaticin B were also tested and PK data measured (see Table 3).

TABLE 3
Preliminary Mouse PK Screening

| Granaticin B, | | | | |
|---|---|---|---|---|
| Mouse PK | AUC_{inf} | Half-life | Cₘₐₓ | CL |
| screening 5 mg/kg | (hr − μg/mL) | (t_{2/1}^{ß}) | (μg/mL) | (mL/hr/kg) |
| A: 1.25% of Ethanol & | 3532.5 | 6.9 | 2834.7 | 1415.4 |
| Tween-20 | | | | |
| B: 0.6% GDO-12 | 477981.5 | 3445.5 | 2537.2 | 10.5 |
| C: 5% GDP-12 | 2689.9 | 6.6 | 2449.9 | 1858.8 |
| D: DMSO | 2724.5 | 2.1 | 2210.9 | 1835.2 |

### Example 5 In Vitro Inhibition of Kasumi-1 and CESS cells

### Methods and Materials

### Cell Culture

Kasumi-1 and CESS cells were cultured in RPMI medium containing 30% and 10% fetal bovine serum (FBS), respectively. For each experimental condition, cells were seeded in 96-well plate at a density of 1×10\hat{ }5 cells/ml for each well.

### Drug Treatments

The three drugs, LBS-007 (007) is granaticin B, Venetoclax (Ven), and Azacitidine (Aza), were administered at fixed concentrations to the cells.

### Control Group: Cells Received no Drug Treatment

Single treatment Group: Cells were treated with single drug. (007, Ven or Aza)
Double Treatment Group: Cells were treated with a combination of two drugs. (007+Ven, 007+Aza, Ven+Aza)
Triple Treatment Group: Cells were simultaneously treated with all three drugs. (007+Ven+Aza)

### Dose and Duration

Single treatment and combinations of LBS-007 (007), Venetoclax (Ven) and Azacytidine (Aza) at 40 nM, 20 nM and 500 nM respectively were treated for a duration of 72 hours.

### Cell Viability Assessment

Cell viability was assessed using the CCK-8 assay. Following the completion of the 72-hour drug treatments, CCK-8 reagent was added to each well at 10% of the total volume, and the cells were incubated for an additional 1.5 hours. The absorbance of 450 nm was measured using a SpectraMax iD3 microplate reader (Molecular Devices).

### Statistical Analysis

Statistical analyses were conducted to assess the significance of observed differences among the experimental groups. Initially, a one-way ANOVA was employed to determine whether there were overall differences among the control, double treatment, and triple treatment groups. Subsequently, Student's two-tailed t-test was used to analyze data sets. Results with * p<0.05, **p<0.01, and ***p<0.001 were considered significant when compared to Control (Ctrl). Results with #p<0.05, ##p<0.01, and ###p<0.001 were considered significant when compared as indicated. All experimental data are reported as the mean and the error bars represent the experimental standard error (±standard deviation, SD).

The experiment focused on two types of acute myeloid leukemia (AML) cell lines, Kasumi-1 and CESS, representing different subtypes within the AML spectrum. The Kasumi-1 cells are myeloblast cells that was isolated from the peripheral blood of an acute myeloblastic leukemia Asian male patient. CESS cells are lymphoblast cells isolated from the blood of a White male with myelomonocytic leukemia. The results are shown in FIGS. 25 and 26.

As shown in FIG. 25 for cell viability experiment on Kasumi-1 cells, Aza alone had almost no effect, and LBS-007 and Ven each alone provided about the same effectiveness on Kasumi-1 cells with cell viability at about 60%. Notably, combination of Aza+Ven as well as 007+Aza provided no additional benefit as compared to LBS-007 or Ven alone, illustrating that double combination therapy do not always result in better results. However, when LBS-007 is combined with Ven, cell viability improved almost doubled, showing that combination of LBS-007 and Ven provides synergistic effect. Surprisingly, although Aza alone is shown to have no effect on cell viability, the addition of Aza to LBS-007 and Ven provided nearly 20% improvement to cell viability results.

As shown in FIG. 26 for cell viability experiment on CESS cells, Ven and Aza alone showed little or no effect on cell viability at the concentration tested whereas LBS-007 demonstrated substantial effect on cell viability at about 50%. Although showing no effect individually, Ven+Aza in combination achieved about 50% cell viability. However, the combinations LBS-007+Aza and LBS-007+Ven doubled effect on cell viability compared to that of the Ven+Aza combination to about 25% cell viability. Most notably, treatment of the CESS cells with the triple combination of LBS-007+Aza+Ven resulted in cell viability of about 7%.

The results of our investigation demonstrated that the triple treatment approach, combining LBS-007, Venetoclax (Ven), and Azacitidine (Aza), proved to be the most effective in inducing cytotoxicity in these AML cell lines. This finding underscores the potential of the triple treatment strategy as a promising combination therapeutic avenue for AML.

What is claimed is:

1. A method of treatment of a proliferative disorder of a subject comprising step of administration of a pharmaceutical composition comprising a therapeutically effective amount of a combination of (a) a compound of Formula (A) or (B) or a pharmaceutically acceptable salt thereof and (b) one or more antineoplastic agents to the subject wherein:
A compound of Formula (A) or (B): 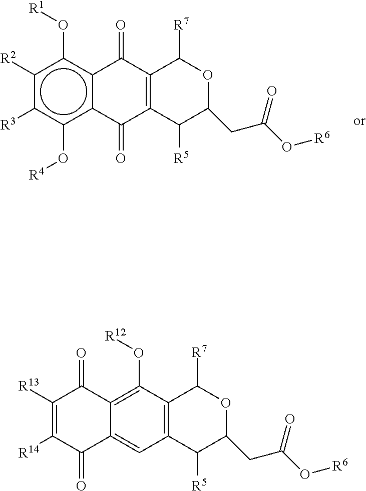
each instance of R¹ and R⁴ is independently selected from the group consisting of hydrogen, carbonyl, silyl, optionally substituted alkyl, optionally substituted alkenyl, optionally substituted alkynyl, optionally substituted carbocyclyl, optionally substituted heterocyclyl, optionally substituted aryl, optionally substituted heteroaryl;
each instance of R² and R³ is independently selected from the group consisting of hydrogen, halogen, —OH, substituted hydroxyl, —SH, substituted thiol, —NH₂, substituted amino, —CN, —NO₂, carbonyl, silyl, sulfinyl, sulfonyl, optionally substituted alkyl, optionally substituted alkenyl, optionally substituted alkynyl, optionally substituted carbocyclyl, optionally substituted heterocyclyl, optionally substituted aryl, and optionally substituted heteroaryl; or R² and R³ are joined to form an optionally substituted carbocyclyl, optionally substituted heterocyclyl, optionally substituted aryl, or optionally substituted heteroaryl group;
R⁵ is hydrogen and R⁶ is selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl; or R⁵ and R⁶ are joined to form a direct bond;
R⁷ is hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl;
R¹² is hydrogen, carbonyl, silyl, optionally substituted alkyl, optionally substituted alkenyl, optionally substituted alkynyl, optionally substituted carbocyclyl, optionally substituted heterocyclyl, optionally substituted aryl, optionally substituted heteroaryl; and
each instance of R¹³ and R¹⁴ is independently selected from the group consisting of hydrogen, halogen, —OH, substituted hydroxyl, —SH, substituted thiol, —NH₂, substituted amino, —CN, —NO₂, carbonyl, silyl, sulfinyl, sulfonyl, optionally substituted alkyl, optionally substituted alkenyl, optionally substituted alkynyl, optionally substituted carbocyclyl, optionally substituted heterocyclyl, optionally substituted aryl, and optionally substituted heteroaryl; or R¹³ and R¹⁴ are joined to form an optionally substituted carbocyclyl, optionally substituted heterocyclyl, optionally substituted aryl, or optionally substituted heteroaryl group.

2. The method of claim 1, wherein the compound of formula A or B comprises granaticin B wherein the granticin B comprises amorphous form of the granticin B or any crystalline form of the granaticin B.

3. The method of claim 2, wherein the compound of formula A or B comprises crystalline form A of granaticin B.

4. The method of claim 1, wherein the one or more antineoplastic agents comprise hydroxyurea, granulocyte colony stimulating factor (G-CSF), BCL2 inhibitor, hypomethylating agent, chemotherapeutic agent or a combination thereof.

5. The method of claim 4, wherein the BCL2 inhibitor comprises venetoclax

6. The method of claim 4, wherein the hypomethylating agent comprises azacitidine, decitabine or a combination thereof.

7. The method of claim 4, wherein the chemotherapeutic agent comprises cytarabine, daunorbicin, idarubicin, anthracycline, cladribine or a combination thereof.

8. The method of claim 1, wherein the compound of Formula (A) or (B) comprises granticin B and the one or more antineoplastic agents comprises azacitidine and venetoclax.

9. The method of claim 1, wherein the one or more antineoplastic agent comprises fludarabine, arabinofuranosyl cytidine, granulocyte colony-stimulating factor and idarubicin (FLAG-IDA).

10. The method of claim 9, wherein administration step comprises administration of the standard FLAG-IDA chemotherapy regimen administered in combination with the compound of Formula (A) or (B).

11. The method of claim 9, wherein administration step comprises administration of the standard FLAG-IDA chemotherapy regimen administered in combination with the compound of Formula (A) or (B) and venetoclax.

12. The method of claim 1, wherein the one or more antineoplastic agent comprises cytarabine and anthracycline antibiotic and wherein administration step comprises the standard 7 days of standard-dose cytarabine followed by 3 days of anthracycline antibiotic chemotherapy regimen administered in combination with the compound of Formula (A) or (B).

13. The method of claim 4, wherein the chemotherapeutic agent comprises anti-estrogens such as tamoxifen, raloxifene, and megestrol, LHRH agonists such as goscrclin and leuprolide, anti-androgens such as flutamide and bicalutamide, photodynamic therapies such as vertoporfin (BPD-MA), phthalocyanine, photosensitizer Pc4, and demethoxy-hypocrellin A (2BA-2-DMHA, nitrogen mustards such as cyclophosphamide, ifosfamide, trofosfamide, chlorambucil, estramustine, and melphalan, nitrosoureas such as carmustine (BCNU) and lomustine (CCNU), alkylsulphonates such as busulfan and treosulfan, triazenes such as dacarbazine, temozolomide, platinum containing compounds such as cisplatin, carboplatin, oxaliplatin, vinca alkaloids such as vincristine, vinblastine, vindesine, and vinorelbine, taxoids such as paclitaxel or a paclitaxel equivalent such as nanoparticle albumin-bound paclitaxel (ABRAXANE), docosahexaenoic acid bound-paclitaxel (DHA-paclitaxel), Taxoprexin, polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, 2′-paclitaxel methyl 2-glucopyranosyl succinate; docetaxel, taxol, epipodophyllins such as etoposide, etoposide phosphate, teniposide, topotecan, 9-aminocamptothecin, camptoirinotecan, irinotecan, crisnatol, mytomycin C, anti-metabolites, DHFR inhibitors such as methotrexate, dichloromethotrexate, trimetrexate, edatrexate, IMP dehydrogenase inhibitors such as mycophenolic acid, tiazofurin, ribavirin, and EICAR, ribonuclotide reductase inhibitors such as hydroxyurea and deferoxamine, uracil analogs such as 5-fluorouracil (5-FU), floxuridine, doxifluridine, ratitrexed, tegafur-uracil, capecitabine, cytosine analogs such as cytarabine (ara C), cytosine arabinoside, and fludarabine, purine analogs such as mercaptopurine and Thioguanine, Vitamin D3 analogs such as EB 1089, CB 1093, and KH 1060, isoprenylation inhibitors such as lovastatin, dopaminergic neurotoxins such as 1-methyl-4-phenylpyridinium ion, cell cycle inhibitors such as staurosporine, actinomycin such as actinomycin D, dactinomycin, bleomycin such as bleomycin A2, bleomycin B2, peplomycin, anthracycline such as daunorubicin, doxorubicin, pegylated liposomal doxorubicin, idarubicin, epirubicin, pirarubicin, zorubicin, mitoxantrone, MDR inhibitors such as verapamil, Ca²⁺ ATPase inhibitors such as thapsigargin, imatinib, thalidomide, lenalidomide, tyrosine kinase inhibitors such as axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228, proteasome inhibitors such as bortezomib (VELCADE), mTOR inhibitors such as rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, Gemtuzumab, ozogamicin, Idarubicin, CPX-351, Enasidenib (IDH2 inibitor), FTL3 inhibitors such as Sunitinib, Midostaurin, Lestaurtinib, Crenolanib, Gilteritinib, Sorafenib, Ponatinib, Quizartinib) Ivosidenib (IDH1 inhibitor), Venetoclax (BCL-2 inhibitor) and Glasdegib, rituximab, ofatuzumab (Anti-CD20), inotuzumab ozogamicin (Anti-CD22), blinatumomab (Anti-CD19), and Tisagenlecleucel, tyrosine kinase inhibitor (Imatinib, Dasatinib, Nilotinib, Ponatinib, Bosatinib, Asciminib) with/without chemotherapy (Clofarabine, Nelarabine, Liposomal vincristine, MOpAD, Hyper-CVAD, BFM, etc) or steroid, inotuzumab ozogamicin, blinatumomab, Tisagenlecleucel, or a combination thereof.

14. The method of claim 1, wherein the proliferative disorder comprises cancer.

15. The method of claim 1, wherein the proliferation disorder comprises blood cancers wherein the blood cancers comprise acute myeloid leukemia (AML), acute lymphoblastic leukemia (ALL), chronic myeloid leukemia (CML) and/or chronic lymphoblastic leukemia (CLL).

16. The method of claim 14, wherein the cancer comprises a mutation.

17. The method of claim 16, wherein the genetic mutation comprises a RAS mutation, an EGFR mutation, a KRAS mutation, a p53 mutation, a BRAF mutation, a EVI1 mutation, a Flt-3 mutation, WT-1 mutation, a cyclin D mutation, a PTEN mutation, an ALB kinase mutation, a chromosomal abnormality or a combination thereof.

18. The method of claim 14, wherein the cancer is a multi-drug resistant (MDR) cancer.

19. The method of claim 14, wherein the cancer is relapsed and/or refractory cancer.

20. The method of claim 1 further comprising the step of administration of radiation.

21. The method of claim 1 wherein the ratio by weight of the compound of Formula (A) or (B) to the antineoplastic agent is about 500:0.1 to about 0.1:500.

22. The method of claim 1, wherein the compound of Formula (A) or (B) is of the Formula (A-3): 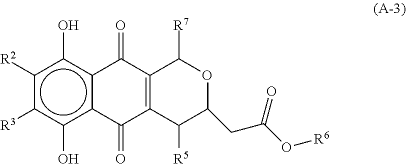
or a pharmaceutically acceptable salt thereof.

23. The method of claim 1, wherein the compound of Formula (A) or (B) is of the Formula (A-7): 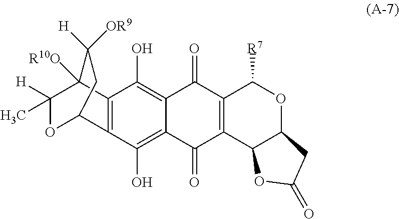
or a pharmaceutically acceptable salt thereof,
wherein each instance of R⁹ and R¹⁰ is independently selected from hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, carbonyl, silyl, sulfonyl, and sulfinyl.

24. The method of claim 1, wherein the compound of Formula (A) or (B) is of the Formula (B-1): 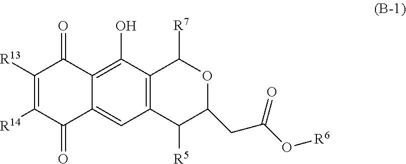
or a pharmaceutically acceptable salt thereof.

25. The method of claim 1, wherein the compound of Formula (A) or (B) is selected from the group consisting of: 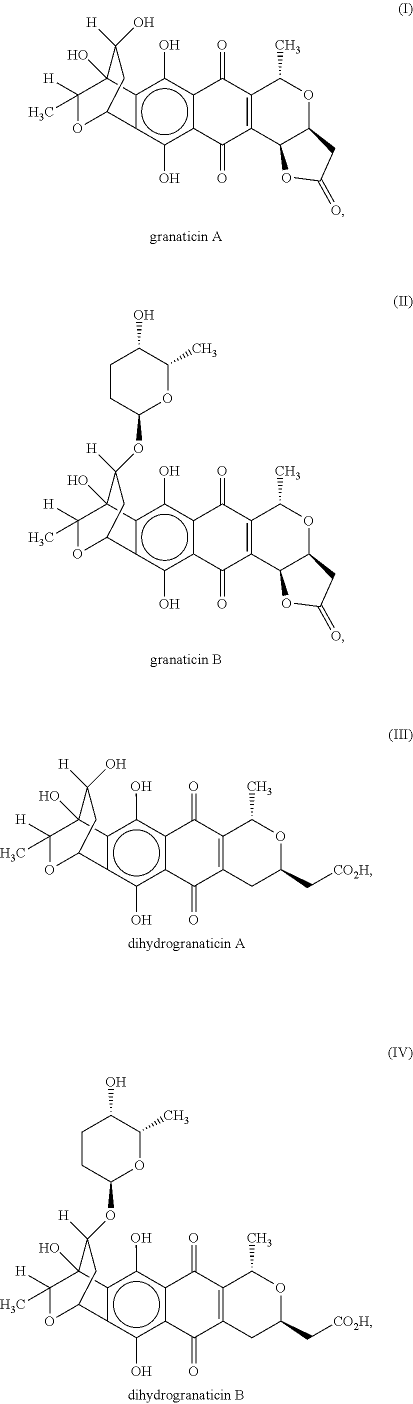 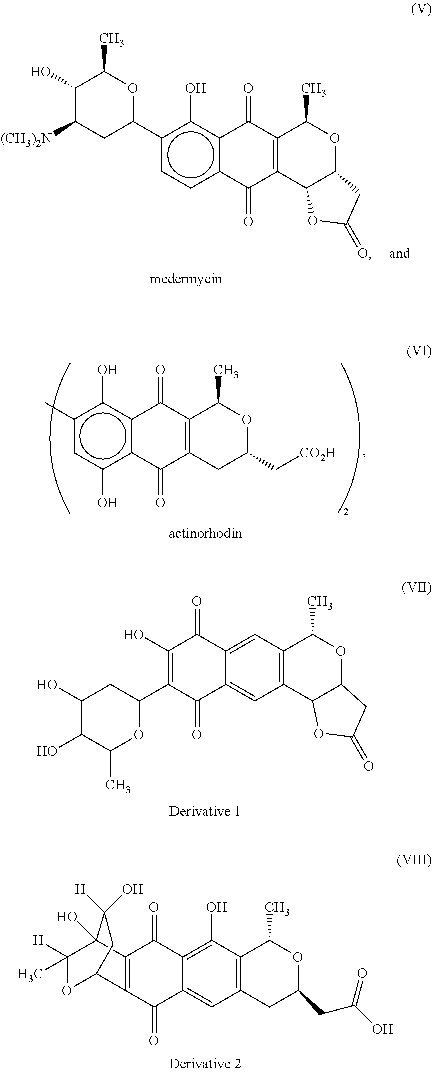
and pharmaceutically acceptable salts thereof.

26. The method of claim 1, wherein the therapeutically effective amount of the pharmaceutical composition is administered to the subject by intravenous injection, intraarterial injection, intraperitoneal injection, intrapleural injection, intracardiac injection, or intrapericardial injection.

27. The method of claim 1, wherein the compound of Formula (A) or (B) is administered to a subject simultaneously with the antineoplastic agent.

28. The method of claim 1, wherein the compound of Formula (A) or (B) is administered to a subject separately sequentially before the administration of antineoplastic agent with time interval in between the two administrations of about 0.5 hour, about 1 hour, about 2 hours, about 5 hours, about 10 hours, about 20 hours, about 24 hours, about 2 days, about 3 days, about 4 days, about 5 days, about 6 days, about 7 days, about 2 weeks, about 3 weeks or about 4 weeks, about 6 weeks or about 8 weeks.

29. The method of claim 1, wherein the compound of Formula (A) or (B) is administered to a subject after the administration of antineoplastic agent with time interval in between the two administrations of about 1 hour, about 2 hours, about 5 hours, about 10 hours, about 20 hours, about 24 hours, about 2 days, about 3 days, about 4 days, about 5 days, about 6 days, about 7 days, about 2 weeks, about 3 weeks, about 4 weeks, about 6 weeks or about 8 weeks.

30. A pharmaceutical composition comprising a combination of (a) a compound of Formula (A) or (B) and (b) one or more antineoplastic agents, wherein the compound of Formula (A) or (B) and the one or more antineoplastic agents are present in each case in free form, in the form of a pharmaceutically acceptable salt, or hydrate thereof, and wherein
A compound of Formula (A) or (B): 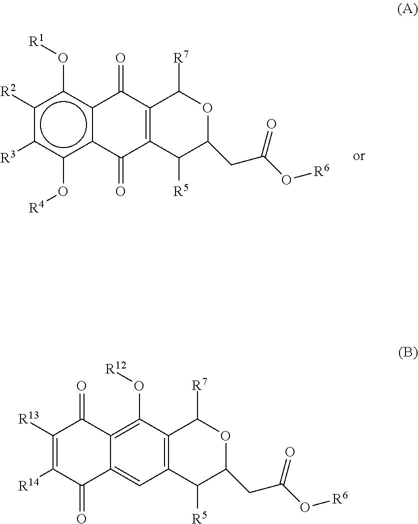
each instance of R¹ and R⁴ is independently selected from the group consisting of hydrogen, carbonyl, silyl, optionally substituted alkyl, optionally substituted alkenyl, optionally substituted alkynyl, optionally substituted carbocyclyl, optionally substituted heterocyclyl, optionally substituted aryl, optionally substituted heteroaryl;
each instance of R² and R³ is independently selected from the group consisting of hydrogen, halogen, —OH, substituted hydroxyl, —SH, substituted thiol, —NH₂, substituted amino, —CN, —NO₂, carbonyl, silyl, sulfinyl, sulfonyl, optionally substituted alkyl, optionally substituted alkenyl, optionally substituted alkynyl, optionally substituted carbocyclyl, optionally substituted heterocyclyl, optionally substituted aryl, and optionally substituted heteroaryl; or R² and R³ are joined to form an optionally substituted carbocyclyl, optionally substituted heterocyclyl, optionally substituted aryl, or optionally substituted heteroaryl group;
R⁵ is hydrogen and R⁶ is selected from the group consisting of hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl; or R⁵ and R⁶ are joined to form a direct bond;
R⁷ is hydrogen, optionally substituted alkyl, optionally substituted alkenyl, optionally substituted alkynyl, optionally substituted carbocyclyl, optionally substituted heterocyclyl, optionally substituted aryl, and optionally substituted heteroaryl;
R¹² is hydrogen, carbonyl, silyl, optionally substituted alkyl, optionally substituted alkenyl, optionally substituted alkynyl, optionally substituted carbocyclyl, optionally substituted heterocyclyl, optionally substituted aryl, optionally substituted heteroaryl; and
each instance of R¹³ and R¹⁴ is independently selected from the group consisting of hydrogen, halogen, —OH, substituted hydroxyl, —SH, substituted thiol, —NH₂, substituted amino, —CN, —NO₂, carbonyl, silyl, sulfinyl, sulfonyl, optionally substituted alkyl, optionally substituted alkenyl, optionally substituted alkynyl, optionally substituted carbocyclyl, optionally substituted heterocyclyl, optionally substituted aryl, and optionally substituted heteroaryl; or R¹³ and R¹⁴ are joined to form an optionally substituted carbocyclyl, optionally substituted heterocyclyl, optionally substituted aryl, or optionally substituted heteroaryl group.

31. The pharmaceutical composition of claim 30, wherein the compound of formula A or B comprises granaticin B wherein the ganaticin B comprises amourphous form of the granaticin B or any crystalline form of the granaticin B.

32. The pharmaceutical composition of claim 31, wherein the compound of formula A or B comprises crystalline form A of granaticin B.

33. The pharmaceutical composition of claim 30, wherein the one or more antineoplastic agents comprise hydroxyurea, granulocyte colony stimulating factor (G-CSF), BCL2 inhibitor, hypomethylating agent, chemotherapeutic agent or a combination thereof.

34. The pharmaceutical composition of claim 33, wherein the BCL2 inhibitor comprises venetoclax

35. The pharmaceutical composition of claim 33, wherein the hypomethylating agent comprises azacitidine, decitabine or a combination thereof.

36. The pharmaceutical composition of claim 33, wherein the chemotherapeutic agent comprises cytarabine, daunorbicin, idarubicin, anthracycline, cladribine or a combination thereof.

37. The pharmaceutical composition of claim 30, wherein the compound of Formula (A) or (B) comprises granticin B and the one or more antineoplastic agents comprises azacitidine and venetoclax.

38. The pharmaceutical composition of claim 30, wherein the one or more antineoplastic agent comprises fludarabine, arabinofuranosyl cytidine, granulocyte colony-stimulating factor and idarubicin (FLAG-IDA).

39. The pharmaceutical composition of claim 33, wherein the chemotherapeutic agent comprises anti-estrogens such as tamoxifen, raloxifene, and megestrol, LHRH agonists such as goscrclin and leuprolide, anti-androgens such as flutamide and bicalutamide, photodynamic therapies such as vertoporfin (BPD-MA), phthalocyanine, photosensitizer Pc4, and demethoxy-hypocrellin A (2BA-2-DMHA, nitrogen mustards such as cyclophosphamide, ifosfamide, trofosfamide, chlorambucil, estramustine, and melphalan, nitrosoureas such as carmustine (BCNU) and lomustine (CCNU), alkylsulphonates such as busulfan and treosulfan, triazenes such as dacarbazine, temozolomide, platinum containing compounds such as cisplatin, carboplatin, oxaliplatin, vinca alkaloids such as vincristine, vinblastine, vindesine, and vinorelbine, taxoids such as paclitaxel or a paclitaxel equivalent such as nanoparticle albumin-bound paclitaxel (ABRAXANE), docosahexaenoic acid bound-paclitaxel (DHA-paclitaxel), Taxoprexin, polyglutamate bound-paclitaxel (PG-paclitaxel, paclitaxel poliglumex, CT-2103, XYOTAX), the tumor-activated prodrug (TAP) ANG1005 (Angiopep-2 bound to three molecules of paclitaxel), paclitaxel-EC-1 (paclitaxel bound to the erbB2-recognizing peptide EC-1), and glucose-conjugated paclitaxel, 2′-paclitaxel methyl 2-glucopyranosyl succinate; docetaxel, taxol, epipodophyllins such as etoposide, etoposide phosphate, teniposide, topotecan, 9-aminocamptothecin, camptoirinotecan, irinotecan, crisnatol, mytomycin C, anti-metabolites, DHFR inhibitors such as methotrexate, dichloromethotrexate, trimetrexate, edatrexate, IMP dehydrogenase inhibitors such as mycophenolic acid, tiazofurin, ribavirin, and EICAR, ribonuclotide reductase inhibitors such as hydroxyurea and deferoxamine, uracil analogs such as 5-fluorouracil (5-FU), floxuridine, doxifluridine, ratitrexed, tegafur-uracil, capecitabine, cytosine analogs such as cytarabine (ara C), cytosine arabinoside, and fludarabine, purine analogs such as mercaptopurine and Thioguanine, Vitamin D3 analogs such as EB 1089, CB 1093, and KH 1060, isoprenylation inhibitors such as lovastatin, dopaminergic neurotoxins such as 1-methyl-4-phenylpyridinium ion, cell cycle inhibitors such as staurosporine, actinomycin such as actinomycin D, dactinomycin, bleomycin such as bleomycin A2, bleomycin B2, peplomycin, anthracycline such as daunorubicin, doxorubicin, pegylated liposomal doxorubicin, idarubicin, epirubicin, pirarubicin, zorubicin, mitoxantrone, MDR inhibitors such as verapamil, Ca²⁺ ATPase inhibitors such as thapsigargin, imatinib, thalidomide, lenalidomide, tyrosine kinase inhibitors such as axitinib (AG013736), bosutinib (SKI-606), cediranib (RECENTIN™, AZD2171), dasatinib (SPRYCEL®, BMS-354825), erlotinib (TARCEVA®), gefitinib (IRESSA®), imatinib (Gleevec®, CGP57148B, STI-571), lapatinib (TYKERB®, TYVERB®), lestaurtinib (CEP-701), neratinib (HKI-272), nilotinib (TASIGNA®), semaxanib (semaxinib, SU5416), sunitinib (SUTENT®, SU11248), toceranib (PALLADIA®), vandetanib (ZACTIMA®, ZD6474), vatalanib (PTK787, PTK/ZK), trastuzumab (HERCEPTIN®), bevacizumab (AVASTIN®), rituximab (RITUXAN®), cetuximab (ERBITUX®), panitumumab (VECTIBIX®), ranibizumab (Lucentis®), nilotinib (TASIGNA®), sorafenib (NEXAVAR®), everolimus (AFINITOR®), alemtuzumab (CAMPATH®), gemtuzumab ozogamicin (MYLOTARG®), temsirolimus (TORISEL®), ENMD-2076, PCI-32765, AC220, dovitinib lactate (TKI258, CHIR-258), BIBW 2992 (TOVOK™), SGX523, PF-04217903, PF-02341066, PF-299804, BMS-777607, ABT-869, MP470, BIBF 1120 (VARGATEF®), AP24534, JNJ-26483327, MGCD265, DCC-2036, BMS-690154, CEP-11981, tivozanib (AV-951), OSI-930, MM-121, XL-184, XL-647, and/or XL228, proteasome inhibitors such as bortezomib (VELCADE), mTOR inhibitors such as rapamycin, temsirolimus (CCI-779), everolimus (RAD-001), ridaforolimus, AP23573 (Ariad), AZD8055 (AstraZeneca), BEZ235 (Novartis), BGT226 (Norvartis), XL765 (Sanofi Aventis), PF-4691502 (Pfizer), GDC0980 (Genetech), SF1126 (Semafoe) and OSI-027 (OSI), oblimersen, gemcitabine, carminomycin, leucovorin, pemetrexed, cyclophosphamide, dacarbazine, procarbizine, prednisolone, dexamethasone, campathecin, plicamycin, asparaginase, aminopterin, methopterin, porfiromycin, melphalan, leurosidine, leurosine, chlorambucil, trabectedin, procarbazine, discodermolide, carminomycin, aminopterin, and hexamethyl melamine, Gemtuzumab, ozogamicin, Idarubicin, CPX-351, Enasidenib (IDH2 inibitor), FTL3 inhibitors such as Sunitinib, Midostaurin, Lestaurtinib, Crenolanib, Gilteritinib, Sorafenib, Ponatinib, Quizartinib) Ivosidenib (IDH1 inhibitor), Venetoclax (BCL-2 inhibitor) and Glasdegib, rituximab, ofatuzumab (Anti-CD20), inotuzumab ozogamicin (Anti-CD22), blinatumomab (Anti-CD19), and Tisagenlecleucel, tyrosine kinase inhibitor (Imatinib, Dasatinib, Nilotinib, Ponatinib, Bosatinib, Asciminib) with/without chemotherapy (Clofarabine, Nelarabine, Liposomal vincristine, MOpAD, Hyper-CVAD, BFM, etc) or steroid, inotuzumab ozogamicin, blinatumomab, Tisagenlecleucel, or a combination thereof.

40. The pharmaceutical composition of claim 30 wherein the ratio by weight of the compound of Formula (A) or (B) to the antineoplastic agent is about 500:0.1 to about 0.1:500.

41. The pharmaceutical composition of claim 30, wherein the compound of Formula (A) or (B) is of the Formula (A-3): 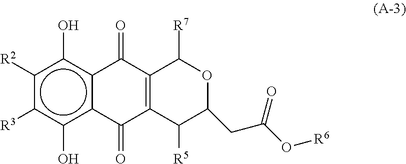
or a pharmaceutically acceptable salt thereof.

42. The pharmaceutical composition of claim 30, wherein the compound of Formula (A) or (B) is of the Formula (A-7): 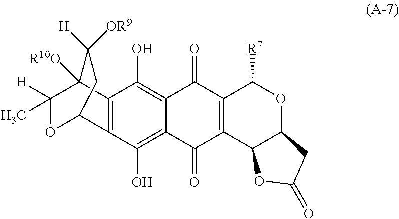
or a pharmaceutically acceptable salt thereof,
wherein each instance of R⁹ and R¹⁰ is independently selected from hydrogen, optionally substituted alkyl, optionally substituted alkenyl, optionally substituted alkynyl, optionally substituted carbocyclyl, optionally substituted heterocyclyl, optionally substituted aryl, optionally substituted heteroaryl, carbonyl, silyl, sulfonyl, and sulfinyl.

43. The pharmaceutical composition of claim 30, wherein the compound of Formula (A) or (B) is of the Formula (B-1): 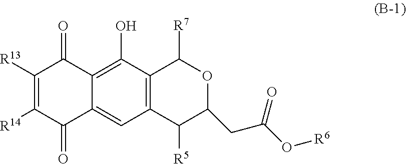
or a pharmaceutically acceptable salt thereof.

44. The pharmaceutical composition of claim 30, wherein the compound of Formula (A) or (B) is selected from the group consisting of: 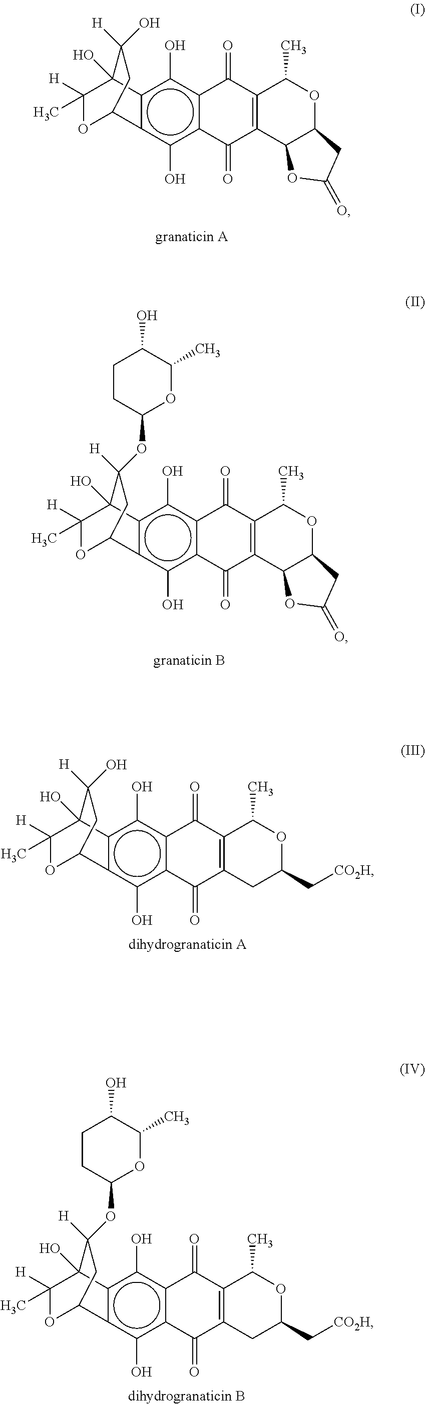 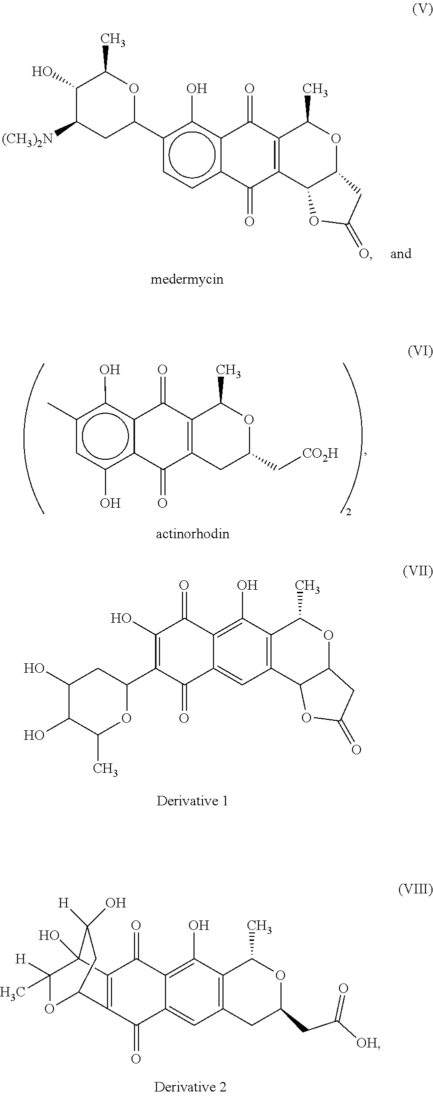
and pharmaceutically acceptable salts thereof.
